(19) United States
(12) Patent Application Publication
(10) Pub. No.: US 2013/0267513 A1
(43) Pub. Date: Oct. 10, 2013

(54) PYRAZOLOPYRIDINES AS INHIBITORS OF THE KINASE LRRK2

(71) Applicants: Brayn Chan, San Francisco, CA (US); Anthony Estrada, San Francisco, CA (US); Zachary Sweeney, San Francisco, CA (US); Edward Giles Mciver, London (GB); Stephen Lewis, San Francisco, CA (US)

(72) Inventors: Brayn Chan, San Francisco, CA (US); Anthony Estrada, San Francisco, CA (US); Zachary Sweeney, San Francisco, CA (US); Edward Giles Mciver, London (GB); Stephen Lewis, San Francisco, CA (US)

(73) Assignee: MEDICAL RESEARCH COUNCIL TECHNOLOGY, London (GB)

(21) Appl. No.: 13/697,878

(22) PCT Filed: May 16, 2011
(86) PCT No.: PCT/GB11/50937

§ 371 Date: Jun. 20, 2013

(30) Foreign Application Priority Data
May 14, 2010 (GB) 1008134.7

(51) Int. Cl. A61K 31/437; A61K 31/5377; A61K 31/506; C07D 471/04

(52) U.S. Cl. CPC A61K 31/437; C07D 471/04; A61K 31/5377; A61K 31/506
USPC 514/234.2; 546/119; 514/303; 544/127; 544/319; 514/269

(57) ABSTRACT

A compound of formula Ia or formula Ib, or a pharmaceutically acceptable salt or ester thereof, wherein R¹ is selected from: aryl; heteroaryl; —NHR³; fused aryl-C₄₋₇-heterocycloalkyl; —CONR⁴R⁵; —NHCOR⁶; —C₃₋₇-cycloalkyl; -0-C₃₋₇-cycloalkyl; —NR³R⁶; and optionally substituted —C₁₋₆ alkyl; wherein said aryl, heteroaryl, fused aryl-C₄₋₇-heterocycloalkyl and C₄₋₇-heterocycloalkyl are each optionally substituted; Q is CN, halogen, or is selected from C₁₋₆-alkyl, C₃₋₇-cycloalkyl, heterocycloalkyl, aryl and heteroaryl, each of which is optionally substituted with one or more substituents A; R² is selected from hydrogen, aryl, C₁₋₆-alkyl, C₂₋₆-alkenyl, C₃₋₇-cycloalkyl, heteroaryl, C₄₋₇-heterocycloalkyl and halogen, wherein said C₁₋₆-alkyl, Cz-B-alkenyl, aryl, heteroaryl and C₄₋₇-heterocycloalkyl are each optionally substituted; R³ is selected from aryl, heteroaryl, C⁴⁻⁷-heterocycloalkyl, C₃₋₇-cycloalkyl, fused aryl-C-heterocycloalkyl and C₁₋₆-alkyl, each of which is optionally substituted; R⁴ and R⁵ are each independently hydrogen, or optionally substituted C₃₋₇-cycloalkyl, aryl, heteroaryl, C₁₋₆-alkyl or C₃₋₆-heterocycloalkyl; or R⁴ and R⁵ together with the N to which they are attached form a C₃₋₆-heterocycloalkyl ring; each R⁶ is independently selected from C₁₋₆-alkyl, C₃₋₇-cycloalkyl, C-heterocycloalkyl, aryl and heteroaryl, each of which is optionally substituted; each R⁷ is selected from hydrogen, optionally substituted C₁₋₆-alkyl and C₃₋₇-cycloalkyl; each of R⁸ and R⁹ is independently hydrogen or optionally substituted C₁₋₆-alkyl; or R⁸ and R⁹ together with the N to which they are attached form a C₄₋₆-heterocycloalkyl; each R¹⁰ is selected from C₃₋₇-cycloalkyl and optionally substituted C₁₋₆-alkyl; each R¹¹ is independently selected from C₁₋₆-alkyl, C₃₋₇-cycloalkyl, C₁₋₆-alkyl-C₃₋₇-cycloalkyl, C₄₋₇-heterocycloalkyl, aryl and heteroaryl, each of which is optionally substituted; A is selected from halogen, —NR⁴S0₂R⁵, —CN, —OR⁶, —NR⁴R⁵, —NR⁷R¹¹, hydroxyl, —CF₃, —CONR⁴R⁵, —NR⁴COR⁵, —NR⁷(CO)NR⁴R⁵, —N0₂, —C0₂H, —C0₂R⁶, —S0₂R⁶, —S0₂NR⁴R⁵, —NR⁴COR⁵, —NR⁴COOR⁵, ₆-alkyl and —COR⁶. Further aspects relate to pharmaceutical compositions, therapeutic uses and process for preparing compounds of formulae Ia and Ib.

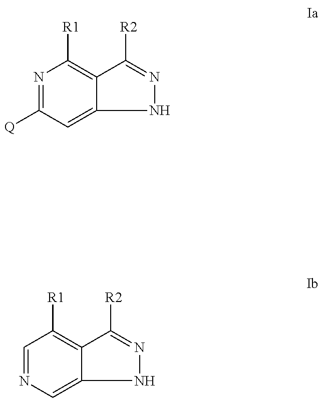

The present invention relates to pyrazolopyridine compounds that are capable of inhibiting one or more kinases, more particularly, LRRK2. The compounds find applications in the treatment of a variety of disorders, including cancer and neurodegenerative diseases such as Parkinson's disease.

### BACKGROUND TO THE INVENTION

There has been much interest raised by the recent discovery that different autosomal dominant point mutations within the gene encoding for LRRK2 predispose humans to develop late-onset PD (OMIM accession number 609007), with a clinical appearance indistinguishable from idiopathic PD [1-3]. The genetic analysis undertaken to date indicates that mutations in LRRK2 are relatively frequent, not only accounting for 5-10% of familial PD, but also being found in a significant proportion of sporadic PD cases [4, 5]. Little is known about how LRRK2 is regulated in cells, what its physiological substrates are and how mutations cause or increase risk of PD.

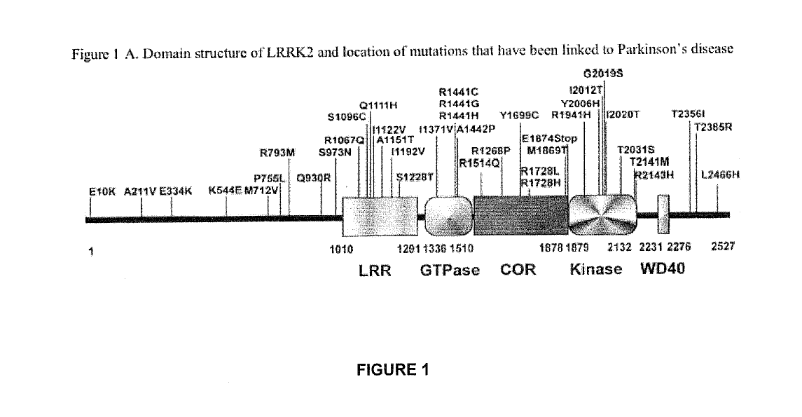

The domain structure of LRRK2 is shown in FIG. 1, which also depicts the mutations that have thus far been reported in patients with PD. The defining feature of the LRRK2 enzyme is a Leucine Rich Repeat (LRR) motif (residues 1010-1291), a Ras-like small GTPase (residues 1336-1510), a region of high amino acid conservation that has been termed the C-terminal Of Ras of complex (COR) domain (residues 1511-1878), a protein kinase catalytic domain (residues 1879-2132) and a C-terminal WD40 motif (2231-2276) [6, 7]. The protein kinase domain of LRRK2 belongs to the tyrosine-like serine/threonine protein kinases and is most similar to the kinase RIP (Receptor Interacting Protein), which play key roles in innate immunity signalling pathways [8]. To date, almost 40 single amino acid substitution mutations have been linked to autosomal-dominant PD and the location of these mutations is illustrated in FIG. 1A ([2, 3]). The most prevalent mutant form of LRRK2 accounting for approximately 6% of familial PD and 3% of sporadic PD cases in Europe, comprises an amino acid substitution of Gly2019 to a Ser residue. Gly2019 is located within the conserved DYG-Mg²⁺-binding motif, in subdomain-VII of the kinase domain [2]. Recent reports suggest that this mutation enhances the autophosphorylation of LRRK2, as well as its ability to phosphorylate myelin basic protein 2-3-fold [9, 10], a finding confirmed by the Applicant [11]. These observations suggest that over-activation of LRRK2 predisposes humans to develop PD, implying that drugs which inhibited LRRK2, could be utilised to halt progression or even perhaps reverse symptoms of some forms of PD.

The study of LRRK2 has been hampered by the difficulty in expressing active recombinant enzyme and by the lack of a robust quantitative assay. In work undertaken by the Applicant, an active recombinant fragment of LRRK2 containing the GTPase-COR and kinase domains encompassing residues 1326-2527 was expressed in 293 cells [11]. The more active G2019S mutant of this LRRK2 fragment was utilised in a KinasE Substrate TRacking and ELucidation (KESTREL) screen in an initial attempt to identify physiological substrates (reviewed in [14]). This led to the identification of a protein termed moesin, which was efficiently phosphorylated by LRRK2 in vitro [11]. Moesin is a member of the Ezrin/Radixin/Moesin (ERM) family of proteins which functions to anchor the actin cytoskeleton to the plasma membrane and plays an important role in regulating membrane structure and organization [15, 16]. It was found that LRRK2 phosphorylated moesin at Thr558 [11], a previously characterised physiologically relevant phosphorylation site [15, 16]. LRRK2 also phosphorylated ezrin and radixin at the equivalent Thr residue. Phosphorylation of ERM proteins at the residue equivalent to Thr558, opens up the structures of these proteins and enables them to interact with actin microfilaments at their C-terminal residues and phosphoinositides and plasma membrane proteins through an N-terminal FERM domain. These findings were utilised to develop a robust and quantitative assay for LRRK2, based upon the phosphorylation of moesin or a short peptide that encompasses the Thr558 residue of moesin which is also efficiently phosphorylated by LRRK2 [11]. These assays were further adapted to develop an improved assay based on the use of the Nictide peptide [17].

The present invention seeks to provide compounds that are capable of inhibiting one or more kinases, more particularly, LRRK, even more preferably LRRK2.

### STATEMENT OF INVENTION

A first aspect of the invention relates to a compound of formula Ia or formula Ib, or a pharmaceutically acceptable salt or ester thereof,

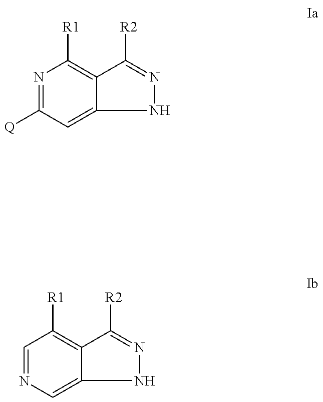

wherein:

R¹ is selected from:

aryl;

heteroaryl;

C₄₋₇-heterocycloalkyl;

—NHR³;

fused aryl-C₄₋₇-heterocycloalkyl;

—CONR⁴R⁵;

—NHCOR⁶;

—C₃₋₇-cycloalkyl;

—NR³R⁶;

OR³;

OH;

NR⁴R⁵; and

—C₁₋₆ alkyl optionally substituted with a substituent selected from R¹¹ and a group A;

wherein said aryl, heteroaryl, fused aryl-C₄₋₇-heterocycloalkyl and C₄₋₇-heterocycloalkyl are each optionally substituted with one or more substituents selected from C₁₋₆-alkyl, C₃₋₇-cycloalkyl, heteroaryl, C₄₋₇-heterocycloalkyl, aryl and a group A, and said C₁₋₆-alkyl, C₃₋₇-cycloalkyl, heteroaryl, C₄₋₇-heterocycloalkyl, and aryl substituents are in turn each optionally substituted with one or more groups selected from R¹¹ and a group A;

R² is selected from hydrogen, aryl, C₁₋₆-alkyl, C₂₋₆-alkenyl, C₃₋₇-cycloalkyl, heteroaryl, C₄₋₇ heterocycloalkyl, fused aryl-C₄₋₇-heterocycloalkyl and halogen, wherein said C″-alkyl, C₂₋₆-alkenyl, aryl, heteroaryl, fused aryl-C₄₋₇-heterocycloalkyl and C₄₋₇-heterocycloalkyl are each optionally substituted with one or more substituents selected from R¹¹ and A;

Q is a halogen, CN, or is selected from C₁₋₆-alkyl, C₃₋₇-cycloalkyl, heterocycloalkyl, aryl and heteroaryl, each of which is optionally substituted with one or more substituents A;

each R³ is selected from aryl, heteroaryl, C₄₋₇-heterocycloalkyl, C₃₋₇-cycloalkyl, fused aryl-C₄₋₇-heterocycloalkyl and C₁₋₆-alkyl, each of which is optionally substituted with one or more substituents selected from R¹¹ and A;

R⁴ and R⁵ are each independently selected from hydrogen, C₃₋₇-cycloalkyl, C₁₋₆-alkyl-C₃₋₇-cycloalkyl, aryl, heteroaryl, C₁₋₆-alkyl and a C₃₋₆-heterocycloalkyl ring optionally further containing one or more groups selected from oxygen, sulfur, nitrogen and CO, and optionally substituted by one or more R¹⁹ groups, wherein each C₁₋₆-alkyl, heteroaryl and aryl is optionally substituted by one or more substituents selected from C₁₋₆-alkyl, halogen, cyano, hydroxyl, aryl, halo-substituted aryl, heteroaryl, —NR⁸R⁹, —NR⁶R⁷, NR⁷(CO)R⁶, —NR⁷COOR⁶, —NR⁷(SO₂)R⁶, —COOR⁶, —CONR⁸R⁹, OR⁶, —SO₂R⁶ and a C₃₋₆-heterocycloalkyl ring optionally further containing one or more groups selected from oxygen, sulfur, nitrogen and CO and optionally substituted by one or more or R¹⁰ groups; or

R⁴ and R⁵ together with the N to which they are attached form a C₃₋₆-heterocycloalkyl ring optionally further containing one or more groups selected from oxygen, sulfur, nitrogen and CO, wherein said C₃₋₆-heterocycloalkyl ring is saturated or unsaturated and is optionally substituted with one or more groups selected from A, NR⁸R⁹ and R¹⁰;

each R⁶ is independently selected from C₁₋₆-alkyl, C₃₋₇ cycloalkyl, C₄₋₇-heterocycloalkyl, aryl and heteroaryl, each of which is optionally substituted by one or more substituents selected from R¹⁰, R¹¹ and A;

each R⁷ is selected from hydrogen, C₁₋₆-alkyl and C₃₋₇-cycloalkyl, wherein said C₁₋₆-alkyl is optionally substituted by one or more halogens;

each of R⁸ and R⁹ is independently selected from hydrogen and C₁₋₆-alkyl, wherein said C₁₋₆-alkyl group is optionally substituted by one or more halogens; or

R⁸ and R⁹ together with the N to which they are attached form a C₄₋₆-heterocycloalkyl ring optionally further containing one or more heteroatoms selected from oxygen and sulfur, wherein said C₄₋₆-heterocycloalkyl ring is optionally substituted by one or more R¹⁰ groups; and

each R¹⁰ is selected from C₃₋₇-cycloalkyl, aryl, heteroaryl, O-heteroaryl, aralkyl and C₁₋₆-alkyl, each of which is optionally substituted by one or more A groups, wherein where R¹⁶ is C₁₋₆-alkyl and two or more R¹⁰ groups are attached to the same carbon atom, the R¹⁰ groups may be linked to form a spiroalkyl group; and

each R¹¹ is independently selected from C₁₋₆-alkyl, C₃₋₇-cycloalkyl, C₁₋₆-alkyl-heteroaryl, C₄₋₇-heterocycloalkyl, aryl and heteroaryl, each of which is optionally substituted with one or more substituents selected from A; and

A is selected from halogen, —NR⁴SO₂R⁵, —CN, —NR⁴R⁵, —W⁷R¹¹, hydroxyl, —CF₃, —CONR⁴R⁵, —NR⁴COR⁵, —NR⁷(CO)NR⁴R⁵, —NO₂, —CO₂H, —CO₂R⁶, —SO₂R⁶, —SO₂NR⁴R⁵, —NR⁴COR⁵, —NR⁴COOR⁵, C₁₋₆-alkyl, aryl and —COR⁶.

A second aspect of the invention relates to a pharmaceutical composition comprising at least one compound as described above and a pharmaceutically acceptable carrier, diluent or excipient.

A third aspect of the invention relates to a compound as described above for use in medicine.

A fourth aspect of the invention relates to a compound as described above for use in treating a disorder selected from cancer and neurodegenerative diseases such as Parkinson's Disease.

A fifth aspect of the invention relates to the use of a compound as described above in the preparation of a medicament for treating or preventing a disorder selected from cancer and neurodegenerative diseases such as Parkinson's Disease.

A sixth aspect of the invention relates to the use of a compound as described above in the preparation of a medicament for the prevention or treatment of a disorder caused by, associated with or accompanied by any abnormal kinase activity wherein the kinase is preferably LRRK, more preferably LRRK2.

A seventh aspect of the invention relates to a method of treating a mammal having a disease state alleviated by inhibition of a kinase (preferably LRRK, more preferably LRRK2), wherein the method comprises administering to a mammal a therapeutically effective amount of a compound as described above.

An eighth aspect of the invention relates to the use of a compound as described above in an assay for identifying further candidate compounds capable of inhibition of a kinase, preferably LRRK, more preferably LRRK2.

A ninth aspect of the invention relates to processes for preparing compounds of formula Ia and formula Ib.

### DETAILED DESCRIPTION

The present invention relates to pyrazolopyridine compounds that are capable of inhibiting one or more kinases, more particularly LRRK, even more particularly LRRK2. Specifically, the invention relates to substituted pyrazolo[4,3-c]pyridine derivatives.

“Alkyl” is defined herein as a straight-chain or branched alkyl radical, for example, methyl, ethyl, propyl, isopropyl, butyl, isobutyl, tert-butyl, pentyl, hexyl.

“Cycloalkyl” is defined herein as a monocyclic alkyl ring, such as, cyclopropyl, cyclobutyl, cyclopentyl, cyclohexyl or cycloheptyl, or a fused bicyclic ring system such as norbornane.

“Halogen” is defined herein as chloro, fluoro, bromo or iodo.

As used herein, the term “aryl” refers to a C₆₋₁₂ aromatic group, which may be benzocondensed, for example, phenyl or naphthyl.

“Heteroaryl” is defined herein as a monocyclic or bicyclic C₂₋₁₂ aromatic ring comprising one or more heteroatoms (that may be the same or different), such as oxygen, nitrogen or sulphur. Examples of suitable heteroaryl groups include thienyl, furanyl, pyrrolyl, pyridinyl, oxazolyl, thiazolyl, imidazolyl, pyrazolyl, isoxazolyl, isothiazolyl, oxadiazolyl, triazolyl, thiadiazolyl etc. and benzo derivatives thereof, such as benzofuranyl, benzothienyl, benzimidazolyl, indolyl, isoindolyl, indazolyl etc.; or pyridyl, pyrazinyl, pyrimidinyl, pyridazinyl, triazinyl etc. and benzo derivatives thereof, such as quinolinyl, isoquinolinyl, cinnolinyl, phthalazinyl, quinazolinyl, quinoxalinyl, naphthyridinyl etc.

“Heterocycloalkyl” refers to a cyclic aliphatic group containing one or more heteroatoms selected from nitrogen, oxygen and sulphur, which is optionally interrupted by one or more —(CO)— groups in the ring and/or which optionally contains one or more double bonds in the ring. Preferably, the heterocycloalkyl group is a C₃₋₇-heterocycloalkyl, more preferably a Cₘ-heterocycloalkyl. Alternatively, the heterocycloalkyl group is a C₄₋₇-heterocycloalkyl, more preferably a C₄₋₅-heterocycloalkyl. Preferred heterocycloalkyl groups include, but are not limited to, piperazinyl, piperidinyl, morpholinyl, thiomorpholinyl, pyrrolidinyl, tetrahydrofuranyl and tetrahydropyranyl.

In one preferred embodiment, the invention relates to compounds of formula Ia.

In another preferred embodiment, the invention relates to compounds of formula Ib.

The preferred definitions set forth below apply to formula Ia and formula Ib

In one preferred embodiment of the invention, R² is selected from:

hydrogen;

halogen, more preferably bromine;

aryl optionally substituted by one or more substituents selected from R¹¹ and A;

C₁₋₅-alkyl optionally substituted by one or more substituents selected from R¹¹ and A;

C₂₋₆-alkenyl optionally substituted by one or more A substituents;

C₃₋₇-cycloalkyl;

heteroaryl optionally substituted by one or more substituents selected from R¹¹ and A;

C₄₋₇-heterocycloalkyl; and

fused aryl-C₄₋₇-heterocycloalkyl.

In one preferred embodiment of the invention, R² is selected from:

aryl optionally substituted by one or more substituents selected from —NR⁴COR⁵, —CONR⁴R⁵, OR⁶, halogen, optionally substituted C₁₋₆-alkyl, CN, C₄₋₇-heterocycloalkyl and heteroaryl;

C₁₋₆-alkyl optionally substituted by one or more substituents selected from —NR⁴COR⁵, —CONR⁴R⁵, —NR⁴R⁵, OR⁶, optionally substituted aryl, optionally substituted heteroaryl and C₄₋₇-heterocycloalkyl;

C₂₋₆-alkenyl optionally substituted by one or more —CONR⁴R⁵ substituents;

C₃₋₇-cycloalkyl;

heteroaryl optionally substituted by one or more substituents selected from C₄₋₇-heterocycloalkyl, C₁₋₆-alkyl, C₃₋₇-cycloalkyl, C₁₋₆-alkyl-C₃₋₇-cycloalkyl and OR⁶;

C₄₋₇-heterocycloalkyl; and

fused aryl-C₄₋₇-heterocycloalkyl.

In one preferred embodiment of the invention, R² is selected from:

a phenyl group optionally substituted by one or more substituents selected from —NHCO—C₁₋₆-alkyl, CO—(N-morpholinyl), Cl, F, —OC₁₋₆-alkyl, —CONMe₂, OCF₃, CN, CF₃, C₁₋₆-alkyl-(A), N-morpholinyl and pyrazolyl;

a heteroaryl group selected from pyridinyl, quinolinyl, pyrazoyl, furanyl and pyrimidinyl, each of which may be optionally substituted by one or more substituents selected from C₁₋₆-alkyl, aralkyl, OC₁₋₆-alkyl, N-morpholinyl;

a C₁₋₆-alkyl group optionally substituted by one or more substituents selected from —CONR⁴R⁵, phenyl, pyridinyl and oxadiazolyl and piperidinyl, wherein said phenyl, pyridinyl and oxadiazolyl and piperidinyl groups are each optionally further substituted by one or more —NR⁴COR⁵, —CONR⁴R⁵, COR⁶, SO₂R⁶ or aryl groups.

In a more preferred embodiment of the invention, each —CONR⁴R⁵ group is independently selected from:

—CO(N-morpholinyl), —CO(N-piperidinyl), —CO(N-pyrrolidinyl), —CO—(N-piperazinyl), each of which may be optionally further substituted by one or more substituents selected from aryl, heteroaryl, CF₃, aralkyl, —NR⁴COR⁵—CONR⁴R⁵, —NR⁴R⁵, halogen, C₁₋₆-alkyl; and —CON(C₁₋₆-alkyl)₂, CONH(C₁₋₆-alkyl), CON(C₁₋₆-alkyl)(aralkyl), CONH(C₃₋₇-cycloalkyl), —CONH(aryl), —CONH(heteroaryl), wherein said C₁₋₆-alkyl, aralkyl, aryl and heteroaryl groups are each optionally further substituted by one or more R¹¹ or A groups.

In one preferred embodiment of the invention, R² is a C₁₋₆-alkyl group optionally substituted by one or more substituents selected from —NR⁴COR⁵, —CONR⁴R⁵, —NR⁴R⁵, OR⁶, C₄₋₇-heterocycloalkyl, heteroaryl and aryl, wherein said aryl group is optionally substituted by one or more substituents selected from —NR⁴COR⁵ and —CONR⁴R⁵.

In one preferred embodiment of the invention, R² is selected from —CH₂CH₂CO—NR⁴R⁵, C₁₋₆-alkyl, C₃₋₇ cycloalkyl and a heteroaryl selected from furanyl and pyrazolyl, wherein said furanyl and pyrazolyl groups may be optionally substituted by one or more substituents selected from C₁₋₆-alkyl, C₃₋₇-cycloalkyl and C₁₋₆-alkyl-C₃₋₇-cycloalkyl.

In one preferred embodiment of the invention, R² is selected from Me,

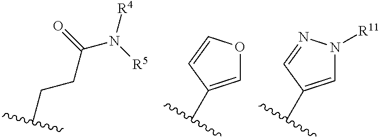

wherein R⁴ and R⁵ together with the N to which they are attached form a C₃₋₆-heterocycloalkyl ring optionally further containing one or more groups selected from oxygen, sulfur, nitrogen and CO, wherein said C₃₋₆-heterocycloalkyl ring is saturated or unsaturated and is optionally substituted with one or more groups selected from A, NR⁸R⁹ and R¹⁰. Even more preferably, R⁴ and R⁵ together with the N to which they are attached form a 6-membered heterocycloalkyl ring that is optionally substituted with one or more groups selected from A, NR⁸R⁹ and R¹⁰. More preferably still, R⁴ and R⁵ together with the N to which they are attached form a saturated 6-membered ring (more preferably, a piperidinyl ring) that is optionally substituted with one or more groups selected from A, NR⁸R⁹ and R¹⁰.

In one highly preferred embodiment of the invention, R² is selected from Me,

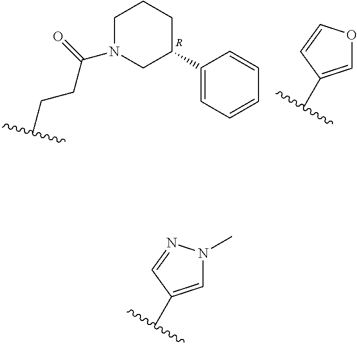

In one preferred embodiment of the invention, R² is selected from aryl, C″-alkyl and heteroaryl, each of which is optionally substituted with one or more substituents selected from R¹¹ and A.

In another preferred embodiment of the invention, R² is selected from aryl, C″-alkyl and heteroaryl, each of which is optionally substituted with one or more substituents selected from CONR⁴R⁵, CF₃, C″-alkyl, OR⁶ and C₄₋₇-heterocycloalkyl.

In another preferred embodiment of the invention, R² is selected from C″-alkyl, phenyl, pyridinyl, pyrimidinyl, pyrazolyl, each of which is optionally substituted by one or more substituents selected from CONR⁴R⁵, CF₃, C″-alkyl, OR⁶ and C₄₋₇-heterocycloalkyl.

In another preferred embodiment of the invention, R² is selected from C″-alkyl, phenyl, pyridinyl, pyrimidinyl and pyrazolyl, each of which is optionally substituted by one or more substituents selected from CONMe₂, CF₃, iso-butyl, iso-propyl, OEt and morpholinyl.

In a highly preferred embodiment of the invention, R² is selected from the following: Me

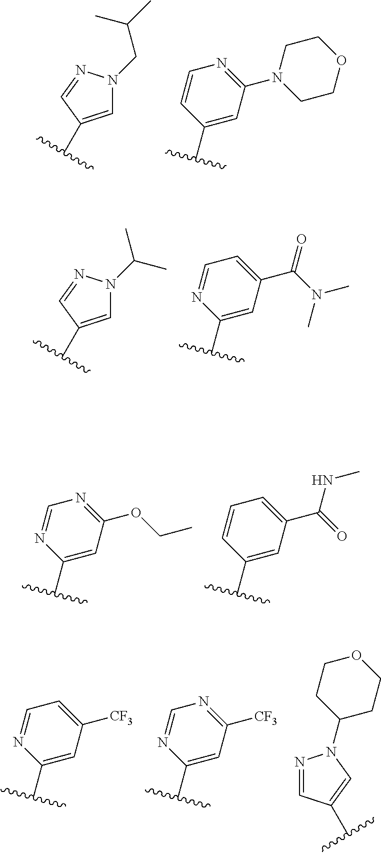

In one preferred embodiment of the invention, R² is an unsubstituted C₁₋₆-alkyl group, more preferably methyl.

In one preferred embodiment of the invention, R¹ is selected from:

—NHR³;

aryl;

heteroaryl;

C₄₋₇-heterocycloalkyl;

fused aryl-C₄₋₇-heterocycloalkyl;

—C₃₋₇-cycloalkyl;

—NR³R⁶;

OR³;

NR⁴R⁵; and

—C₁₋₆ alkyl optionally substituted with a substituent selected from R¹¹ and a group A;

wherein said aryl, heteroaryl, fused aryl-C₄₋₇-heterocycloalkyl and C₄₋₇-heterocycloalkyl are each optionally substituted with one or more substituents selected from C₁₋₆-alkyl, C₃₋₇-cycloalkyl, heteroaryl, C₄₋₇-heterocycloalkyl, aryl and a group A, and said C₁₋₆-alkyl, C₃₋₇-cycloalkyl, heteroaryl, C₄₋₇-heterocycloalkyl, and aryl substituents are in turn each optionally substituted with one or more groups selected from R¹¹ and a group A.

In one preferred embodiment of the invention, R¹ is —NHR³ and R³ is selected from: C₁₋₆-alkyl, optionally substituted by one or more —OR⁶, NR⁴COR⁵, heteroaryl, aryl, C₄₋₇-heterocycloalkyl, and C₃₋₇-cycloalkyl groups, wherein said aryl and heteroaryl groups are each independently optionally further substituted by one or more groups selected from CF₃, halogen, C₁₋₆-alkyl, —OR⁶ and —NR⁴R⁵;

a phenyl group optionally substituted by one or more substituents selected from —OR⁶, NR⁴COR⁵, —CONR⁴R⁵, aryl, —NR⁴R⁵, C₁₋₆-alkyl-heteroaryl, heteroaryl, halogen, —SO₂R⁶, CN, CF₃, C₁₋₆-alkyl, —SO₂NR⁴R⁵, —NR⁴SO₂R⁵, wherein said C₁₋₆-alkyl, heteroaryl and aryl groups are each independently optionally further substituted by one or more groups selected from CN, CF₃, halogen, C₁₋₆-alkyl, —OR⁶ and —NR⁴R⁵;

a heteroaryl group optionally substituted by one or more substituents selected from aryl, C₁₋₆-alkyl, and —NR⁴R⁵, wherein said aryl group is optionally further substituted by one or more A groups;

a C₄₋₇-heterocycloalkyl optionally substituted by one or more —COR⁶ groups;

a C₃₋₇-cycloalkyl group optionally substituted by one or more halogen or C″-alkyl groups.

In one preferred embodiment of the invention, R¹ is —NHR³, wherein R³ is selected from C₁₋₆-alkyl, C₃₋₇-cycloalkyl, C₄₋₇-heterocycloalkyl and aryl, each of which may be optionally substituted by one or more with one or more substituents selected from R¹¹ and A.

In one preferred embodiment of the invention, R¹ is —OR³, wherein R³ is selected from C₁₋₆-alkyl, C₃₋₇-cycloalkyl, C₄₋₇-heterocycloalkyl and aryl, each of which may be optionally substituted by one or more with one or more substituents selected from R¹¹ and A.

In one preferred embodiment of the invention, R¹ is —OR³, wherein R³ is C₁₋₆-alkyl, C₃₋₇-cycloalkyl or C₄₋₇-heterocycloalkyl, each of which may be optionally substituted by one or more A substituents. In one particularly preferred embodiment of the invention, R¹ is —O—C₃₋₇-cycloalkyl, more preferably, —O-cyclohexyl.

In one preferred embodiment of the invention, R¹ is selected from heteroaryl, —NHR³ and OR³, wherein said heteroaryl group is optionally substituted with one or more substituents seleted from the group A.

In one preferred embodiment of the invention, R¹ is aryl or heteroaryl, each of which may be optionally substituted by one or more with one or more substituents selected from R¹¹ and A, more preferably R¹ is furyl.

In one preferred embodiment of the invention, R¹ is —NH—C₃₋₇-cycloalkyl or NH—C₄₋₇-heterocycloalkyl, each of which may be optionally substituted by one or more A substituents.

Preferably, for all embodiments, A is halogen or C₁₋₅-alkyl.

In one preferred embodiment of the invention, R³ is cyclohexyl or tetrahydropyranyl, each of which may be optionally substituted by one or more A substituents.

In one preferred embodiment of the invention, R¹ is selected from the following:

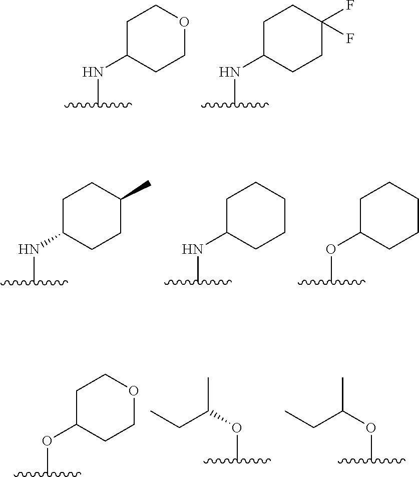

In one preferred embodiment of the invention, R¹ is —OR³ or NHR³, and R³ is cyclohexyl, Me or tetrahydropyran-4-yl.

In one preferred embodiment of the invention, R¹ is —NH-cyclohexyl.

In one preferred embodiment of the invention, R¹ is —NHR³ and R² is an unsubstituted C₁₋₆-alkyl group, more preferably methyl.

In one preferred embodiment of the invention, R¹ is —NHR³ and R² is a C″-alkyl group substituted by one or more —CONR⁴R⁵ groups.

In one preferred embodiment of the invention, R¹ is —NHR³ and R² is an aryl or heteroaryl group, each of which may be optionally substituted by one or more substituents selected from C₄₋₇-heterocycloalkyl, C₁₋₆-alkyl, C₃₋₇-cycloalkyl, C₁₋₅-alkyl-C₃₋₇-cycloalkyl and OR⁶.

In one preferred embodiment of the invention, R¹ is —OR³ and R² is a C₁₋₆-alkyl group, more preferably methyl.

In one preferred embodiment of the invention, R¹ is selected from:

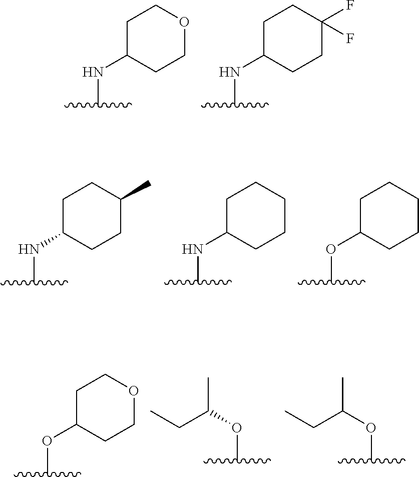

and R² is selected from

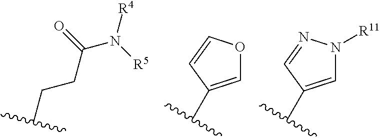

wherein R⁴ and R⁵ together with the N to which they are attached form a C₃₋₆-heterocycloalkyl ring optionally further containing one or more groups selected from oxygen, sulfur, nitrogen and CO, wherein said C₃₋₆-heterocycloalkyl ring is saturated or unsaturated and is optionally substituted with one or more groups selected from A, NR⁸R⁹ and R¹⁰. Even more preferably, R⁴ and R⁵ together with the N to which they are attached form a 6-membered heterocycloalkyl ring that is optionally substituted with one or more groups selected from A, NR⁸R⁹ and R¹⁰. More preferably still, R⁴ and R⁵ together with the N to which they are attached form a saturated 6-membered ring (more preferably, a piperidinyl ring) that is optionally substituted with one or more groups selected from A, NR⁸R⁹ and R¹⁰.

More preferably, R¹ is as defined above, and R² is selected from Me,

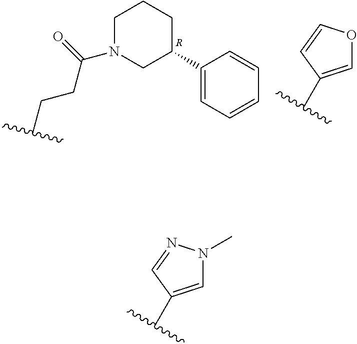

In one preferred embodiment of the invention:

R¹ is selected from aryl, heteroaryl, C₄₋₇-heterocycloalkyl, fused aryl-C₄₋₇-heterocycloalkyl and —NHR³, wherein said aryl, heteroaryl, fused aryl-C₄₋₇-heterocycloalkyl and C₄₋₇-heterocycloalkyl are each optionally substituted with one or more substituents selected from C₁₋₆-alkyl, heteroaryl, C₄₋₇-heterocycloalkyl, aryl and a group A, and said C₁₋₆-alkyl, C₃₋₇-cycloalkyl, heteroaryl, C₄₋₇-heterocycloalkyl, and aryl substituents are in turn each optionally substituted with one or more groups selected from R¹¹ and a group A; and

R² is selected from hydrogen, aryl, C₁₋₆-alkyl, C₃₋₇-cycloalkyl, heteroaryl, C₄₋₇ heterocycloalkyl and halogen, wherein said C₁₋₆-alkyl, aryl, heteroaryl and C₄₋₇-heterocycloalkyl are each optionally substituted with one or more substituents selected from R¹¹ and A.

In another preferred embodiment of the invention R² is a C₁₋₆-alkyl group optionally substituted with one or more substituents selected from R¹¹ and A.

In one preferred embodiment of the invention R¹ is selected from: NH—R³, where R³ is selected from C₁₋₆-alkyl, morpholinyl, C₃₋₇-cycloalkyl, fused aryl-C₄₋₇-heterocycloalkyl, piperidinyl, tetrahydropyranyl, piperazinyl, phenyl, pyridinyl, indazolyl and pyrazolyl, each of which is optionally substituted by one or more substituents selected from R¹¹ and A; and

furyl, pyrazolyl and phenyl, each of which is optionally substituted by one or more substituents selected from R¹¹ and A.

In one preferred embodiment of the invention R¹ is selected from:

NH—C₁₋₆-alkyl, wherein said C₁₋₆-alkyl is optionally substituted by one or more substituents selected from OR⁶, OH, C₄₋₇ heterocycloalkyl, NR⁴R⁵, heteroaryl, C₃₋₇-cycloalkyl, phenyl, wherein said phenyl group is optionally substituted by one or more halo groups, and said C₄₋₇ heterocycloalkyl group is optionally substituted by one or more C₁₋₆-alkyl groups; NH-piperazinyl, wherein said piperazinyl is optionally substituted by one or more substituents selected from C₁₋₆-alkyl, aryl, C₁₋₆-alkyl-aryl and heteroaryl, each of which is optionally further substituted by one or more halo groups;

NH-morpholinyl;

NH—C₃₋₇-cycloalkyl, wherein said C₃₋₇-cycloalkyl is optionally substituted by one or more substituents selected from OH and halo;

NH-fused aryl-C₄₋₇-heterocycloalkyl, wherein said fused aryl-C₄₋₇-heterocycloalkyl is optionally substituted by one or more C₁₋₆-alkyl groups;

NH-piperidinyl, wherein said piperidinyl is optionally substituted by one or more C₁₋₆-alkyl groups;

NH-tetrahydropyranyl;

a furyl group;

a pyrazolyl group, optionally substituted by one or more C₁₋₆-alkyl groups;

NH-phenyl, wherein said phenyl is optionally substituted by one or more substituents selected from halo, CF₃, OH, OR⁶, NR⁴SO₂R⁵, NR⁴R⁵, C₄₋₇ heterocycloalkyl, CONR⁴R⁵ and —NR⁴COR⁵;

NH-pyridinyl, wherein said pyridinyl is optionally substituted by one or more substituents selected from C₄₋₇ heterocycloalkyl and aryl, wherein said aryl group is optionally further substituted with one or more halo groups;

phenyl, optionally substituted by one or more substituents selected from halo, OR⁶, —NR⁴SO₂R⁵, CN, C₄₋₇ heterocycloalkyl and C₁₋₆-alkyl-NR⁴SO₂R⁵;

NH-indazolyl, wherein said indazolyl is optionally substituted by one or more C₁₋₆-alkyl groups; and

NH-pyrazolyl.

In one preferred embodiment of the invention R¹ is selected from: NH—C₁₋₆-alkyl, wherein said C₁₋₆-alkyl is optionally substituted by one or more substituents selected from OMe, OH, tetrahydropyranyl, pyrrolidinyl, NEt₂, imidazolyl, cyclopropyl, cyclobutyl, cyclopentyl, cyclohexyl, phenyl, wherein said phenyl group is optionally substituted by one or more chloro groups, and said pyrrolidinyl group is optionally substituted by one or more methyl groups;

NH-piperazinyl, wherein said piperazinyl is optionally substituted by one or more substituents selected from methyl, phenyl, CH₂-phenyl and pyridinyl, wherein the phenyl group is optionally further substituted by one or more F or Cl groups;

NH-morpholinyl;

NH-cyclopropyl, NH-cyclobutyl, NH-cyclopentyl and NH-cyclohexyl, wherein said cyclopropyl, cyclobutyl, cyclopentyl and cyclohexyl groups are optionally substituted by one or more substituents selected from OH and F;

NH-(1,2,3,4-tetrahydroisoquinolinyl), wherein said 1,2,3,4-tetrahydroisoquinolinyl group is optionally substituted by one or more methyl groups;

NH-piperidinyl, wherein said piperidinyl is optionally substituted by one or more methyl groups;

NH-tetrahydropyranyl;

a furyl group;

a pyrazolyl group, optionally substituted by one or more methyl groups;

NH-phenyl, wherein said phenyl is optionally substituted by one or more substituents selected from F, Cl, Br, CF₃, OH, OEt, NHSO₂Me, NMe₂, morpholinyl, CONMe₂, CONH₂ and —NHCOMe;

NH-pyridinyl, wherein said pyridinyl is optionally substituted by one or more substituents selected from morpholinyl and phenyl wherein said phenyl group is optionally further substituted with one or more CN groups;

phenyl, optionally substituted by one or more substituents selected from F, Cl, OMe, —NHSO₂Me, CN, morpholinyl and CH₂—NHSO₂Me;

NH-indazolyl, wherein said indazolyl is optionally substituted by one or more methyl groups; and

NH-pyrazolyl.

In one preferred embodiment of the invention, Q is selected from a halogen, CN, C₁₋₆-alkyl, C₃₋₇-cycloalkyl, and C₄₋₇-heterocycloalkyl and heteroaryl, wherein said C₁₋₆-alkyl, C₃₋₇-cycloalkyl, C₄₋₇-heterocycloalkyl and heteroaryl are each independently optionally substituted with one or more substituents from the group A. Preferably, A is halo or C₁₋₆-alkyl.

In a more preferred embodiment of the invention, Q is selected from CN, cyclopropyl, CF₃, chloro, methyl, N-morpholinyl and 1-methylpyrazol-4-yl.

In one preferred embodiment, Q is a halogen, or is selected from C₁₋₆-alkyl, heterocycloalkyl and heteroaryl, each of which is optionally substituted with one or more substituents A.

More preferably, Q is selected from chloro, methyl, N-morpholinyl and 1-methylpyrazol-4-yl.

In one highly preferred embodiment of the invention the compound of formula Ia or formula Ib is selected from the following:

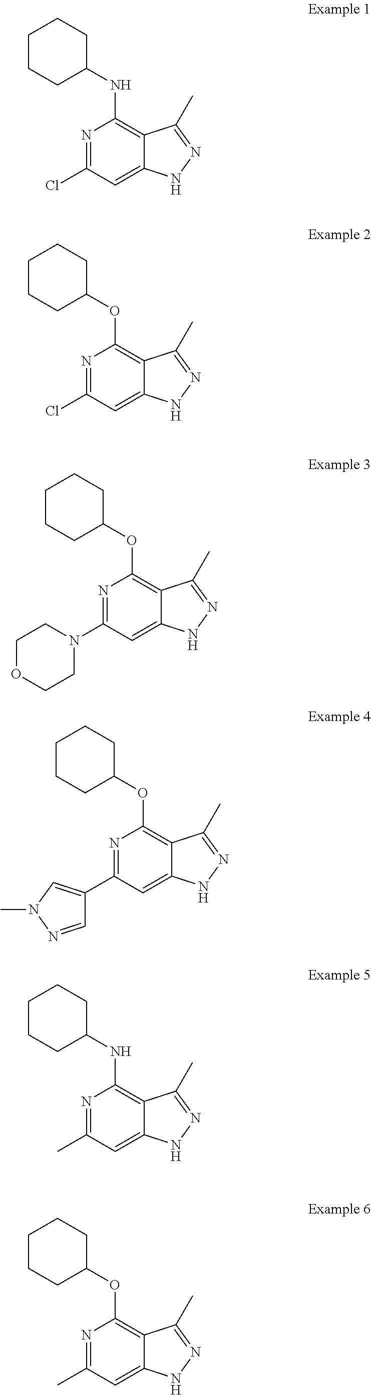 [SMILES: Cc1n[nH]c2cc(Cl)nc(NC3CCCCC3)c12 ; Cc1n[nH]c2cc(Cl)nc(OC3CCCCC3)c12 ; Cc1n[nH]c2cc(N3CCOCC3)nc(OC3CCCCC3)c12 ; Cc1n[nH]c2cc(-c3cnn(C)c3)nc(OC3CCCCC3)c12 ; Cc1cc2[nH]nc(C)c2c(NC2CCCCC2)n1 ; Cc1cc2[nH]nc(C)c2c(OC2CCCCC2)n1]

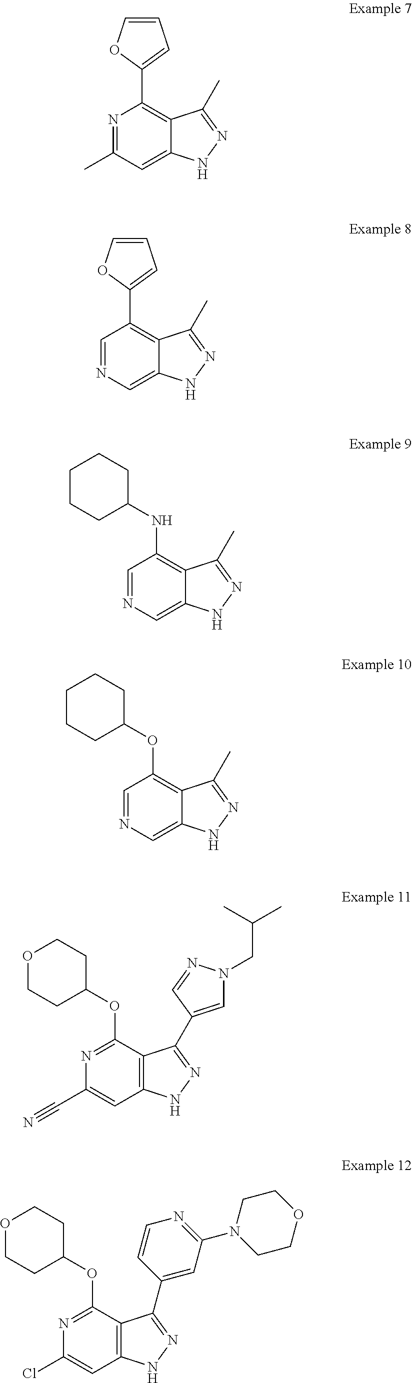 [SMILES: Cc1cc2[nH]nc(C)c2c(-c2ccco2)n1 ; Cc1n[nH]c2cncc(-c3ccco3)c12 ; Cc1n[nH]c2cncc(NC3CCCCC3)c12 ; Cc1n[nH]c2cncc(OC3CCCCC3)c12 ; CC(C)Cn1cc(-c2n[nH]c3cc(C#N)nc(OC4CCOCC4)c23)cn1 ; Clc1cc2[nH]nc(-c3ccnc(N4CCOCC4)c3)c2c(OC2CCOCC2)n1]

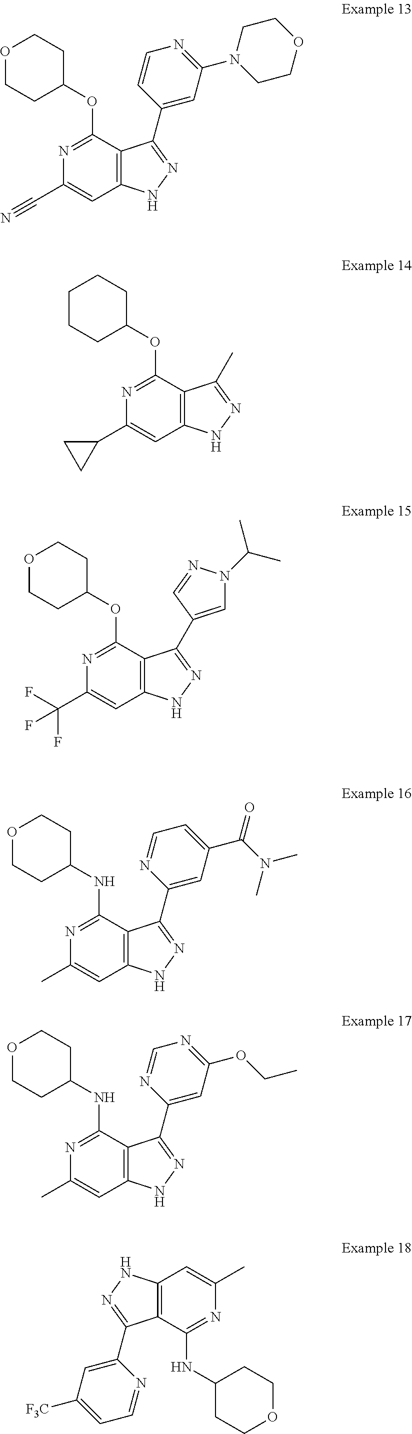 [SMILES: N#Cc1cc2[nH]nc(-c3ccnc(N4CCOCC4)c3)c2c(OC2CCOCC2)n1 ; Cc1n[nH]c2cc(C3CC3)nc(OC3CCCCC3)c12 ; CC(C)n1cc(-c2n[nH]c3cc(C(F)(F)F)nc(OC4CCOCC4)c23)cn1 ; Cc1cc2[nH]nc(-c3cc(C(=O)N(C)C)ccn3)c2c(NC2CCOCC2)n1 ; CCOc1cc(-c2n[nH]c3cc(C)nc(NC4CCOCC4)c23)ncn1 ; Cc1cc2[nH]nc(-c3cc(C(F)(F)F)ccn3)c2c(NC2CCOCC2)n1]

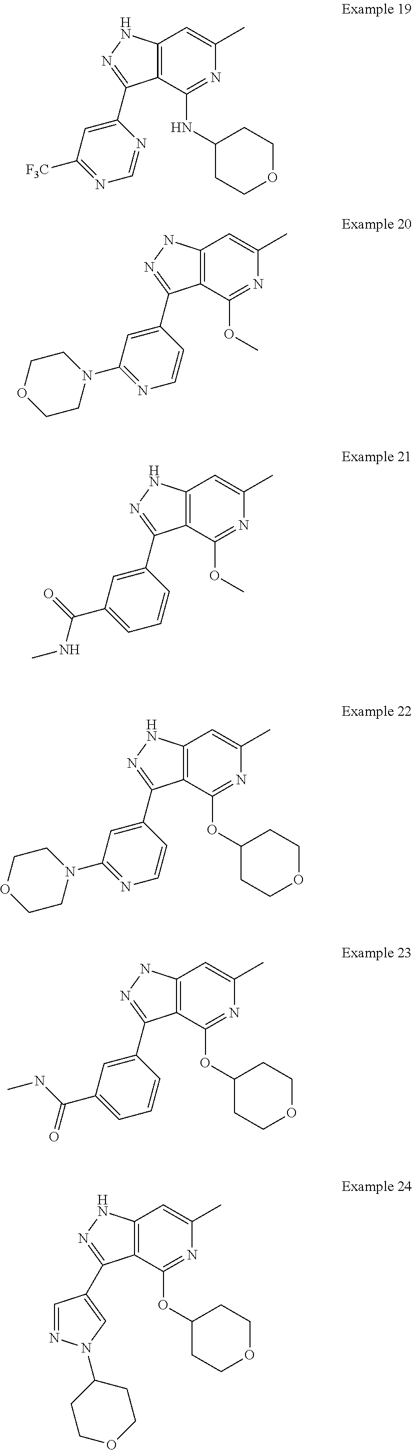

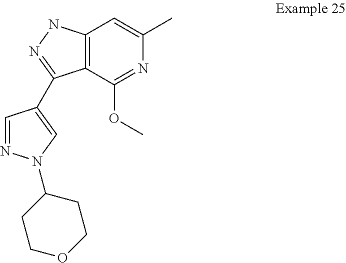 [SMILES: COc1nc(C)cc2[nH]nc(-c3cnn(C4CCOCC4)c3)c12]

or a pharmaceutically acceptable salt thereof.

### Biological Activity

The compounds of the present invention are capable of inhibiting one or more kinases, preferably, LRRK, even more preferably LRRK2.

In one preferred embodiment, the compound of the invention is capable of inhibiting LRRK2, as measured by the assay described in the accompanying Examples section. Preferably, the compound of the invention exhibits an IC₅₀ value of less than 10 μM, more preferably less than 5 μM, even more preferably less than 1 μM or less than 0.5 less μM, more preferably still less than 0.1 μM.

Preferably, the compound of the invention exhibits a KI value of less than 10 μM, more preferably less than 5 μM, even more preferably less than 1 μM or less than 0.5 less μM, more preferably still less than 0.1 μM.

Particularly preferred compounds include the following: [1], [2], [6]-[10] and [1]-[25].

Highly preferred compounds include the following: [11], [12], [15], [17], [18, [19], [22], [24] and [25].

### Therapeutic Applications

A further aspect of the invention relates to a compound as described above for use in medicine.

Another aspect of the invention relates to a compound as described above for use in treating cancer or a neurodegenerative disorder.

Another aspect relates to the use of a compound as described above in the preparation of a medicament for treating or preventing a neurodegenerative disorder. Preferably, the neurodegenerative disorder is Parkinson's Disease.

Another aspect relates to the use of a compound as described above in the preparation of a medicament for treating or preventing a proliferative disorder, for example, cancer.

Preferably, the compound is administered in an amount sufficient to inhibit one or more kinases, preferably LRRK, even more preferably LRRK2.

Yet another aspect relates to the use of a compound of the invention in the preparation of a medicament for the prevention or treatment of a disorder caused by, associated with or accompanied by any abnormal activity against a biological target, wherein the target is a kinase, more preferably LRRK, even more preferably LRRK2.

Preferably, the disorder is Parkinson's Disease.

Another aspect of the invention relates to a method of treating a protein kinase related disease or disorder. The method according to this aspect of the present invention is effected by administering to a subject in need thereof a therapeutically effective amount of a compound of the present invention, as described hereinabove, either per se, or, more preferably, as a part of a pharmaceutical composition, mixed with, for example, a pharmaceutically acceptable carrier, as is detailed hereinafter.

Yet another aspect of the invention relates to a method of treating a mammal having a disease state alleviated by inhibition of a protein kinase, wherein the method comprises administering to a mammal a therapeutically effective amount of a compound according to the invention.

Preferably, the disease state is alleviated by the inhibition of the protein kinase LRRK, more preferably LRRK2.

Preferably, the mammal is a human.

The term “method” refers to manners, means, techniques and procedures for accomplishing a given task including, but not limited to, those manners, means, techniques and procedures either known to, or readily developed from known manners, means, techniques and procedures by practitioners of the chemical, pharmacological, biological, biochemical and medical arts.

The term “administering” as used herein refers to a method for bringing a compound of the present invention and a protein kinase together in such a manner that the compound can affect the enzyme activity of the protein kinase either directly; i.e., by interacting with the protein kinase itself or indirectly; i.e., by interacting with another molecule on which the catalytic activity of the protein kinase is dependent. As used herein, administration can be accomplished either in vitro, i.e. in a test tube, or in vivo, i.e., in cells or tissues of a living organism.

Herein, the term “treating” includes abrogating, substantially inhibiting, slowing or reversing the progression of a disease or disorder, substantially ameliorating clinical symptoms of a disease or disorder or substantially preventing the appearance of clinical symptoms of a disease or disorder.

Herein, the term “preventing” refers to a method for barring an organism from acquiring a disorder or disease in the first place.

The term “therapeutically effective amount” refers to that amount of the compound being administered which will relieve to some extent one or more of the symptoms of the disease or disorder being treated.

For any compound used in this invention, a therapeutically effective amount, also referred to herein as a therapeutically effective dose, can be estimated initially from cell culture assays. For example, a dose can be formulated in animal models to achieve a circulating concentration range that includes the IC₅₀ or the IC₁₀₀ as determined in cell culture. Such information can be used to more accurately determine useful doses in humans. Initial dosages can also be estimated from in vivo data. Using these initial guidelines one of ordinary skill in the art could determine an effective dosage in humans.

Moreover, toxicity and therapeutic efficacy of the compounds described herein can be determined by standard pharmaceutical procedures in cell cultures or experimental animals, e.g., by determining the LD₅₀ and the ED₅₀. The dose ratio between toxic and therapeutic effect is the therapeutic index and can be expressed as the ratio between LD₅₀ and ED₅₀. Compounds which exhibit high therapeutic indices are preferred. The data obtained from these cell cultures assays and animal studies can be used in formulating a dosage range that is not toxic for use in human. The dosage of such compounds lies preferably within a range of circulating concentrations that include the ED₅₀ with little or no toxicity. The dosage may vary within this range depending upon the dosage form employed and the route of administration utilized. The exact formulation, route of administration and dosage can be chosen by the individual physician in view of the patient's condition. (see, e.g., Fingl et al, 1975, In: The Pharmacological Basis of Therapeutics, chapter 1, page 1).

Dosage amount and interval may be adjusted individually to provide plasma levels of the active compound which are sufficient to maintain therapeutic effect. Usual patient dosages for oral administration range from about 50-2000 mg/kg/day, commonly from about 100-1000 mg/kg/day, preferably from about 150-700 mg/kg/day and most preferably from about 250-500 mg/kg/day. Preferably, therapeutically effective serum levels will be achieved by administering multiple doses each day. In cases of local administration or selective uptake, the effective local concentration of the drug may not be related to plasma concentration. One skilled in the art will be able to optimize therapeutically effective local dosages without undue experimentation.

As used herein, “kinase related disease or disorder” refers to a disease or disorder characterized by inappropriate kinase activity or over-activity of a kinase as defined herein. Inappropriate activity refers to either; (i) kinase expression in cells which normally do not express said kinase; (ii) increased kinase expression leading to unwanted cell proliferation, differentiation and/or growth; or, (iii) decreased kinase expression leading to unwanted reductions in cell proliferation, differentiation and/or growth. Over-activity of kinase refers to either amplification of the gene encoding a particular kinase or production of a level of kinase activity, which can correlate with a cell proliferation, differentiation and/or growth disorder (that is, as the level of the kinase increases, the severity of one or more of the symptoms of the cellular disorder increases). Over activity can also be the result of ligand independent or constitutive activation as a result of mutations such as deletions of a fragment of a kinase responsible for ligand binding.

Preferred diseases or disorders that the compounds described herein may be useful in preventing, include cancer and neurodegenerative disorders such as Parkinson's Disease.

Thus, the present invention further provides use of compounds as defined herein for the manufacture of medicaments for the treatment of diseases where it is desirable to inhibit LRRK2. Such diseases include Parkinson's Disease.

### Pharmaceutical Compostions

For use according to the present invention, the compounds or physiologically acceptable salt, ester or other physiologically functional derivative thereof, described herein, may be presented as a pharmaceutical formulation, comprising the compounds or physiologically acceptable salt, ester or other physiologically functional derivative thereof, together with one or more pharmaceutically acceptable carriers therefore and optionally other therapeutic and/or prophylactic ingredients. The carrier(s) must be acceptable in the sense of being compatible with the other ingredients of the formulation and not deleterious to the recipient thereof. The pharmaceutical compositions may be for human or animal usage in human and veterinary medicine.

Examples of such suitable excipients for the various different forms of pharmaceutical compositions described herein may be found in the “Handbook of Pharmaceutical Excipients, 2^{nd} Edition, (1994), Edited by A Wade and PJ Weller.

Acceptable carriers or diluents for therapeutic use are well known in the pharmaceutical art, and are described, for example, in Remington's Pharmaceutical Sciences, Mack Publishing Co. (A. R. Gennaro edit. 1985).

Examples of suitable carriers include lactose, starch, glucose, methyl cellulose, magnesium stearate, mannitol, sorbitol and the like. Examples of suitable diluents include ethanol, glycerol and water.

The choice of pharmaceutical carrier, excipient or diluent can be selected with regard to the intended route of administration and standard pharmaceutical practice. The pharmaceutical compositions may comprise as, or in addition to, the carrier, excipient or diluent any suitable binder(s), lubricant(s), suspending agent(s), coating agent(s), solubilising agent(s), buffer(s), flavouring agent(s), surface active agent(s), thickener(s), preservative(s) (including anti-oxidants) and the like, and substances included for the purpose of rendering the formulation isotonic with the blood of the intended recipient.

Examples of suitable binders include starch, gelatin, natural sugars such as glucose, anhydrous lactose, free-flow lactose, beta-lactose, corn sweeteners, natural and synthetic gums, such as acacia, tragacanth or sodium alginate, carboxymethyl cellulose and polyethylene glycol.

Examples of suitable lubricants include sodium oleate, sodium stearate, magnesium stearate, sodium benzoate, sodium acetate, sodium chloride and the like.

Preservatives, stabilizers, dyes and even flavoring agents may be provided in the pharmaceutical composition. Examples of preservatives include sodium benzoate, sorbic acid and esters of p-hydroxybenzoic acid. Antioxidants and suspending agents may be also used.

Pharmaceutical formulations include those suitable for oral, topical (including dermal, buccal and sublingual), rectal or parenteral (including subcutaneous, intradermal, intramuscular and intravenous), nasal and pulmonary administration e.g., by inhalation. The formulation may, where appropriate, be conveniently presented in discrete dosage units and may be prepared by any of the methods well known in the art of pharmacy. All methods include the step of bringing into association an active compound with liquid carriers or finely divided solid carriers or both and then, if necessary, shaping the product into the desired formulation.

Pharmaceutical formulations suitable for oral administration wherein the carrier is a solid are most preferably presented as unit dose formulations such as boluses, capsules or tablets each containing a predetermined amount of active compound. A tablet may be made by compression or moulding, optionally with one or more accessory ingredients. Compressed tablets may be prepared by compressing in a suitable machine an active compound in a free-flowing form such as a powder or granules optionally mixed with a binder, lubricant, inert diluent, lubricating agent, surface-active agent or dispersing agent. Moulded tablets may be made by moulding an active compound with an inert liquid diluent. Tablets may be optionally coated and, if uncoated, may optionally be scored. Capsules may be prepared by filling an active compound, either alone or in admixture with one or more accessory ingredients, into the capsule shells and then sealing them in the usual manner. Cachets are analogous to capsules wherein an active compound together with any accessory ingredient(s) is sealed in a rice paper envelope. An active compound may also be formulated as dispersible granules, which may for example be suspended in water before administration, or sprinkled on food. The granules may be packaged, e.g., in a sachet. Formulations suitable for oral administration wherein the carrier is a liquid may be presented as a solution or a suspension in an aqueous or non-aqueous liquid, or as an oil-in-water liquid emulsion.

Formulations for oral administration include controlled release dosage forms, e.g., tablets wherein an active compound is formulated in an appropriate release—controlling matrix, or is coated with a suitable release—controlling film. Such formulations may be particularly convenient for prophylactic use.

Pharmaceutical formulations suitable for rectal administration wherein the carrier is a solid are most preferably presented as unit dose suppositories. Suitable carriers include cocoa butter and other materials commonly used in the art. The suppositories may be conveniently formed by admixture of an active compound with the softened or melted carrier(s) followed by chilling and shaping in moulds. Pharmaceutical formulations suitable for parenteral administration include sterile solutions or suspensions of an active compound in aqueous or oleaginous vehicles.

Injectable preparations may be adapted for bolus injection or continuous infusion. Such preparations are conveniently presented in unit dose or multi-dose containers which are sealed after introduction of the formulation until required for use. Alternatively, an active compound may be in powder form which is constituted with a suitable vehicle, such as sterile, pyrogen-free water, before use.

An active compound may also be formulated as long-acting depot preparations, which may be administered by intramuscular injection or by implantation, e.g., subcutaneously or intramuscularly. Depot preparations may include, for example, suitable polymeric or hydrophobic materials, or ion-exchange resins. Such long-acting formulations are particularly convenient for prophylactic use.

Formulations suitable for pulmonary administration via the buccal cavity are presented such that particles containing an active compound and desirably having a diameter in the range of 0.5 to 7 microns are delivered in the bronchial tree of the recipient.

As one possibility such formulations are in the form of finely comminuted powders which may conveniently be presented either in a pierceable capsule, suitably of, for example, gelatin, for use in an inhalation device, or alternatively as a self-propelling formulation comprising an active compound, a suitable liquid or gaseous propellant and optionally other ingredients such as a surfactant and/or a solid diluent. Suitable liquid propellants include propane and the chlorofluorocarbons, and suitable gaseous propellants include carbon dioxide. Self-propelling formulations may also be employed wherein an active compound is dispensed in the form of droplets of solution or suspension.

Such self-propelling formulations are analogous to those known in the art and may be prepared by established procedures. Suitably they are presented in a container provided with either a manually-operable or automatically functioning valve having the desired spray characteristics; advantageously the valve is of a metered type delivering a fixed volume, for example, 25 to 100 microlitres, upon each operation thereof.

As a further possibility an active compound may be in the form of a solution or suspension for use in an atomizer or nebuliser whereby an accelerated airstream or ultrasonic agitation is employed to produce a fine droplet mist for inhalation.

Formulations suitable for nasal administration include preparations generally similar to those described above for pulmonary administration. When dispensed such formulations should desirably have a particle diameter in the range 10 to 200 microns to enable retention in the nasal cavity; this may be achieved by, as appropriate, use of a powder of a suitable particle size or choice of an appropriate valve. Other suitable formulations include coarse powders having a particle diameter in the range 20 to 500 microns, for administration by rapid inhalation through the nasal passage from a container held close up to the nose, and nasal drops comprising 0.2 to 5% w/v of an active compound in aqueous or oily solution or suspension.

Pharmaceutically acceptable carriers are well known to those skilled in the art and include, but are not limited to, 0.1 M and preferably 0.05 M phosphate buffer or 0.8% saline. Additionally, such pharmaceutically acceptable carriers may be aqueous or non-aqueous solutions, suspensions, and emulsions. Examples of non-aqueous solvents are propylene glycol, polyethylene glycol, vegetable oils such as olive oil, and injectable organic esters such as ethyl oleate. Aqueous carriers include water, alcoholic/aqueous solutions, emulsions or suspensions, including saline and buffered media. Parenteral vehicles include sodium chloride solution, Ringer's dextrose, dextrose and sodium chloride, lactated Ringer's or fixed oils. Preservatives and other additives may also be present, such as, for example, antimicrobials, antioxidants, chelating agents, inert gases and the like.

Formulations suitable for topical formulation may be provided for example as gels, creams or ointments. Such preparations may be applied e.g. to a wound or ulcer either directly spread upon the surface of the wound or ulcer or carried on a suitable support such as a bandage, gauze, mesh or the like which may be applied to and over the area to be treated.

Liquid or powder formulations may also be provided which can be sprayed or sprinkled directly onto the site to be treated, e.g. a wound or ulcer. Alternatively, a carrier such as a bandage, gauze, mesh or the like can be sprayed or sprinkle with the formulation and then applied to the site to be treated.

According to a further aspect of the invention, there is provided a process for the preparation of a pharmaceutical or veterinary composition as described above, the process comprising bringing the active compound(s) into association with the carrier, for example by admixture.

In general, the formulations are prepared by uniformly and intimately bringing into association the active agent with liquid carriers or finely divided solid carriers or both, and then if necessary shaping the product. The invention extends to methods for preparing a pharmaceutical composition comprising bringing a compound of general formula (I) in conjunction or association with a pharmaceutically or veterinarily acceptable carrier or vehicle.

### Salts/Esters

The compounds of the invention can be present as salts or esters, in particular pharmaceutically and veterinarily acceptable salts or esters.

Pharmaceutically acceptable salts of the compounds of the invention include suitable acid addition or base salts thereof. A review of suitable pharmaceutical salts may be found in Berge et al, J Pharm Sci, 66, 1-19 (1977). Salts are formed, for example with strong inorganic acids such as mineral acids, e.g. hydrohalic acids such as hydrochloride, hydrobromide and hydroiodide, sulphuric acid, phosphoric acid sulphate, bisulphate, hemisulphate, thiocyanate, persulphate and sulphonic acids; with strong organic carboxylic acids, such as alkanecarboxylic acids of 1 to 4 carbon atoms which are unsubstituted or substituted (e.g., by halogen), such as acetic acid; with saturated or unsaturated dicarboxylic acids, for example oxalic, malonic, succinic, maleic, fumaric, phthalic or tetraphthalic; with hydroxycarboxylic acids, for example ascorbic, glycolic, lactic, malic, tartaric or citric acid; with aminoacids, for example aspartic or glutamic acid; with benzoic acid; or with organic sulfonic acids, such as (C₁-C₄)-alkyl- or aryl-sulfonic acids which are unsubstituted or substituted (for example, by a halogen) such as methane- or p-toluene sulfonic acid. Salts which are not pharmaceutically or veterinarily acceptable may still be valuable as intermediates.

Preferred salts include, for example, acetate, trifluoroacetate, lactate, gluconate, citrate, tartrate, maleate, malate, pantothenate, adipate, alginate, aspartate, benzoate, butyrate, digluconate, cyclopentanate, glucoheptanate, glycerophosphate, oxalate, heptanoate, hexanoate, fumarate, nicotinate, palmoate, pectinate, 3-phenylpropionate, picrate, pivalate, proprionate, tartrate, lactobionate, pivolate, camphorate, undecanoate and succinate, organic sulphonic acids such as methanesulphonate, ethanesulphonate, 2-hydroxyethane sulphonate, camphorsulphonate, 2-naphthalenesulphonate, benzenesulphonate, p-chlorobenzenesulphonate and p-toluenesulphonate; and inorganic acids such as hydrochloride, hydrobromide, hydroiodide, sulphate, bisulphate, hemisulphate, thiocyanate, persulphate, phosphoric and sulphonic acids.

Esters are formed either using organic acids or alcohols/hydroxides, depending on the functional group being esterified. Organic acids include carboxylic acids, such as alkanecarboxylic acids of 1 to 12 carbon atoms which are unsubstituted or substituted (e.g., by halogen), such as acetic acid; with saturated or unsaturated dicarboxylic acid, for example oxalic, malonic, succinic, maleic, fumaric, phthalic or tetraphthalic; with hydroxycarboxylic acids, for example ascorbic, glycolic, lactic, malic, tartaric or citric acid; with aminoacids, for example aspartic or glutamic acid; with benzoic acid; or with organic sulfonic acids, such as (C₁-C₄)-alkyl- or aryl-sulfonic acids which are unsubstituted or substituted (for example, by a halogen) such as methane- or p-toluene sulfonic acid. Suitable hydroxides include inorganic hydroxides, such as sodium hydroxide, potassium hydroxide, calcium hydroxide, aluminium hydroxide. Alcohols include alkanealcohols of 1-12 carbon atoms which may be unsubstituted or substituted, e.g. by a halogen).

### Enantiomers/Tautomers

In all aspects of the present invention previously discussed, the invention includes, where appropriate all enantiomers, diastereoisomers and tautomers of the compounds of the invention. The person skilled in the art will recognise compounds that possess optical properties (one or more chiral carbon atoms) or tautomeric characteristics. The corresponding enantiomers and/or tautomers may be isolated/prepared by methods known in the art.

Enantiomers are characterised by the absolute configuration of their chiral centres and described by the R- and S-sequencing rules of Cahn, Ingold and Prelog. Such conventions are well known in the art (e.g. see ‘Advanced Organic Chemistry’, 3^{rd} edition, ed. March, J., John Wiley and Sons, New York, 1985).

Compounds of the invention containing a chiral centre may be used as a racemic mixture, an enantiomerically enriched mixture, or the racemic mixture may be separated using well-known techniques and an individual enantiomer may be used alone.

### Stereo and Geometric Isomers

Some of the compounds of the invention may exist as stereoisomers and/or geometric isomers—e.g. they may possess one or more asymmetric and/or geometric centres and so may exist in two or more stereoisomeric and/or geometric forms. The present invention contemplates the use of all the individual stereoisomers and geometric isomers of those inhibitor agents, and mixtures thereof. The terms used in the claims encompass these forms, provided said forms retain the appropriate functional activity (though not necessarily to the same degree).

The present invention also includes all suitable isotopic variations of the agent or a pharmaceutically acceptable salt thereof. An isotopic variation of an agent of the present invention or a pharmaceutically acceptable salt thereof is defined as one in which at least one atom is replaced by an atom having the same atomic number but an atomic mass different from the atomic mass usually found in nature. Examples of isotopes that can be incorporated into the agent and pharmaceutically acceptable salts thereof include isotopes of hydrogen, carbon, nitrogen, oxygen, phosphorus, sulphur, fluorine and chlorine such as ²H, ³H, ¹³C, ¹⁴C, ¹⁵N, ¹⁷O, ¹⁸O, ³¹P, ³²P, ³⁵S, ¹⁸F and ³⁵Cl, respectively. Certain isotopic variations of the agent and pharmaceutically acceptable salts thereof, for example, those in which a radioactive isotope such as ³H or ¹⁴C is incorporated, are useful in drug and/or substrate tissue distribution studies. Tritiated, i.e., ³H, and carbon-14, i.e., ¹⁴C, isotopes are particularly preferred for their ease of preparation and detectability. Further, substitution with isotopes such as deuterium, i.e., ²H, may afford certain therapeutic advantages resulting from greater metabolic stability, for example, increased in vivo half-life or reduced dosage requirements and hence may be preferred in some circumstances. For example, the invention includes compounds of general formula (I) where any hydrogen atom has been replaced by a deuterium atom. Isotopic variations of the agent of the present invention and pharmaceutically acceptable salts thereof of this invention can generally be prepared by conventional procedures using appropriate isotopic variations of suitable reagents.

### Prodrugs

The invention further includes the compounds of the present invention in prodrug form, i.e. covalently bonded compounds which release the active parent drug according to general formula (I) in vivo. Such prodrugs are generally compounds of the invention wherein one or more appropriate groups have been modified such that the modification may be reversed upon administration to a human or mammalian subject. Reversion is usually performed by an enzyme naturally present in such subject, though it is possible for a second agent to be administered together with such a prodrug in order to perform the reversion in vivo. Examples of such modifications include ester (for example, any of those described above), wherein the reversion may be carried out be an esterase etc. Other such systems will be well known to those skilled in the art.

### Solvates

The present invention also includes solvate forms of the compounds of the present invention. The terms used in the claims encompass these forms.

### Polymorphs

The invention further relates to the compounds of the present invention in their various crystalline forms, polymorphic forms and (an)hydrous forms. It is well established within the pharmaceutical industry that chemical compounds may be isolated in any of such forms by slightly varying the method of purification and or isolation form the solvents used in the synthetic preparation of such compounds.

### Administration

The pharmaceutical compositions of the present invention may be adapted for rectal, nasal, intrabronchial, topical (including buccal and sublingual), vaginal or parenteral (including subcutaneous, intramuscular, intravenous, intraarterial and intradermal), intraperitoneal or intrathecal administration. Preferably the formulation is an orally administered formulation. The formulations may conveniently be presented in unit dosage form, i.e., in the form of discrete portions containing a unit dose, or a multiple or sub-unit of a unit dose. By way of example, the formulations may be in the form of tablets and sustained release capsules, and may be prepared by any method well known in the art of pharmacy.

Formulations for oral administration in the present invention may be presented as: discrete units such as capsules, gellules, drops, cachets, pills or tablets each containing a predetermined amount of the active agent; as a powder or granules; as a solution, emulsion or a suspension of the active agent in an aqueous liquid or a non-aqueous liquid; or as an oil-in-water liquid emulsion or a water-in-oil liquid emulsion; or as a bolus etc. Preferably, these compositions contain from 1 to 250 mg and more preferably from 10-100 mg, of active ingredient per dose.

For compositions for oral administration (e.g. tablets and capsules), the term “acceptable carrier” includes vehicles such as common excipients e.g. binding agents, for example syrup, acacia, gelatin, sorbitol, tragacanth, polyvinylpyrrolidone (Povidone), methylcellulose, ethylcellulose, sodium carboxymethylcellulose, hydroxypropyl-methylcellulose, sucrose and starch; fillers and carriers, for example corn starch, gelatin, lactose, sucrose, microcrystalline cellulose, kaolin, mannitol, dicalcium phosphate, sodium chloride and alginic acid; and lubricants such as magnesium stearate, sodium stearate and other metallic stearates, glycerol stearate stearic acid, silicone fluid, talc waxes, oils and colloidal silica. Flavouring agents such as peppermint, oil of wintergreen, cherry flavouring and the like can also be used. It may be desirable to add a colouring agent to make the dosage form readily identifiable. Tablets may also be coated by methods well known in the art.

A tablet may be made by compression or moulding, optionally with one or more accessory ingredients. Compressed tablets may be prepared by compressing in a suitable machine the active agent in a free flowing form such as a powder or granules, optionally mixed with a binder, lubricant, inert diluent, preservative, surface-active or dispersing agent. Moulded tablets may be made by moulding in a suitable machine a mixture of the powdered compound moistened with an inert liquid diluent. The tablets may be optionally be coated or scored and may be formulated so as to provide slow or controlled release of the active agent.

Other formulations suitable for oral administration include lozenges comprising the active agent in a flavoured base, usually sucrose and acacia or tragacanth; pastilles comprising the active agent in an inert base such as gelatin and glycerin, or sucrose and acacia; and mouthwashes comprising the active agent in a suitable liquid carrier.

Other forms of administration comprise solutions or emulsions which may be injected intravenously, intraarterially, intrathecally, subcutaneously, intradermally, intraperitoneally or intramuscularly, and which are prepared from sterile or sterilisable solutions. Injectable forms typically contain between 10-1000 mg, preferably between 10-250 mg, of active ingredient per dose.

The pharmaceutical compositions of the present invention may also be in form of suppositories, pessaries, suspensions, emulsions, lotions, ointments, creams, gels, sprays, solutions or dusting powders.

An alternative means of transdermal administration is by use of a skin patch. For example, the active ingredient can be incorporated into a cream consisting of an aqueous emulsion of polyethylene glycols or liquid paraffin. The active ingredient can also be incorporated, at a concentration of between 1 and 10% by weight, into an ointment consisting of a white wax or white soft paraffin base together with such stabilisers and preservatives as may be required.

### Dosage

A person of ordinary skill in the art can easily determine an appropriate dose of one of the instant compositions to administer to a subject without undue experimentation. Typically, a physician will determine the actual dosage which will be most suitable for an individual patient and it will depend on a variety of factors including the activity of the specific compound employed, the metabolic stability and length of action of that compound, the age, body weight, general health, sex, diet, mode and time of administration, rate of excretion, drug combination, the severity of the particular condition, and the individual undergoing therapy. The dosages disclosed herein are exemplary of the average case. There can of course be individual instances where higher or lower dosage ranges are merited, and such are within the scope of this invention.

In accordance with this invention, an effective amount of a compound of general formula (I) may be administered to inhibit the kinase implicated with a particular condition or disease. Of course, this dosage amount will further be modified according to the type of administration of the compound. For example, to achieve an “effective amount” for acute therapy, parenteral administration of a compound of general formula (I) is preferred. An intravenous infusion of the compound in 5% dextrose in water or normal saline, or a similar formulation with suitable excipients, is most effective, although an intramuscular bolus injection is also useful. Typically, the parenteral dose will be about 0.01 to about 100 mg/kg; preferably between 0.1 and 20 mg/kg, in a manner to maintain the concentration of drug in the plasma at a concentration effective to inhibit a kinase. The compounds may be administered one to four times daily at a level to achieve a total daily dose of about 0.4 to about 400 mg/kg/day. The precise amount of an inventive compound which is therapeutically effective, and the route by which such compound is best administered, is readily determined by one of ordinary skill in the art by comparing the blood level of the agent to the concentration required to have a therapeutic effect.

The compounds of this invention may also be administered orally to the patient, in a manner such that the concentration of drug is sufficient to achieve one or more of the therapeutic indications disclosed herein. Typically, a pharmaceutical composition containing the compound is administered at an oral dose of between about 0.1 to about 50 mg/kg in a manner consistent with the condition of the patient. Preferably the oral dose would be about 0.5 to about 20 mg/kg.

No unacceptable toxicological effects are expected when compounds of the present invention are administered in accordance with the present invention. The compounds of this invention, which may have good bioavailability, may be tested in one of several biological assays to determine the concentration of a compound which is required to have a given pharmacological effect.

### Combinations

In a particularly preferred embodiment, the one or more compounds of the invention are administered in combination with one or more other active agents, for example, existing drugs available on the market. In such cases, the compounds of the invention may be administered consecutively, simultaneously or sequentially with the one or more other active agents.

Drugs in general are more effective when used in combination. In particular, combination therapy is desirable in order to avoid an overlap of major toxicities, mechanism of action and resistance mechanism(s). Furthermore, it is also desirable to administer most drugs at their maximum tolerated doses with minimum time intervals between such doses. The major advantages of combining chemotherapeutic drugs are that it may promote additive or possible synergistic effects through biochemical interactions and also may decrease the emergence of resistance.

Beneficial combinations may be suggested by studying the inhibitory activity of the test compounds with agents known or suspected of being valuable in the treatment of a particular disorder. This procedure can also be used to determine the order of administration of the agents, i.e. before, simultaneously, or after delivery. Such scheduling may be a feature of all the active agents identified herein.

### Assay

A further aspect of the invention relates to the use of a compound as described above in an assay for identifying further candidate compounds capable of inhibiting one or more kinases, more preferably LRRK, even more preferably, LRRK2.

Preferably, the assay is a competitive binding assay.

More preferably, the competitive binding assay comprises contacting a compound of the invention with a kinase, preferably LRRK, more preferably LRRK2, and a candidate compound and detecting any change in the interaction between the compound according to the invention and the kinase.

Preferably, the candidate compound is generated by conventional SAR modification of a compound of the invention.

As used herein, the term “conventional SAR modification” refers to standard methods known in the art for varying a given compound by way of chemical derivatisation.

Thus, in one aspect, the identified compound may act as a model (for example, a template) for the development of other compounds. The compounds employed in such a test may be free in solution, affixed to a solid support, borne on a cell surface, or located intracellularly. The abolition of activity or the formation of binding complexes between the compound and the agent being tested may be measured.

The assay of the present invention may be a screen, whereby a number of agents are tested. In one aspect, the assay method of the present invention is a high through-put screen.

This invention also contemplates the use of competitive drug screening assays in which neutralising antibodies capable of binding a compound specifically compete with a test compound for binding to a compound.

Another technique for screening provides for high throughput screening (HTS) of agents having suitable binding affinity to the substances and is based upon the method described in detail in WO 84/03564.

It is expected that the assay methods of the present invention will be suitable for both small and large-scale screening of test compounds as well as in quantitative assays.

Preferably, the competitive binding assay comprises contacting a compound of the invention with a kinase in the presence of a known substrate of said kinase and detecting any change in the interaction between said kinase and said known substrate.

A further aspect of the invention provides a method of detecting the binding of a ligand to a kinase, said method comprising the steps of:
(i) contacting a ligand with a kinase in the presence of a known substrate of said kinase;
(ii) detecting any change in the interaction between said kinase and said known substrate;
   and wherein said ligand is a compound of the invention.

One aspect of the invention relates to a process comprising the steps of:
(a) performing an assay method described hereinabove;
(b) identifying one or more ligands capable of binding to a ligand binding domain; and
(c) preparing a quantity of said one or more ligands.

Another aspect of the invention provides a process comprising the steps of:
(a) performing an assay method described hereinabove;
(b) identifying one or more ligands capable of binding to a ligand binding domain; and
(c) preparing a pharmaceutical composition comprising said one or more ligands.

Another aspect of the invention provides a process comprising the steps of:
(a) performing an assay method described hereinabove;
(b) identifying one or more ligands capable of binding to a ligand binding domain;
(c) modifying said one or more ligands capable of binding to a ligand binding domain;
(d) performing the assay method described hereinabove;
(e) optionally preparing a pharmaceutical composition comprising said one or more ligands.

The invention also relates to a ligand identified by the method described hereinabove.

Yet another aspect of the invention relates to a pharmaceutical composition comprising a ligand identified by the method described hereinabove.

Another aspect of the invention relates to the use of a ligand identified by the method described hereinabove in the preparation of a pharmaceutical composition for use in the treatment of one or more disorders described above.

The above methods may be used to screen for a ligand useful as an inhibitor of one or more kinases.

The compounds of the invention are useful both as laboratory tools and as therapeutic agents. In the laboratory certain compounds of the invention are useful in establishing whether a known or newly discovered kinase contributes a critical or at least significant biochemical function during the establishment or progression of a disease state, a process commonly referred to as ‘target validation’.

### Synthesis

Another aspect of the invention relates to a process for preparing compounds of formula Ia and formula Ib.

More specifically, the invention provides a process for preparing a compound of formula Ia′, where Q′ is halogen or C₁₋₆-alkyl, and R¹ and R² are as defined above, said process comprising converting a compound of formula IIa′ into a compound of formula Ia′:

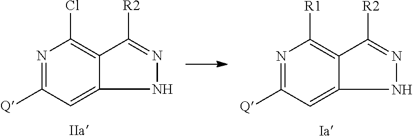

In one preferred embodiment of the invention, the process further comprises the step of preparing said compound of formula IIa′ by treating a compound of formula IIIa′ with hydrazine monohydrate:

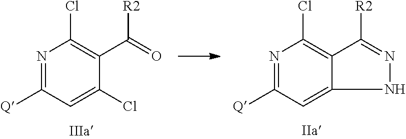

In one preferred embodiment of the invention, the process further comprises the step of preparing said compound of formula IIIa′ by treating a compound of formula IVa′ with an oxidizing agent:

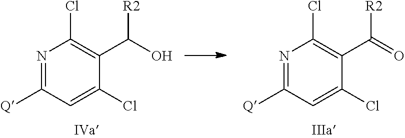

In one preferred embodiment of the invention, the process further comprises the step of preparing said compound of formula IVa′ by treating a compound of formula Va′ with R²—Mg—Cl:

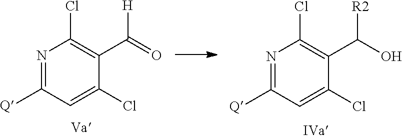

In one preferred embodiment of the invention, R¹ is —NHR³, and the process comprises reacting a compound of formula IIa′ with an amine of formula NH₂R³.

In another preferred embodiment of the invention, R¹ is an NH-containing C₄₋₇-heterocycloalkyl or an NH-containing fused aryl-C₄₋₇-heterocycloalkyl, and the process comprises reacting a compound of formula IIa′ with the NH-group of said C₄₋₇-heterocycloalkyl or fused aryl-C₄₋₇-heterocycloalkyl.

In another preferred embodiment of the invention, R¹ is selected from aryl, heteroaryl, C₄₋₇-heterocycloalkyl, fused aryl-C₄₋₇-heterocycloalkyl, —C₃₋₇ cycloalkyl and —C″ alkyl, and said process comprises reacting a compound of formula IIa′ with X—R¹, where X is a 4,4,5,5-tetramethyl-1,3,2-dioxaborolan-2-yl group, in the presence of a coupling agent.

Preferably, the coupling agent is palladium diphenylphosphinoferrocene dichloride.

Another aspect of the invention relates to a process for preparing a compound of formula Ia″, wherein Q″ is C₃₋₇-cycloalkyl, heterocycloalkyl, aryl or heteroaryl, each of which is optionally substituted with one or more substituents A, and R¹ and R² are as defined above, said process comprising converting a compound of formula VIa″ into a compound of formula Ia″:

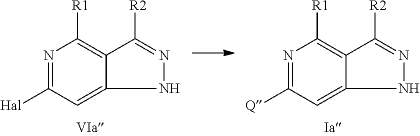

In one preferred embodiment, the process comprises reacting a compound of formula VIa″ with the NH-group of a C₄₋₇-heterocycloalkyl in the presence of a coupling agent.

In another preferred embodiment, the process comprises reacting a compound of formula VIa″ with a compound Q″—Y, where Q″ is C₃₋₇-cycloalkyl, heterocycloalkyl, aryl or heteroaryl and Y is a boronic acid or boronic acid ester moiety, in the presence of a coupling agent.

Another aspect of the invention relates to a process for preparing a compound of formula Ib as defined above, said process comprising converting a compound of formula IIb into a compound of formula Ib,

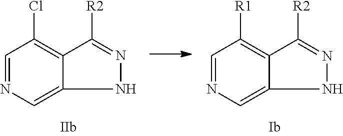

Preferably, said compound of formula IIb is prepared from a compound of formula IIIb,

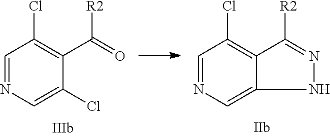

by treating said compound of formula IIIb with a hydrazine, preferably hydrazine monohydrate.

In one preferred embodiment of the invention, the process further comprises the step of preparing said compound of formula IIIb by treating a compound of formula IVb with an oxidizing agent:

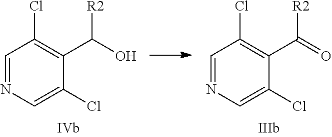

In one preferred embodiment of the invention, the process further comprises the step of preparing said compound of formula IVb by treating a compound of formula Vb with R²—Mg—Cl.

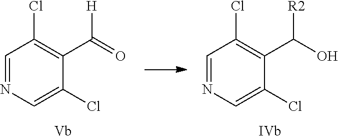

In one preferred embodiment, R¹ is aryl or heteroaryl, and the process comprises reacting said compound of formula IIb with a compound R¹—Y, where Y is a boronic acid or boronic acid ester moiety, in the presence of a coupling agent.

In another preferred embodiment, R¹ is —NHR³, and the process comprises reacting said compound of formula IIb with an amine of formula NH₂R³.

Another aspect of the invention relates to a process for preparing a compound of formula Ib as defined above, wherein R¹ is OR³, said process comprising converting a compound of formula IIIb into a compound of formula Ib,

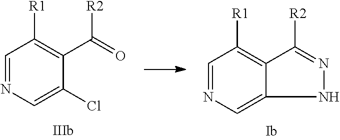

Preferably, for this embodiment the process comprises treating said compound of formula IIIb with a hydrazine, preferably hydrazine monohydrate, and subjecting the reaction mixture to microwave radiation.

Further aspects relating to processes for preparing compounds of formula Ia and formula Ib are described in the accompanying Examples section.

The invention is further described by way of the following non-limiting examples, and with reference to the following figures, wherein:

FIG. 1 shows the domain structure of LRRK1 and local mutations that have been linked to Parkinson's disease.

### EXAMPLES

### Materials and Methods

### Source and Purification of Kinases

All LRRK2 protein kinases were of human origin and were sourced from Invitrogen Corporation (Carlsbad, Calif. 92008 USA) unless otherwise indicated. The active mutant used was recombinant human, catalytic domain (amino acids 970-2527) containing a G2019S mutation, GST-tagged, expressed in insect cells (Invitrogen Cat#PV4881). The wild type used was recombinant human, catalytic domain (amino acids 970-2527) GST—tagged, expressed in insect cells (Invitrogen Cat#PV4873). The kinase dead mutant used was recombinant human, catalytic domain (amino acids 970-2527) containing a D1994A mutation, GST-tagged, expressed in insect cells (Invitrogen Cat#PM4041AE). No special measures were taken to activate any of the kinases.

### Protein Kinase Assays

All assays were carried out at room temperature (−21° C.) and were linear with respect to time and enzyme concentration under the conditions used. Assays were performed for 180 min in a 96 well format. LRRK2 was present at a concentration of approximately 5 nM.

The enzyme was diluted and assayed in 50 mM Tris-HCl pH7.5, 0.1 mM EGTA, 1 mM DTT and 10 mM MgCl₂. The concentration of magnesium chloride in the assay was 10 mM. The [γ-33P] ATP (0.4 μCi/well) was used at 134 uM for G2019S mutant and at 57 μM for the wild type kinase in order to be at Km. The peptide substrate in the assay was RLGWWRFYTLRRARQGNTKQR at 100 μM.

The assays were initiated with Mg/ATP and stopped by the addition of 25 μl/well 50% orthophosphoric acid. Reactions were harvested onto Whatman P81 Unifilter Plates (Fisher Scientific. Loughborough, LE115RG, UK. Cat# FDU-105-020U) using a Tomtec harvester (Tomtec Hamden, Conn. 06514. USA). Plates were counted using a Perkin Elmer Top Count NX7. (Perkin Elmer, Shelton CT 06484-4794 USA)

IC50 values of inhibitors were determined after carrying out assays at 10 different concentrations of each compound in duplicate.

### General Procedures for Synthesis of Compounds

### Chromatography

Preparative high pressure liquid chromatography was carried out using apparatus made by Agilent. The apparatus is constructed such that the chromatography is monitored by a multi-wavelength UV detector (G1365B manufactured by Agilent) and an MM-ES+APCI mass spectrometer (G-1956A, manufactured by Agilent) connected in series, and if the appropriate criteria are met the sample is collected by an automated fraction collector (G1364B manufactured by Agilent). Collection can be triggered by any combination of UV or mass spectrometry or can be based on time. Typical conditions for the separation process are as follows: The gradient is run over a 10 minute period (gradient at start: 10% methanol and 90% water, gradient at finish: 100% methanol and 0% water; as buffer: either 0.1% trifluoroacetic acid is added to the water (low pH buffer), or ammonium bicarbonate (10 mmol/l) and 35% ammonium hydroxide (1.6 ml/l) is added to the water (high pH buffer). It will be appreciated by those skilled in the art that it may be necessary or desirable to modify the conditions for each specific compound, for example by changing the solvent composition at the start or at the end, modifying the solvents or buffers, changing the run time, changing the flow rate and/or the chromatography column.

Flash chromatography refers to silica gel chromatography and carried out using an SP4 or an Isolara 4 MPLC system (manufactured by Biotage); pre-packed silica gel cartridges (supplied by Biotage); or using conventional glass column chromatography.

### Analytical Methods

¹H Nuclear magnetic resonance (NMR) spectroscopy was carried out using an ECX400 spectrometer (manufactured by JEOL) in the stated solvent at around room temperature unless otherwise stated. In all cases, NMR data were consistent with the proposed structures. Characteristic chemical shifts (δ) are given in parts-per-million using conventional abbreviations for designation of major peaks: e.g. s, singlet; d, doublet; t, triplet; q, quartet; dd, doublet of doublets; br, broad. Mass spectra were recorded using a MM-ES+APCI mass spectrometer (G-1956A, manufactured by Agilent). Where thin layer chromatography (TLC) has been used it refers to silica gel TLC using silica gel MK6F 60A plates, R_{f} is the distance travelled by the compound divided by the distance travelled by the solvent on a TLC plate.

### Compound Preparation

Where the preparation of starting materials is not described, these are commercially available, known in the literature, or readily obtainable by those skilled in the art using standard procedures. Where it is stated that compounds were prepared analogously to earlier examples or intermediates, it will be appreciated by the skilled person that the reaction time, number of equivalents of reagents and temperature can be modified for each specific reaction and that it may be necessary or desirable to employ different work-up or purification techniques. Where reactions are carried out using microwave irradiation, the microwave used is an Initiator 60 supplied by Biotage. The actual power supplied varies during the course of the reaction in order to maintain a constant temperature.

### ABBREVIATIONS

DCM=Dichloromethane
DMF=N,N-Dimethylformamide
THF=Tetrahydrofuran
MeOH=Methanol
TFA=Trifluoroacetic acid
Xantphos=4,5-Bis(diphenylphosphino)-9,9-dimethylxanthene
HATU=N,N,N′,N′-Tetramethyl-O-(7-azabenzotriazol-1-yl)uronium-hexafluorophospate
EDCI=1,3-Propanediamine, N3-(ethylcarbonimidoyl)-N1,N1-dimethyl-, hydrochloride
DCC=1,3-Dicyclohexylcarbodiimide
Pd2(dba)₃=tris(dibenzylideneacetone)dipalladium(0)
TEA=Triethylamine
rm=Reaction mixture
rt=Room temperature
AcOH=Acetic acid
IPA=Isopropanol
DIPEA=N,N-diisopropylethylamine
TBSMSCl=Tertiarybutyldimethylsilyl chloride
MeCN=Acetonitrile
NH₃=Ammonia
EtOH=Ethanol
EtOAc=Ethyl Acetate
LCMS=Mass spectrometry directed high pressure liquid chromatography
UV=Ultraviolet
SCX=Strong cation exchange
TPAP=Tetrapropylammonium perruthenate
DMSO=Dimethylsulphoxide
BINAP=2,2′-bis(diphenylphosphino)-1,1′-binaphthyl
TPAP=Tetrapropylammonium perruthenate
DIAD=Diisopropyl azodicarboxylate
NMO=N-Methylmorpholine N-oxide

### Intermediate 1

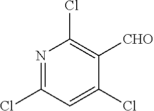 [SMILES: O=Cc1c(Cl)cc(Cl)nc1Cl]

A solution of 2,4,6-trichloropyridine (9.85 g, 53.8 mmol) in THF (100 ml) was added dropwise to a solution of n-butyllithium, 1.6M in hexane (33.6 ml, 53.8 mmol) in THF (40 ml) whilst maintaining the temperature below −73° C. After the addition was complete, the mixture was stirred at −78° C. for 1 hour. A solution of 1-formylpiperidine (6.0 ml, 53.8 mmol) in THF (10 ml) was added dropwise and the mixture was stirred at −78° C. for 1 hour. The reaction mixture was quenched with sat. NH₄Cl(aq) at −78° C. and allowed to warm to rt. The mixture was extracted with ether and was washed successively with 1M HCl(aq), water, sat. NH₄HCO₃(aq), water and brine. The organic phase was dried and concentrated. The residue was purified by flash column chromatography on silica gel eluting with 10:1 petrol-ethyl acetate to provide a yellow solid (5.18 g, 45%). ¹H NMR (400 MHz, DMSO-d₆) δ ppm 8.09 (s, 1H), 10.27 (s, 1H).

### Intermediate 2

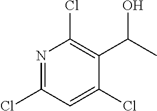

A solution of methylmagnesium chloride, 3M in THF (9.0 ml, 26.9 mmol) in THF (10 ml) was added dropwise to a stirred solution of Intermediate 1 (5.18 g, 24.4 mmol) in THF (110 ml) at −78° C. After the addition the reaction mixture was stirred at −78° C. for 30 minutes and then allowed to warm to rt. The mixture was quenched with sat. NH₄Cl(aq) and extracted with ethyl acetate. The organic phase was washed with brine, dried and concentrated to give a yellow oil (5.57 g, 100%). ¹H NMR (400 MHz, DMSO-d₆) δ ppm 1.46 (d, J=6.9 Hz, 3H), 5.36 (m, 1H), 5.57 (d, J=4.1 Hz, 1H), 7.84 (s, 1H).

### Intermediate 3

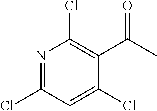

NMO (4.3 g, 36.6 mmol) and 4 Å molecular sieves (6.4 g) were added to a stirred solution of Intermediate 2 (5.57 g, 24.4 mmol) in DCM (100 ml). After 15 minutes TPAP (288 mg, 0.82 mmol) was added and the reaction mixture was stirred at rt for 1 hour. The mixture was filtered through Celite and the filtrate was concentrated, and the residue was purified by flash column chromatography on silica gel eluting with 7:1 petrol-ethyl acetate to provide a yellow oil (4.6 g, 83%). ¹H NMR (400 MHz, DMSO-d₆) δ ppm 2.59 (s, 3H), 8.08 (s, 1H).

### Intermediate 4

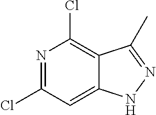

A mixture of

Intermediate 3 (3.55 g, 15.8 mmol) and 35% aqueous hydrazine (35 ml) in ethanol (35 ml) was stirred at it for 3.5 hours. The reaction mixture was added to ice and extracted with ethyl acetate. The organic phase was washed with brine, dried and concentrated. The residue was purified by flash column chromatography on silica gel eluting with 3:1 petrol-ethyl acetate to give an off-white coloured solid (1.71 g, 54%). ¹H NMR (400 MHz, DMSO-d₆) δ ppm 2.64 (s, 3H), 7.63 (s, 1H).

### Intermediate 5

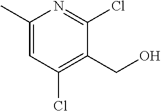

A solution of 2,4-dichloro-6-methylnicotinate (15 g, 64.1 mmol) in THF (80 ml) was added dropwise to lithium aluminium hydride, 2M in THF (160 ml, 320 mmol) at −78° C. A precipitate was observed before the ester addition and more precipitate formed during the addition so more THF (100 ml) was added in order to mobilize the mixture. The reaction mixture was stirred at −78° C. for 5 hours and then at −30° C. for 1 hour. Water (10.3 ml) was then added very slowly at −30° C. followed by the very slow addition of 15% aqueous sodium hydroxide solution (10.3 ml) and finally by the addition of more water (32 ml). The mixture was allowed to warm to rt and stirred overnight. The mixture was filtered through Celite and the filtrate concentrated. The residue was purified by flash column chromatography on silica gel eluting with 4:1 petrol-ethyl acetate to afford an off-white solid (9.35 g, 76%). ¹H-NMR (400 MHz, DMSO-d₆) δ ppm 2.43 (s, 3H), 4.63 (d, J=5.5 Hz, 2H), 5.30-5.34 (m, 1H), 7.49 (s, 1H). m/z (ES+APCI)⁺: 192/194/196 [M+H]⁺.

### Intermediate 6

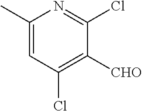

DMSO (20 ml, 282 mmol) was added to a stirred solution of oxalyl chloride (12.1 ml, 140 mmol) in DCM (140 ml) at −78° C. After the addition, Intermediate 5 (9.32 g, 49 mmol) in DCM (35 ml) was then added followed by the addition of Et₃N (79 ml, 568 mmol) whilst maintaining the temperature below −70° C. The reaction mixture was allowed to warm to rt and stirred for 1 hour. The reaction mixture was washed with NaHCO₃(aq) solution and the organic phase was dried and concentrated. The residue was purified by flash column chromatography on silica gel eluting with 10:1 petrol-ethyl acetate to give an off-white solid (7.88 g, 85%). ¹H NMR (400 MHz, chloroform-d) δ ppm 2.60 (s, 3H), 7.27 (s, 1H), 10.46 (s, 1H).

### Intermediate 7

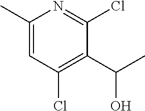

Methyl magnesium chloride, 3M in THF (5.8 ml, 17.4 mmol) was added to a solution of Intermediate 6 (3.0 g, 15.8 mmol) in THF (60 ml) at −78° C. The mixture was stirred at −78° C. for 45 minutes and then allowed to warm to rt. The mixture was quenched with sat. NH₄Cl(aq), diluted with water and extracted with ethyl acetate. The organic phase was washed with brine, dried and concentrated to give a yellow oil (3.23 g, 99%). ¹H NMR (400 MHz, DMSO-d₆) δ ppm 1.45 (d, J=6.4 Hz, 3H), 2.40 (s, 3H), 5.31-5.40 (m, 1H), 5.44 (d, J=4.1 Hz, 1H), 7.43 (s, 1H). m/z (ES+APCI)⁺: 206 [m+H]⁺.

### Intermediate 8

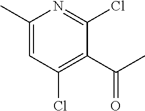

Freshly activated 4 Å molecular sieves (5.75 g) and NMO (4.19 g, 35.8 mmol) were added to a solution of Intermediate 7 (2.95 g, 14.3 mmol) in DCM (100 ml). The mixture was stirred at it for 15 minutes followed by the addition of TPAP (256 mg, 0.729 mmol). The reaction mixture was stirred at it for 45 minutes and then filtered through Celite, and the filtrate was concentrated to dryness. The residue was purified by flash column chromatography on silica gel in 6:1 petrol-ethyl acetate to give a yellow oil (2.31 g, 79%). ¹H NMR (400 MHz, chloroform-d) δ ppm 2.55 (s, 3H), 2.60 (s, 3H), 7.19 (s, 1H).

### Intermediate 9

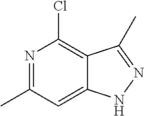

A mixture of intermediate 7 (232 mg, 1.14 mmol) in 65% aqueous hydrazine (2 ml) was stirred at it overnight. The mixture was diluted with DCM and water and MeOH was added to dissolve solid that was present. The organic phase was separated and the aqueous re-extracted with DCM. The combined organic extracts were dried and concentrated to give a white solid. The solid was recrystallised from ethyl acetate to give an off-white crystalline solid (50 mg, 24%). ¹H NMR (400 MHz, DMSO-d₆) δ ppm 2.49 (s, 3H), 2.61 (s, 3H), 7.28 (d, J=0.9 Hz, 1H). m/z (ES+APCI)⁺: 182/184 [M+H]⁺.

### Intermediate 10

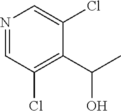

Methyl magnesium chloride, 3M in THF (11.5 ml, 34.4 mmol) in THF (10 ml) was added dropwise to a stirred suspension of 3,5-dichloropyridine-4-carboxaldehyde (5.51 g, 31.3 mmol) in THF (110 ml) at −78° C. The mixture was stirred at −78° C. for 1 hour and then allowed to warm to rt and stirred at that temperature for a further 1 hour. The mixture was quenched with sat. NH₄Cl(aq) whilst ice-cooling was applied. The mixture was extracted with ethyl acetate and the organic phase was washed with brine, dried and concentrated. The residue was purified by flash column chromatography on silica gel eluting with 5:1 to 3:1 petrol-ethyl aceate to afford an off-white solid (3.65 g, 61%). ¹H NMR (400 MHz, DMSO-d₆) δ ppm 1.41 (d, J=6.4 Hz, 3H), 5.31-5.37 (m, 1H), 5.59 (d, J=4.1 Hz, 1H), 8.51 (s, 2H). m/z (ES+APCI)⁺: 192/194 [M+H]⁺.

### Intermediate 11

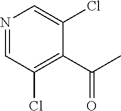

Freshly activated 4 Å molecular sieves (7.08 g) and NMO (5.53 g, 47.3 mmol) were added to a stirred solution of Intermediate 10 (3.63 g, 18.9 mmol) in DCM (125 ml). After 15 minutes TPAP (332 mg, 0.945 mmol) was added and the mixture was stirred at it for 45 minutes. The reaction mixture was filtered through Celite and the filtrate was concentrated. The residue was purified by flash column chromatography on silica gel in 5:1 petrol-ethyl acetate to give a yellow oil (2.57 g, 72%). ¹H NMR (400 MHz, DMSO-d₆) δ ppm 2.59 (s, 3H), 8.76 (s, 2H).

### Intermediate 12

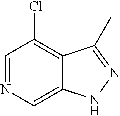

A mixture of Intermediate 11 (500 mg, 2.63 mmol) and 65% aqueous hydrazine (1.91 ml, 39.5 mmol) and n-butanol (10 ml) was irradiated in the 1-60 microwave reactor for 30 minutes at 200° C. The reaction was repeated 3 times on the same scale. The reaction mixtures were combined and diluted with water and ethyl acetate, and the organic phase was washed with brine, dried and concentrated. The residue was purified by flash column chromatography on silica gel eluting with 40:1 to 10:1 DCM-MeOH to give an off-white solid (1.05 g, 59%). ¹H NMR (400 MHz, DMSO-d₆) δ ppm 2.68 (s, 3H), 8.17 (s, 1H), 8.92 (s, 1H).

### Intermediate 13

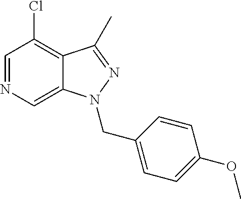 [SMILES: COc1ccc(Cn2nc(C)c3c(Cl)cncc32)cc1]

4-Methoxylbenzyl chloride (242 μl, 1.79 mmol) was added to a stirred mixture of Intermediate 12 (300 mg, 1.79 mmol) and potassium hydroxide (150 mg, 2.67 mmol) in DMF (20 ml), and the resulting mixture was stirred at rt overnight. The reaction mixture was quenched with water and extracted with ethyl acetate. The organic phase was washed with water (×3) and brine (×1), dried and concentrated. The residue was purified by flash column chromatography on silica gel in 2:1 to 1:2 petrol-ethyl acetate to afford the product (361 mg, 70%). ¹H NMR (400 MHz, DMSO-d₆) δ ppm 2.66 (s, 3H), 3.70 (s, 3H), 5.63 (s, 2H), 6.88 (m, 2H), 7.26 (m, 2H), 8.20 (s, 1H), 9.16 (s, 1H). m/z (ES+APCI)⁺: 288/290 [M+H]⁺.

### Intermediate 14

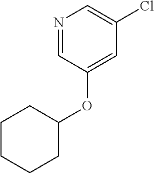 [SMILES: Clc1cncc(OC2CCCCC2)c1]

A solution of DIAD (11.3 ml, 57.7 mmol) in THF (40 ml) was added steadily to an ice-cooled solution of triphenylphosphine (15.1 g, 57.7 mmol), cyclohexanol (5.8 g, 57.7 mmol) and 3-chloro-5-hydroxypyridine (5.0 g, 38.5 mmol) in THF (160 ml). The reaction mixture was stirred at it for 48 hours. The mixture was concentrated to dryness and the residue was purified by flash column chromatography on silica gel eluting with 10:1 petrol-ethyl acetate to give a yellow oil (4.6 g, 56%). ¹H NMR (400 MHz, DMSO-d₆) δ ppm 1.20-1.31 (m, 1H), 1.32-1.57 (m, 5H), 1.63-1.76 (m, 2H), 1.85-1.97 (m, 2H), 4.46-4.54 (m, 1H), 7.62 (t, J=2.3 Hz, 1H), 8.17 (d, J=1.8 Hz, 1H), 8.24 (d, J=2.3 Hz, 1H). m/z (ES+APCI)⁺: 212/214 [M+H]⁺.

### Intermediate 15

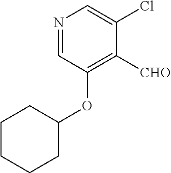 [SMILES: O=Cc1c(Cl)cncc1OC1CCCCC1]

n-Butyllithium, 1.6 M in hexanes (11.1 ml, 17.7 mmol) was added to THF (55 ml) at −78° C. A solution of Intermediate 14 (2.5 g, 11.8 mmol) in THF (10 ml) was added dropwise whilst maintaining the temperature below −74° C. and the mixture was then stirred at −78° C. for 1.5 hours. After this period a solution of ethyl formate (2.85 ml, 35.4 mmol) in THF (10 ml) was then added dropwise at −78° C. The reaction mixture was then stirred at this temperature for 3.5 hours before quenching with sat. NH₄Cl(aq). The mixture was allowed to warm to it and diluted with ethyl acetate and water. The organic phase was washed with brine, dried and concentrated. The residue was purified by flash column chromatography on silica gel in 5:1 petrol-ethyl acetate to give an orange oil (950 mg, 34%). ¹H NMR (400 MHz, DMSO-d₆) δ ppm 1.29-1.61 (m, 6H), 1.63-1.77 (m, 2H), 1.81-1.99 (m, 2H), 4.76 (dt, J=8.0, 4.2 Hz, 1H), 8.35 (s, 1H), 8.68 (s, 1H), 10.36 (s, 1H).

### Intermediate 16

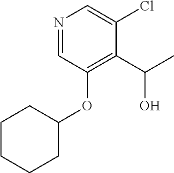 [SMILES: CC(O)c1c(Cl)cncc1OC1CCCCC1]

Methyl magnesium chloride, 3M in THF (1.45 ml, 4.35 mmol) in THF (5 ml) was added dropwise to a stirred solution of Intermediate 15 (950 mg, 3.96 mmol) in THF (70 ml) at −78° C. The reaction mixture was stirred for 45 minutes at −78° C. and then allowed to warm to rt where it was stirred for a further hour. The mixture was then cooled to −20° C. and quenched with sat. NH₄Cl(aq). The mixture was diluted with water and ethyl acetate. The organic phase was washed with brine, dried and concentrated, and the residue was purified by flash column chromatography on silica gel eluting with 3:1 petrol-ethyl acetate to give a yellow oil (667 mg, 66%). ¹H NMR (400 MHz, DMSO-d₆) δ ppm 1.28-1.59 (m, 9H), 1.62-1.76 (m, 2H), 1.83-1.96 (m, 2H), 4.58 (dt, J=8.0, 4.2 Hz, 1H), 5.05 (d, J=5.5 Hz, 1H), 5.27-5.35 (m, 1H), 8.14 (s, 1H), 8.32 (s, 1H).

### Intermediate 17

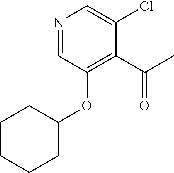 [SMILES: CC(=O)c1c(Cl)cncc1OC1CCCCC1]

Intermediate 16 (615 mg, 2.40 mmol), NMO (422 mg, 3.60 mmol) and 4 Å molecular sieves (800 mg) in DCM (30 ml) were stirred at rt for 15 minutes. TPAP (60 mg, 0.171 mmol) was then added and the reaction mixture was stirred at it for 3 hours. The mixture was filtered through Celite and the filtrate concentrated. The residue was purified by flash column chromatography on silica gel eluting with 3:1 petrol-ethyl acetate to give a yellow oil (488 mg, 80%). ¹H NMR (400 MHz, DMSO-d₆) δ ppm 1.24-1.53 (m, 6H), 1.56-1.71 (m, 2H), 1.84-1.94 (m, 2H), 2.48 (s, 3H), 4.66 (m 1H), 8.31 (s, 1H), 8.53 (s, 1H).

### Example 1

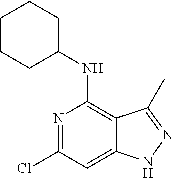 [SMILES: Cc1n[nH]c2cc(Cl)nc(NC3CCCCC3)c12]

A mixture of Intermediate 4 (33 mg, 0.163 mmol) and cyclohexylamine (19 μl, 0.163 mmol) in n-butanol (1 ml) was stirred at rt overnight, monitoring the reaction by LC/MS. The mixture was heated to 100° C. overnight and more cyclohexylamine (57 μl, 0.499 mmol) was added and stirring was continued at 100° C. overnight. The mixture was then diluted with ethyl acetate and washed with water and brine. The organic phase was dried and concentrated. The residue was purified by flash column chromatography on silica gel eluting with 2:1 petrol-ethyl acetate to give an off-white coloured solid (10 mg, 23%). ¹H NMR (400 MHz, DMSO-d₆) δ ppm 1.13-1.25 (m, 1H), 1.28-1.47 (m, 4H), 1.58-1.66 (m, 1H), 1.69-1.79 (m, 2H), 1.90-1.97 (m, 2H), 2.56 (s, 3H), 3.91-4.01 (m, 1H), 5.85 (d, J=7.8 Hz, 1H), 6.56 (s, 1H). m/z (ES+APCI)⁺: 265/267 [M+H]⁺

### Example 2

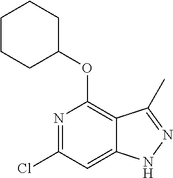 [SMILES: Cc1n[nH]c2cc(Cl)nc(OC3CCCCC3)c12]

Sodium hydride (60% dispersion in oil, 426 mg, 10.64 mmol) was added portionwise to a stirred solution of cyclohexanol (1.24 g, 12.38 mmol) in dioxane (15 ml) in a microwave reactor vial. The mixture was stirred at it for 45 minutes prior to addition of Intermediate 4 (500 mg, 2.48 mmol). The mixture was stirred at it overnight followed by irradiation in the Biotage 1-60 microwave reactor at 190° C. for 1 hour. The reaction mixture was added to ice and extracted with ethyl acetate. The organic phase was washed with brine, dried and concentrated. The residue was purified by flash column chromatography on silica gel eluting with 3:1 petrol-ethyl acetate to afford an oily solid, which was triturated with petrol to give a white solid (312 mg, 47%). ¹H NMR (400 MHz, DMSO-d₆) δ ppm 1.35-1.56 (m, 4H), 1.56-1.78 (m, 4H), 1.86-1.96 (m, 2H), 2.52 (s, 3H), 5.21 (dt, J=7.7, 3.7 Hz, 1H), 7.05 (s, 1H). m/z (ES+APCI)⁺: 266/268 [m+H]⁺.

### Example 3

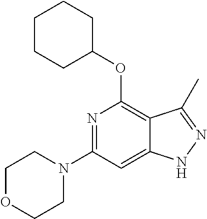 [SMILES: Cc1n[nH]c2cc(N3CCOCC3)nc(OC3CCCCC3)c12]

Example 2 (30 mg, 0.113 mmol) and morpholine (1 ml) were irradiated in the Biotage 1-60 microwave reactor at 200° C. for 5 hours. The mixture was concentrated to dryness and purified by preparative HPLC (high pH buffer), to give an off-white solid (5 mg, 14%). ¹H NMR (400 MHz, DMSO-d₆) δ ppm 1.37-1.53 (m, 4H), 1.57-1.77 (m, 4H), 1.85-1.93 (m, 2H), 2.43 (s, 3H), 3.25-3.33 (m, 4H), 3.68-3.74 (m, 4H), 5.17 (dt, J=7.4, 3.8 Hz, 1H), 5.99 (s, 1H). m/z (ES+APCI)⁺: 317 [M+H]⁺.

### Example 4

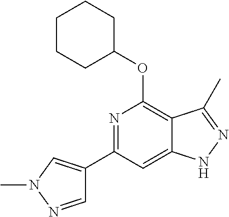 [SMILES: Cc1n[nH]c2cc(-c3cnn(C)c3)nc(OC3CCCCC3)c12]

A microwave reactor tube was charged with Example 2 (40 mg, 0.150 mmol), 1-methylpyrazole-4-boronic acid pinacol ester (47 mg, 0.226 mmol), Pd(dppf)Cl₂ (6.1 mg, 0.0075 mmol) and 2M Na₂CO₃(aq) (263 μl, 0.526 mmol) in dioxane (2 ml). The contents of the tube were degassed, placed under an atmosphere of nitrogen and irradiated in the Biotage 1-60 microwave reactor for 30 minutes at 160° C. The reaction mixture was diluted with ethyl acetate and water. The organic phase was washed with brine, dried and concentrated. Purification by preparative HPLC (high pH buffer) gave a pale brown solid (7 mg, 15%). ¹H NMR (400 MHz, DMSO-d₆) δ ppm 1.38-1.58 (m, 4H), 1.60-1.81 (m, 4H), 1.91-2.00 (m, 2H), 2.52 (s, 3H), 3.87 (s, 3H), 5.39 (dt, J=7.6, 4.0 Hz, 1H), 7.13 (s, 1H), 7.95 (s, 1H), 8.18 (s, 1H). m/z (ES+APCI)⁺: 312 [M+H]⁺

### Example 5

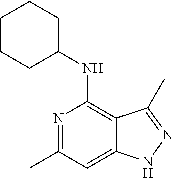 [SMILES: Cc1cc2[nH]nc(C)c2c(NC2CCCCC2)n1]

Intermediate 9 (50 mg, 0.275 mmol) and cyclohexylamine (63 μl, 0.549 mmol) in n-butanol (1 ml) were place in a sealed microwave reactor tube and irradiated in the 1-60 microwave reactor for 2 hours at 190° C. The mixture was concentrated to dryness and the residue was purified by preparative HPLC (high pH buffer), to give an off-white solid. Further purification by preparative HPLC (low pH buffer) gave a white solid (16 mg, 24%). ¹H NMR (400 MHz, DMSO-d₆) δ ppm 1.14-1.25 (m, 1H), 1.28-1.42 (m, 4H), 1.57-1.64 (m, 1H), 1.67-1.77 (m, 2H), 1.91-2.01 (m, 2H), 2.27 (s, 3H), 2.54 (s, 3H), 4.00-4.10 (m, 1H), 5.34 (d, J=7.8 Hz, 1H), 6.37 (s, 1H), 12.33 (s, 1H). m/z (ES+APCI)⁺: 245 [M+H]⁺.

### Example 6

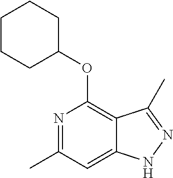 [SMILES: Cc1cc2[nH]nc(C)c2c(OC2CCCCC2)n1]

Sodium hydride, 60% dispersion in oil (38 mg, 0.961 mmol) was added to a stirred solution of cyclohexanol (116 μl, 1.10 mmol) in dioxane (3 ml) and the resulting mixture was stirred at it for 1 hour.

Intermediate 9 (50 mg, 0.275 mmol) was added and the reaction mixture was irradiated in the 1-60 microwave reactor for 1.5 hours at 180° C. The mixture was quenched with water and extracted with ethyl acetate. The organic phase was washed with brine, dried and concentrated. Purification by preparative HPLC (high pH buffer) gave a pale pink solid (18 mg, 27%). ¹H NMR (400 MHz, DMSO-d₆) 5 ppm 1.36-1.55 (m, 4H), 1.55-1.78 (m, 4H), 1.90 (m, 2H), 2.37 (d, J=0.9 Hz, 3H), 2.51 (s, 3H), 5.29 (m, 1H), 6.75 (d, J=0.9 Hz, 1H). m/z (ES+APC1)⁺: 246 [M+H]⁺.

### Example 7

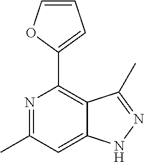 [SMILES: Cc1cc2[nH]nc(C)c2c(-c2ccco2)n1]

A mixture of Intermediate 9 (50 mg, 0.275 mmol), furan-2-boronic acid (46 mg, 0.412 mmol), Pd(dppf)Cl₂ (11 mg, 0.014 mmol) and 2M NaHCO₃(aq) (481 μl, 0.962 mmol) in dioxane (2 ml) were placed in a sealed microwave reactor tube, degassed and placed under an atmosphere of nitrogen. The mixture was irradiated in the 1-60 microwave reactor for 30 minutes at 160° C. The mixture was diluted with ethyl acetate and water, the organic phase was washed with brine, dried and concentrated. The residue was dissolved in DMSO (1 ml) and purified by preparative HPLC (high pH), to give a pale brown solid (30 mg, 51%). ¹H NMR (400 MHz, DMSO-d₆) δ ppm 2.54 (s, 3H), 2.62 (s, 3H), 6.69-6.72 (m, 1H), 7.11 (dd, J=3.2, 0.9 Hz, 1H), 7.18-7.21 (m, 1H), 7.94 (dd, J=1.8, 0.9 Hz, 1H). m/z (ES+APCI)⁺: 214 [M+H]⁺.

### Example 8

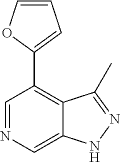

Intermediate 12 (40 mg, 0.238 mmol), furan-2-boronic acid (40 mg, 0.357 mmol), Pd(dppf)Cl₂ (10 mg, 0.012 mmol) and 2M NaHCO₃(aq) (417 μl, 0.833 mmol) and dioxane (2 ml) were placed in a sealed microwave reactor vial. The contents were degassed, placed under an atmosphere of nitrogen and irradiated in the 1-60 microwave reactor for 20 minutes at 160° C. The mixture was diluted with ethyl acetate and water. The organic phase was washed with brine, dried and concentrated. Purification by preparative HPLC (high pH buffer) afforded a pale brown solid (14 mg, 30%). ¹H NMR (400 MHz, DMSO-d₆) δ ppm 2.55 (s, 3H), 6.70-6.72 (m, 1H), 6.99-7.02 (m, 1H), 7.95 (dd, J=1.8, 0.9 Hz, 1H), 8.38 (s, 1H), 8.94 (s, 1H). m/z (ES+APCI)⁺: 200 [M+H]⁺.

### Example 9

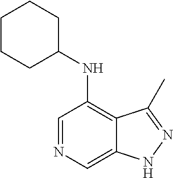 [SMILES: Cc1n[nH]c2cncc(NC3CCCCC3)c12]

### Step 1

A flask was charged with Intermediate 13 (150 mg, 0.521 mmol), xantphos (24 mg, 0.041 mmol), Pd₂(dba)₃ (28.5 mg, 0.031 mmol), cyclohexylamine (71 μl, 0.622 mmol) and sodium t-butoxide (150 mg, 1.56 mmol) in dioxane (6 ml). The resulting mixture was degassed and placed under an atmosphere of nitrogen and then heated at 100° C. overnight. On cooling to rt the mixture was diluted with water and ethyl acetate, and the organic phase was dried and concentrated. The residue was purified by flash column chromatography on silica gel in 3:1 ethyl acetate-petrol to provide a yellow solid (78 mg) which was impure and used in Step 2 without further purification.

### Step 2

The product from Step 1 (78 mg, 0.223 mmol) and aluminium (III) chloride (119 mg, 0.891 mmol) in toluene (7 ml) were heated at 50° C. for 4 hours. The reaction mixture was allowed to cool to it and concentrated to dryness. The residue was purified by flash column chromatography on silica gel in 20:1 DCM-MeOH to afford an orange oil. Further purification by preparative HPLC (high pH buffer) gave an off-white solid (6 mg, 12%). ¹H NMR (400 MHz, DMSO-d₆) δ ppm 1.16-1.45 (m, 5H), 1.58-1.66 (m, 1H), 1.67-1.77 (m, 2H), 1.98-2.08 (m, 2H), 2.60-2.68 (m, 3H), 3.35-3.46 (m, 1H), 4.85 (d, J=7.8 Hz, 1H), 7.41 (s, 1H), 8.13 (s, 1H). m/z (ES+APCI)⁺: 231 [M+H]⁺.

### Example 10

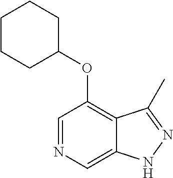 [SMILES: Cc1n[nH]c2cncc(OC3CCCCC3)c12]

A mixture of Intermediate 17 (100 mg, 0.394 mmol) and 65% aqueous hydrazine (282 μl, 0.582 mmol) in n-butanol (2.5 ml) was irradiated in the 1-60 microwave reactor for 1 hour at 200° C. The reaction mixture was concentrated to dryness. The residue was dissolved in DMSO (1 ml) and purified by preparative HPLC (high pH), to give a white solid (6 mg, 7%). ¹H NMR (400 MHz, DMSO-d₆) δ ppm 1.33-1.57 (m, 4H), 1.57-1.79 (m, 4H), 1.87-2.00 (m, 2H), 2.59 (s, 3H), 4.63-4.72 (m, 1H), 7.81 (s, 1H), 8.47 (s, 1H). m/z (ES+APCI)⁺: 232 [M+H]⁺.

### Intermediate 18

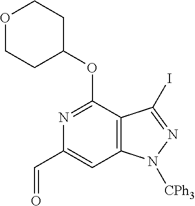

To a solution of 3-iodo-6-methyl-4-(tetrahydro-2H-pyran-4-yloxy)-1-trityl-1H-pyrazolo[4,3-c]pyridine (302 mg, 0.502 mmol) in 1,4-dioxane (8 mL) was added selenium dioxide (201 mg, 1.81 mmol). The reaction was stirred at 100° C. for 5 h. Additional selenium dioxide (200 mg, 1.8 mmol) was added. The reaction was further stirred at 100° C. for 18 h. The reaction was then filtered and concentrated. The crude product was purified by flash chromatography to give the desired aldehyde intermediate (0.2 g, 60%)

### Intermediate 19

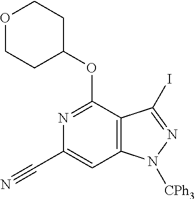

To a solution of 3-iodo-4-(tetrahydro-2H-pyran-4-yloxy)-1-trityl-1H-pyrazolo[4,3-c]pyridine-6-carbaldehyde (0.31 g, 0.50 mmol) in DMF (20 mL) was added hydroxylamine hydrochloride (38 mg, 0.55 mmol), triethylamine (0.078 mL, 0.55 mmol) and propanephosphonic acid cyclic trimer (0.400 g, 1.1 mmol). The reaction was stirred at 100° C. for 2 h. The reaction was diluted with sat. NaHCO₃ and extracted with EtOAc (3×). The combined extracts were washed with brine, dried over Na₂SO₄, filtered and concentrated. The crude product was purified by flash chromatography to give the desired product (0.31 g, quant.).

### Intermediate 20

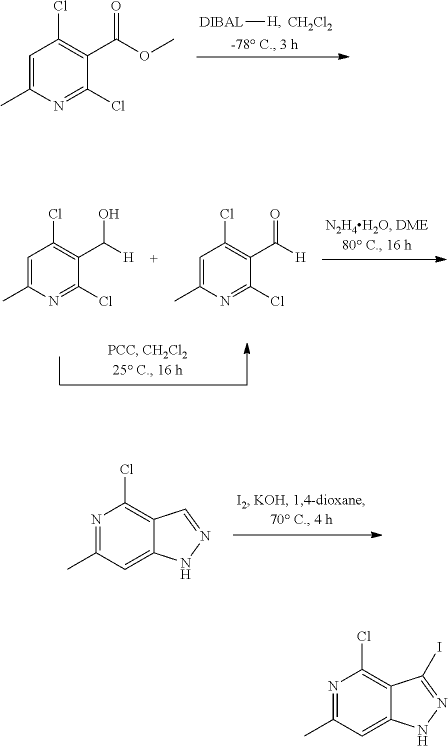

To a stirred solution of methyl 2,4-dichloro-6-methylnicotinate (45 g, 0.19 mol) in dry CH₂Cl₂ (450 mL) was added DIBAL-H (1 M solution in toluene) (576 mL, 0.57 mol) at −78° C. over a period of 30 minutes. The stirring was continued for another 2 h at −78° C. The reaction mixture was quenched with saturated NH₄Cl solution (100 mL) at the same temperature and allowed to warm to room temperature. The reaction mixture was diluted with 0.2 N HCl solution (1000 mL), extracted with CH₂Cl₂ (3×500 mL), the combined organics were washed with H₂O (2×200 mL), brine solution (2×250 mL), dried (Na₂SO₄) and concentrated. The crude compound was purified by flash column chromatography (silica gel, 100-200 mesh) and the desired aldehyde (XX) eluted with 5% EtOAc-pet ether to afford 16 g as an off-white solid. R_{f}: 0.6 (20% EtOAc/pet ether). (2,4-dichloro-6-methylpyridin-3-yl)methanol was eluted at 15% EtOAc-pet ether to afford 15 g as an off-white solid. R_{f}: 0.25 (20% EtOAc/pet ether), over all yield (31 g, 84%).

To a solution of (2,4-dichloro-6-methylpyridin-3-yl)methanol (15 g, 78.12 mmol) in CH₂Cl₂ (200 mL) was added PCC (42 g, 195.3 mmol) at 0° C. and stirred at room temperature for 16 h. The reaction mixture was concentrated and the residue was purified by flash column chromatography (silica gel, 100-200 mesh, eluted with 5% EtOAc-pet ether) to afford 2,4-dichloro-6-methylnicotinaldehyde (XX, 11.6 g, 78%) as off-white solid.

To a solution of compound 2,4-dichloro-6-methylnicotinaldehyde (18 g, 94.7 mmol) in 1,2-dimethoxy ethane (400 mL) was added 98% N₂H₄.H₂O (14.2 g, 284.2 mmol) at room temperature and heated at 80° C. for 16 h. The reaction mixture was concentrated and the residue was suspended in water (250 mL) and stirred for 30 minutes. The precipitated solid was collected by filtration and washed with pet. ether (2×250 mL). The obtained solid containing the minor 7-aza regioisomer was submitted for further purification. The mixture was suspended in CHCl₃ (150 mL), stirred for 30 minutes, filtered (repeated this process twice) and dried to obtain 4-chloro-6-methyl-1H-pyrazolo[4,3-c]pyridine (8 g, 50%) as a white solid.

To a stirred suspension of 4-chloro-6-methyl-1H-pyrazolo[4,3-c]pyridine (8 g, 47.9 mmol) and KOH (9.92 g, 177.2 mmol) in 1,4-dioxane (120 mL) was added I₂ (24.2 g, 95.8 mmol) at room temperature and heated at 70° C. for 4 h. The reaction mixture was cooled in an ice bath and added to a saturated sodium metabisulphite solution, stirred for 30 minutes, and the precipitated solid was collected by filtration and washed with water (600 mL), pet ether (2×200 mL) and dried to obtain 4-chloro-3-iodo-6-methyl-1H-pyrazolo[4,3-c]pyridine (11 g, 78%) as a white solid.

### Intermediate 21

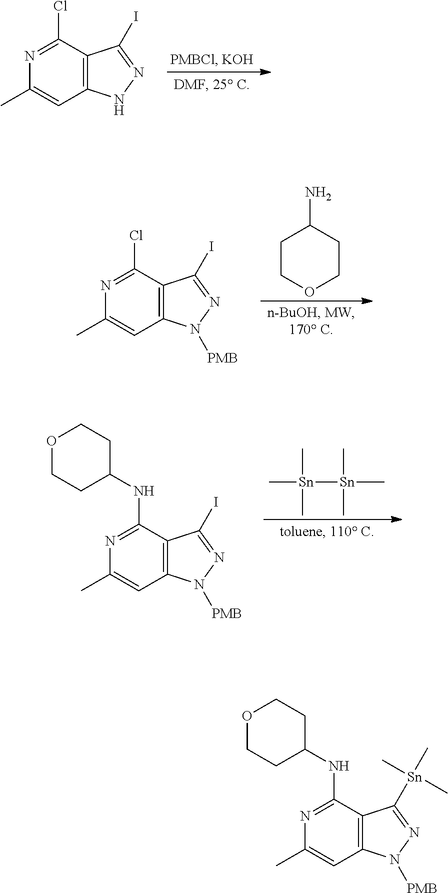

To a solution of 4-chloro-3-iodo-6-methyl-1H-pyrazolo[4,3-c]pyridine (3.00 g, 10.2 mmol) in DMF (30 mL) was added KOH (1.14 g, 20.4 mmol) and 1-(chloromethyl)-4-methoxybenzene (3.20 g, 20.4 mol). The mixture was stirred at room temperature overnight. The reaction mixture was then evaporated under reduced pressure and the residue was purified by flash column chromatography eluting with petroleum ether/ethyl acetate (from 10:1 to 8:1) to afford 4-chloro-3-iodo-1-(4-methoxybenzyl)-6-methyl-1H-pyrazolo[4,3-c]pyridine as a white solid (3.30 g, 62%).

A microwave vial equipped with a magnetic stirrer was charged with 4-chloro-3-iodo-1-(4-methoxybenzyl)-6-methyl-1H-pyrazolo[4,3-c]pyridine (2.60 g, 6.30 mmol), tetrahydro-2H-pyran-4-amine (1.91 g, 18.9 mmol), n-BuOH (10 mL), and diisopropylethylamine (2.44 g, 18.9 mmol). The reaction mixture was heated at 170° C. for 2 h under microwave irradiation. It was then cooled to room temperature and concentrated under reduced pressure. The resulting residue was purified by flash column chromatography eluting with DCM/petroleum ether/TEA (2:1:0.01) to afford 3-iodo-1-(4-methoxybenzyl)-6-methyl-N-(tetrahydro-2H-pyran-4-yl)-1H-pyrazolo[4,3-c]pyridin-4-amine as a white solid (2.35 g, 66%).

A sealed tube equipped with a magnetic stirrer was charged with 3-iodo-1-(4-methoxybenzyl)-6-methyl-N-(tetrahydro-2H-pyran-4-yl)-1H-pyrazolo[4,3-c]pyridin-4-amine (600 mg, 1.26 mmol), 1,1,1,2,2,2-hexamethyldistannane (618 mg, 1.88 mmol), toluene (12 mL), and trans-Pd(PPh₃)₂Cl₂ (24 mg, 0.0314 mol). After three cycles of vacuum/argon flash, the reaction mixture was heated at 110° C. for 15 h. It was then cooled to room temperature and filtered. The filtrate was concentrated under reduced pressure to afford the crude product (21, 860 mg), which was used without further purification in subsequent transformations.

### Intermediate 22

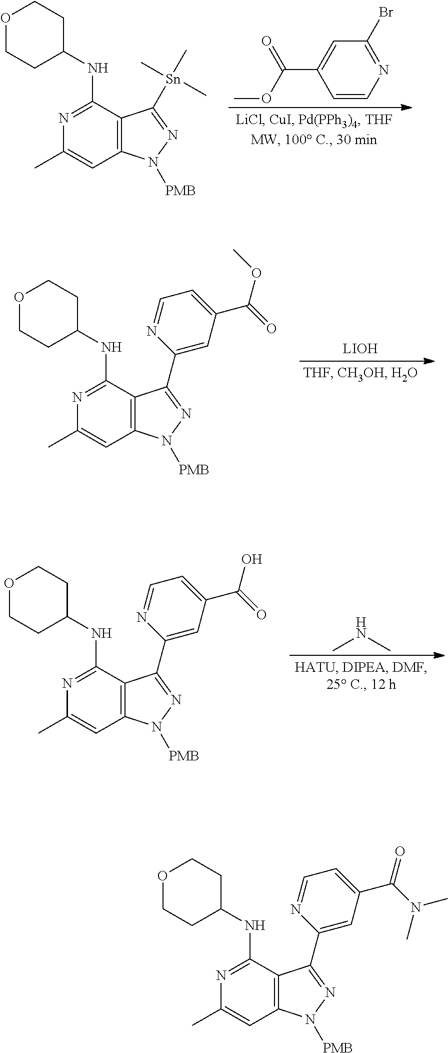

A microwave vial equipped with a magnetic stirrer was charged with crude 1-(4-methoxybenzyl)-6-methyl-N-(tetrahydro-2H-pyran-4-yl)-3-(trimethylstannyl)-1H-pyrazolo[4,3-c]pyridin-4-amine (550 mg, 1.07 mmol), methyl 2-bromoisonicotinate (276 mg, 1.28 mmol), LiCl (184 mg, 4.28 mmol), CuI (20 mg, 0.107 mmol), Pd(PPh₃)₄ (124 mg, 0.107 mmol), and THF (15 mL). After three cycles of vacuum/argon flash, the reaction mixture was heated at 100° C. for 1 h under microwave irradiation. It was then cooled to room temperature and filtered. The filtrate was concentrated under reduced pressure and the residue was purified by prep-TLC developing with petroleum ether/ethyl acetate (1:2) to afford methyl 2-(1-(4-methoxybenzyl)-6-methyl-4-(tetrahydro-2H-pyran-4-ylamino)-1H-pyrazolo[4,3-c]pyridin-3-yl)isonicotinate as a yellow solid (160 mg, 41%, 2 steps).

To a solution of THF (10 mL), methanol (10 mL) and H₂O (5 mL) was added methyl 2-(1-(4-methoxybenzyl)-6-methyl-4-(tetrahydro-2H-pyran-4-ylamino)-1H-pyrazolo[4,3-c]pyridin-3-yl)isonicotinate (160 mg, 0.329 mmol) and LiOH (131 mg, 3.29 mmol). The mixture was stirred at room temperature for 4 h and then concentrated at reduced pressure. H₂O (5 mL) and ethyl acetate (8 mL) were added to the residue and the resulting precipitate was collected to afford 2-(1-(4-methoxybenzyl)-4-(tetrahydro-2H-pyran-4-ylamino)-1H-pyrazolo[4,3-c]pyridine-3-yl)isonicotinic acid (140 mg, 90%) as a white solid.

To a 25-mL round bottom flask charged with 2-(1-(4-methoxybenzyl)-4-(tetrahydro-2H-pyran-4-ylamino)-1H-pyrazolo[4,3-c]pyridine-3-yl)isonicotinic acid (120 mg, 0.254 mmol) was added dimethylamine hydrochloride (107 mg, 1.27 mmol), HATU (193 mg, 0.508 mmol), diisopropylethylamine (0.5 mL), and DMF (3 mL). The reaction mixture was stirred at room temperature for 12 h. It was then purified by reverse-phase Combi-flash eluting with 0.3% NH₄HCO₃ in 1:3 water/CH₃CN to afford 2-(1-(4-methoxybenzyl)-6-methyl-4-(tetrahydro-2H-pyran-4-ylamino)-1H-pyrazolo[4,3-c]pyridin-3-yl)-N,N-dimethylisonicotinamide as a yellow solid (90 mg, 71%).

### Intermediate 23

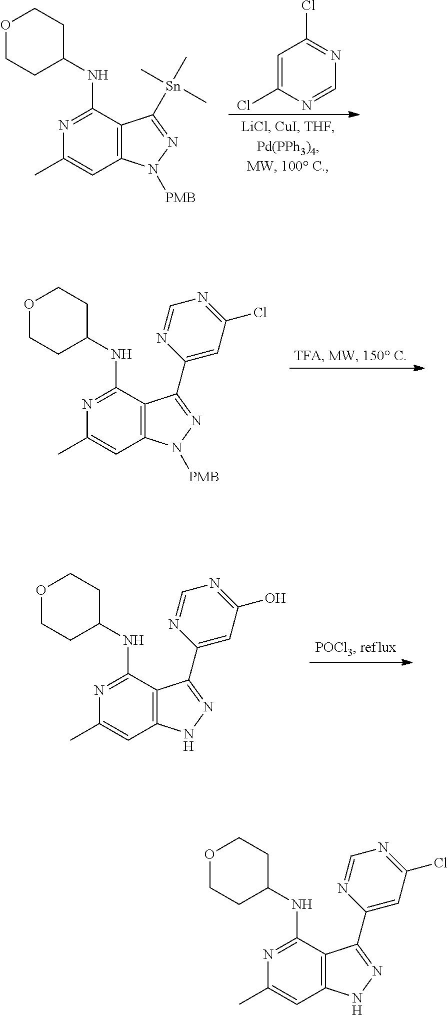

A microwave vial equipped with a magnetic stirrer was charged with 1-(4-methoxybenzyl)-6-methyl-N-(tetrahydro-2H-pyran-4-yl)-3-(trimethylstannyl)-1H-pyrazolo[4,3-c]pyridin-4-amine (600 mg, 1.16 mmol), 4,6-dichloropyrimidine (208 mg, 1.40 mmol), LiCl (195 mg, 4.64 mmol), CuI (22 mg, 0.116 mmol), Pd(PPh₃)₄ (134 mg, 0.116 mmol), and THF (10 mL). After three cycles of vacuum/argon flash, the reaction mixture was heated at 100° C. for 30 minutes under microwave irradiation. It was then cooled to room temperature and concentrated under reduced pressure. The resulting residue was purified by flash column chromatography eluting with petroleum ether/EA (2:1) to afford N-isopropyl-3-(pyridin-2-yl)-1-trityl-1H-pyrazolo[4,3-c]pyridin-4-amine as a yellow solid (130 mg, 24%).

A microwave vial equipped with a magnetic stirrer was charged with N-isopropyl-3-(pyridin-2-yl)-1-trityl-1H-pyrazolo[4,3-c]pyridin-4-amine (120 mg, 0.26 mmol) and TFA (15 mL). The reaction mixture was heated at 150° C. for 2 h under microwave irradiation. It was then cooled to room temperature and concentrated under reduced pressure. The pH of the resulting residue was adjusted to 7 by slowly introducing saturated NaHCO₃ solution. It was then extracted with ethyl acetate (2×50 mL). The combined organic phase was washed with water (50 mL) and brine, dried over anhydrous Na₂SO₄, filtered, and evaporated to afford 6-(6-methyl-4-(tetrahydro-2H-pyran-4-ylamino)-1H-pyrazolo[4,3-c]pyridin-3-yl)pyrimidin-4-ol (80 mg, 94%).

A mixture of 6-(6-methyl-4-(tetrahydro-2H-pyran-4-ylamino)-1H-pyrazolo[4,3-c]pyridin-3-yl) pyrimidin-4-ol (70 mg, 0.21 mmol) in POCl₃ (20 mL) was stirred at reflux for 5 h. It was then cooled to room temperature and concentrated under reduced pressure. The resulting residue was adjusted to a pH of 7 by slowly introducing saturated NaHCO₃ solution and extracted with ethyl acetate (2×50 mL). The combined organic phase was washed with water (50 mL) and brine, dried over anhydrous Na₂SO₄, filtered, and evaporated to afford the desired product as a white solid (50 mg, 69%).

### Intermediate 24

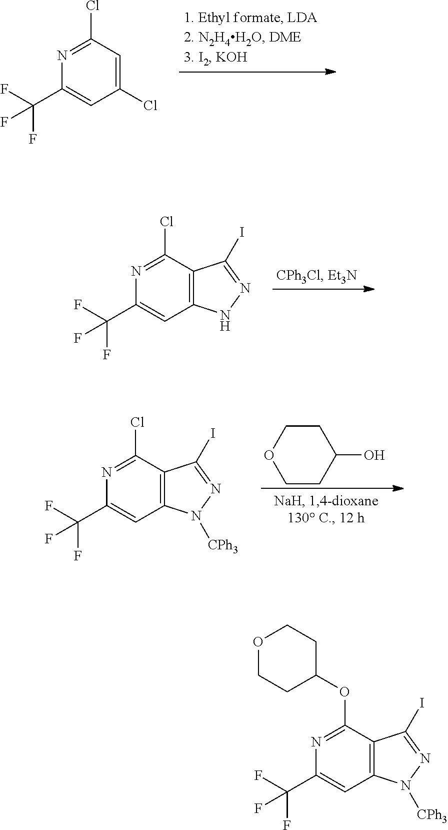

To a solution of diisopropylamine (1.6 g, 15.8 mmol) in dry THF (5 mL) at −78° C. was added n-BuLi (2.5 M in hexanes, 5.1 mL), and the resulting mixture was stirred at −10° C. for 1 h. To the above mixture was added a solution of 2,4-dichloro-6-(trifluoromethyl)pyridine (1.9 g, 8.84 mmol) in THF (2 mL), and the mixture was stirred at −78° C. for 40 minutes. A solution of freshly distilled ethylformate (0.37 g, 5 mmol) in THF (2 mL) was added over a period of 30 minutes to the above reaction mixture at −78° C. and stirred for 1 h. The reaction mixture was quenched with saturated aqueous NH₄Cl solution (10 mL), allowed to warm to room temperature and extracted with EtOAc (2×8 mL) and the combined organics were washed with water (2×5 mL), brine solution (2×5 mL), dried over Na₂SO₄ and concentrated to afford crude 2,4-dichloro-6-(trifluoromethyl)nicotinaldehyde (1.7 g, 81%) as a pale yellow solid. To the crude product dissolved in 1,2-dimethoxyethane (20 mL) was added 50% N₂H₄.H₂O (1.42 g, 13.95 mmol) at room temperature and then heated to 80° C. for 2 h. The reaction mixture was concentrated to afford the crude 4-chloro-6-(trifluoromethyl)-1H-pyrazolo[4,3-c]pyridine (200 mg, 13%) as a yellow solid. To a stirred suspension of the crude product and KOH (270 mg, 4.84 mmol) in 1,4-dioxane (40 mL) was added I₂ (663 mg, 2.61 mmol) at room temperature. The mixture was then heated at 70° C. for 3 h. The reaction mixture was then cooled in an ice bath and saturated aqueous sodium metabisulphite solution was added. The mixture was stirred for 30 minutes and the precipitate was collected by filtration. The filtrate was washed with water, dried (Na₂SO₄) and purified by flash column chromatography to give 4-chloro-3-iodo-6-(trifluoromethyl)-1H-pyrazolo[4,3-c]pyridine (70 mg, 3%, 3 steps) as a yellow solid.

To a solution of 4-chloro-3-iodo-6-(trifluoromethyl)-1H-pyrazolo[4,3-c]pyridine (0.06 g, 0.17 mmol) in methylene chloride (1.0 mL) was added triethylamine (0.036 mL, 0.26 mmol) and chlorotriphenylmethane (0.052 g, 0.18 mmol) and the reaction mixture stirred at room temperature for 12 h. The reaction was then concentrated and purified by flash chromatography to give 4-chloro-3-iodo-6-(trifluoromethyl)-1-trityl-1H-pyrazolo[4,3-c]pyridine (0.061 g, 60%).

To sodium hydride (4.96 mg, 0.12 mmol, 60%) suspended in 1,4-dioxane (0.5 mL) was added tetrahydro-4-pyranol (12.9 mg, 0.12 mmol) at room temperature in a microwave tube and the reaction mixture stirred for 30 min. 4-chloro-3-iodo-6-(trifluoromethyl)-1-trityl-1H-pyrazolo[4,3-c]pyridine (0.061 g, 0.1 mmol) in 1,4-dioxane (1.0 mL) was then added via cannula and the resulting mixture was heated to 130° C. for 12 h. The reaction was then diluted with methylene chloride, filtered, concentrated and purified by flash chromatography to give the desired product (0.012 g, 18%).

### Intermediate 25

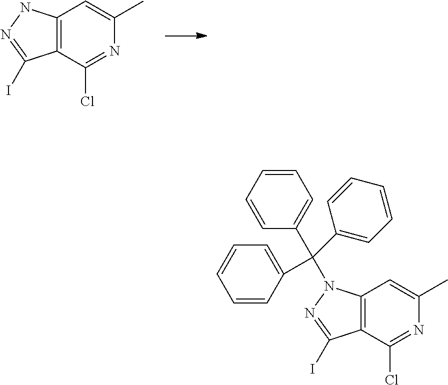 [SMILES: Cc1cc2[nH]nc(I)c2c(Cl)n1 ; Cc1cc2c(c(Cl)n1)c(I)nn2C(c1ccccc1)(c1ccccc1)c1ccccc1]

Triethylamine (499 uL, 0.00358 mol) was added to a suspension 4-chloro-3-iodo-6-methyl-1H-pyrazolo[4,3-c]pyridine (0.700 g, 0.00238 mol) and triphenylmethyl chloride (0.698 g, 0.00250 mol) in methylene chloride (19.7 mL, 0.308 mol) at room temperature. The reaction mixture became homogenous after ˜5 minutes. The reaction was stirred at room temperature for 3 h, diluted with water and extracted with methylene chloride. The organic layer was washed with brine, dried with Na₂SO₄, filtered, and concentrate to provide 4-chloro-3-iodo-6-methyl-1-trityl-1H-pyrazolo[4,3-c]pyridine as an off-white solid (1.24 g, 97%). ¹H-NMR (400 MHz, CDCl₃) δ 7.30 (m, 9H), 7.18-7.08 (m, 6H), 5.93 (s, 1H), 2.26 (s, 3H).

### Intermediate 26

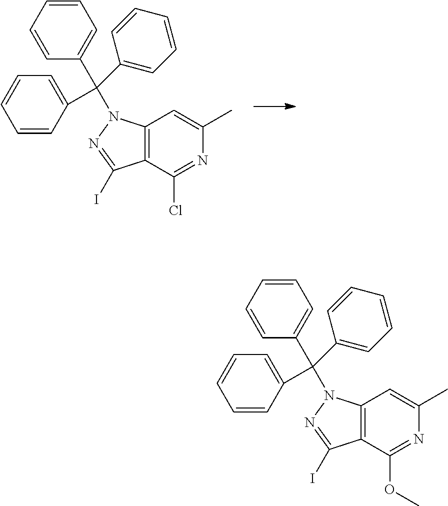 [SMILES: Cc1cc2c(c(Cl)n1)c(I)nn2C(c1ccccc1)(c1ccccc1)c1ccccc1 ; COc1nc(C)cc2c1c(I)nn2C(c1ccccc1)(c1ccccc1)c1ccccc1]

A 25% solution of sodium methoxide in methanol (25:75, sodium methoxide:methanol, 0.282 mL, 1.23 mmol) was added dropwise to a suspension of 4-chloro-3-iodo-6-methyl-1-trityl-1H-pyrazolo[4,3-c]pyridine (600 mg, 1 mmol) in tetrahydrofuran (5.4 mL, 67 mmol).

The resulting solution was heated for 2 h at 50° C. The volatile materials were removed by evaporation, and the resulting solid was partitioned between water and methylene chloride. The organic layer was dried (Na₂SO₄), filtered, and evaporated to provide 3-iodo-4-methoxy-6-methyl-1-trityl-1H-pyrazolo[4,3-c]pyridine (564 mg, 90%). ¹H-NMR (400 MHz, CDCl₃) δ 7.27 (dd, J=6.7, 2.3 Hz, 9H), 7.15 (dd, J=6.8, 2.9 Hz, 6H), 5.55 (s, 1H), 4.05 (s, 3H), 2.15 (s, 3H).

### Intermediate 27

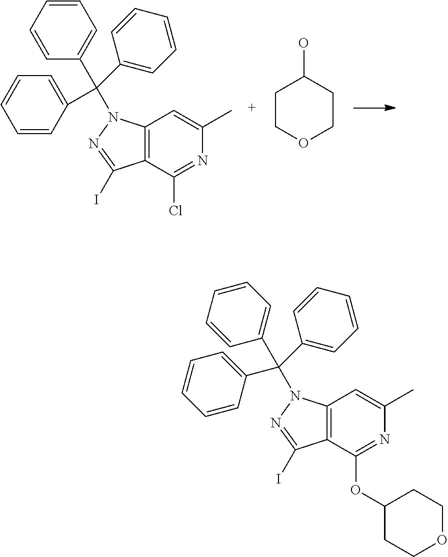

Tetrahydro-2H-pyran-4-ol (108 uL, 1.13 mmol) was added dropwise to a suspension of sodium hydride (61.0 mg, 1.52 mmol) in 1,4-dioxane (1 mL, 20 mmol). After bubbling had ceased, the suspension was stirred for 10 minutes, and then a solution of 4-chloro-3-iodo-6-methyl-1-trityl-1H-pyrazolo[4,3-c]pyridine (0.505 g, 0.942 mmol) in 1,4-dioxane (5 mL, 60 mmol) was added and the solution was heated to 180° C. for 1 h in the microwave. The reaction mixture was diluted with EtOAc and filtered through Celite. The solvent was removed, redissolved in 5 mL of EtOAc and left standing for 15 minutes. 10 mL of heptane were added, and the solution was stored at −10° C. for 5 h. The resulting solid was collected by filtration (˜250 mgs). The residue from evaporation of the filtrate was loaded onto silica, and the product was purified using flash column chromatography (24 g column, 0% to 50% EtOAc/Heptane) to provide an additional 180 mg. ¹H-NMR (400 MHz, CDCl₃) δ 7.39-7.21 (m, 10H), 7.16 (dd, J=6.8, 2.9 Hz, 5H), 5.63-5.54 (m, 1H), 5.53 (s, 1H), 4.13 (ddd, J=11.5, 8.3, 3.3 Hz, 2H), 3.83-3.63 (m, 2H), 2.12 (s, 3H), 2.09-2.00 (m, 2H), 1.97-1.82 (m, 2H).

### Intermediate 28

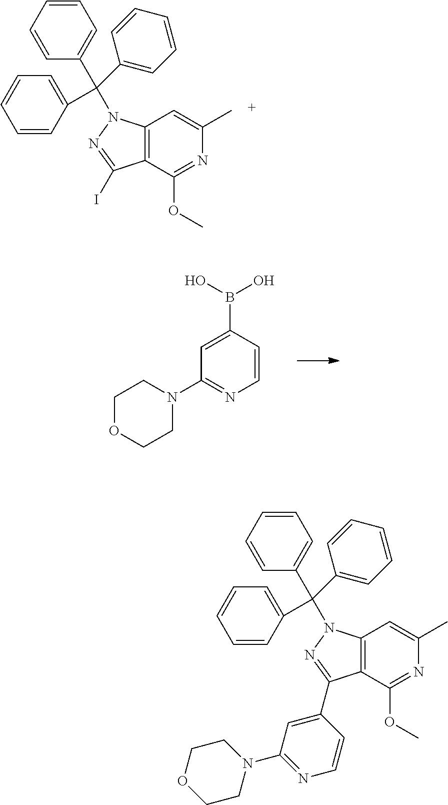 [SMILES: COc1nc(C)cc2c1c(I)nn2C(c1ccccc1)(c1ccccc1)c1ccccc1 ; OB(O)c1ccnc(N2CCOCC2)c1 ; COc1nc(C)cc2c1c(-c1ccnc(N3CCOCC3)c1)nn2C(c1ccccc1)(c1ccccc1)c1ccccc1]

3-Iodo-4-methoxy-6-methyl-1-trityl-1H-pyrazolo[4,3-c]pyridine (175 mg, 0.329 mmol), 2-morpholinopyridin-4-ylboronic acid (95.9 mg, 0.461 mmol), bis(di-tert-butyl(4-dimethylaminophenyl)phosphine)dichloropalladium(II) (23.3 mg, 0.0329 mmol), potassium acetate (45.2 mg, 0.461 mmol), and sodium carbonate (48.9 mg, 0.461 mmol) were loaded into a microwave vial that was purged with nitrogen. Acetonitrile (2.41 mL, 46.1 mmol) and water (0.593 mL, 32.9 mmol) were added, and the solution was purged with nitrogen for 10 minutes. The mixture was heated to 150° C. for 30 minutes. When the reaction was complete, the mixture was diluted with 15 mL of EtOAc, filtered through Celite and concentrated to give 4-(4-(4-methoxy-6-methyl-1-trityl-1H-pyrazolo[4,3-c]pyridin-3-yl)pyridin-2-yl)morpholine.

### Intermediate 29

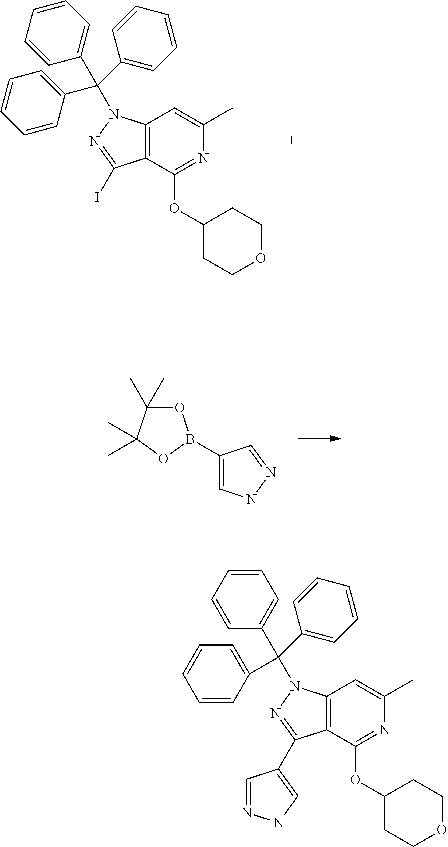 [SMILES: Cc1cc2c(c(I)nn2C(c2ccccc2)(c2ccccc2)c2ccccc2)c(OC2CCOCC2)n1 ; CC1(C)OB(c2cn[nH]c2)OC1(C)C ; Cc1cc2c(c(OC3CCOCC3)n1)c(-c1cn[nH]c1)nn2C(c1ccccc1)(c1ccccc1)c1ccccc1]

3-Iodo-6-methyl-4-(tetrahydro-2H-pyran-4-yloxy)-1-trityl-1H-pyrazolo[4,3-c]pyridine (500 mg, 0.8 mmol), 4,4,5,5-tetramethyl-2-(1H-pyrazol-4-yl)-1,3,2-dioxaborolane (226 mg, 1.16 mmol), bis(di-tert-butyl(4-dimethylaminophenyl)phosphine)dichloropalladium(II) (58.9 mg, 0.0831 mmol), potassium acetate (114 mg, 1.16 mmol), and Sodium carbonate (123 mg, 1.16 mmol) were loaded into a microwave vial that was purged with nitrogen. Acetonitrile (6.08 mL, 116 mmol) and water (1.50 mL, 83.1 mmol) were added, and the solution was and purged with nitrogen for 10 minutes. The reaction mixture was heated to 150° C. for 30 minutes in the microwave. The reaction mixture was filtered, washed with EtOAc, evaporated, and partitioned between CH₂Cl₂ and water. The organic layer was collected, dried over Na₂SO₄, then filtered and evaporated. The resulting 6-methyl-3-(1H-pyrazol-4-yl)-4-(tetrahydro-2H-pyran-4-yloxy)-1-trityl-1H-pyrazolo[4,3-c]pyridine (462 mg, 92%) was used directly in the subsequent reaction.

### Intermediate 30

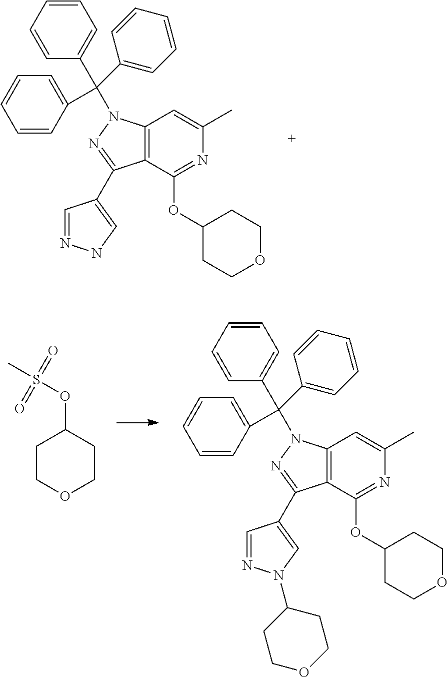

6-Methyl-3-(1H-pyrazol-4-yl)-4-(tetrahydro-2H-pyran-4-yloxy)-1-trityl-1H-pyrazolo[4,3-c]pyridine (464 mg, 0.857 mmol), tetrahydro-2H-pyran-4-yl methanesulfonate (290 mg, 1.6 mmol), cesium carbonate (399 mg, 1.22 mmol), and tetra-n-butylammonium iodide (60.4 mg, 0.163 mmol) were loaded into a vial equipped with a stirbar. N,N-Dimethylformamide (1.90 mL, 24.5 mmol) was added, and the mixture was heated at 90° C. After 2 h, the reaction was cooled to room temperature, diluted with EtOAc and filtered through Celite. The resulting 6-methyl-3-(1-(tetrahydro-2H-pyran-4-yl)-1H-pyrazol-4-yl)-4-(tetrahydro-2H-pyran-4-yloxy)-1-trityl-1H-pyrazolo[4,3-c]pyridine was used directly in the following reaction.

### Example 11

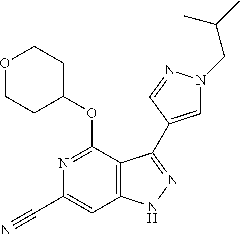 [SMILES: CC(C)Cn1cc(-c2n[nH]c3cc(C#N)nc(OC4CCOCC4)c23)cn1]

To a microwave tube was added 3-iodo-4-(tetrahydro-2H-pyran-4-yloxy)-1-trityl-1H-pyrazolo[4,3-c]pyridine-6-carbonitrile (0.112 g, 0.18 mmol), 1-iso-butyl-1H-pyrazole-4-boronic acid pinacol ester (0.069 g, 0.27 mmol), bis(di-tert-butyl-(4-dimethylaminophenyl)phosphine)dichloropalladium (13 mg 0.018 mmol), 2 M sodium carbonate (0.18 mL) and acetonitrile (2.3 mL). The reaction was sealed and heated in the microwave at 140° C. for 20 minutes. The reaction was diluted with water and extracted with EtOAc (3×). The combined extracts were washed with brine, dried over Na₂SO₄, filtered and concentrated.

The crude product was then dissolved in DCM (3.4 mL) and TFA (0.028 mL). Triethylsilane (0.058 mL) was then added. The reaction was then stirred at room temperature for 30 minutes. The reaction was filtered and concentrated. The crude product was purified by reverse phase HPLC to give 3-(1-isobutyl-1H-pyrazol-4-yl)-4-(tetrahydro-2H-pyran-4-yloxy)-1H-pyrazolo[4,3-c]pyridine-6-carbonitrile (26.5 mg, 39.5%). ¹H-NMR (400 MHz, DMSO-d₆) δ ppm 8.56 (s, 1H), 8.19 (s, 1H), 7.89 (s, 1H), 7.32 (s, 1H), 7.24 (d, J=4.3, 1H), 3.81 (m, 3H), 3.60 (m, 4H), 3.52 (m, 2H), 3.17 (m, 2H), 2.91 (d, J=4.3, 3H). m/z (ES+APCI)⁺: 367.2 [M+H]⁺.

### Example 12

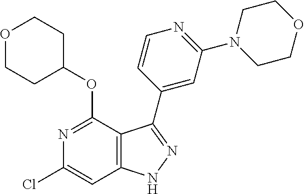 [SMILES: Clc1cc2[nH]nc(-c3ccnc(N4CCOCC4)c3)c2c(OC2CCOCC2)n1]

To a microwave tube was added 6-chloro-3-iodo-4-(tetrahydro-2H-pyran-4-yloxy)-1-trityl-1H-pyrazolo[4,3-c]pyridine (0.102 g, 0.16 mmol), 4-(4-(4,4,5,5-tetramethyl-1,3,2-dioxaborolan-2-yl)pyridin-2-yl)morpholine (0.057 g, 0.19 mmol), bis(di-tert-butyl-(4-dimethylaminophenyl)phosphine)dichloropalladium (11 mg 0.016 mmol), 2 M sodium carbonate (0.16 mL) and acetonitrile (2 mL). The reaction was sealed and heated in the microwave at 140° C. for 30 minutes. The reaction was diluted with water and extracted with EtOAc (3×). The combined extracts were washed with brine, dried over Na₂SO₄, filtered and concentrated.

The crude product was then dissolved in DCM (3 mL) and TFA (0.5 mL). Triethylsilane (0.1 mL) was then added. The reaction was then stirred at room temperature for 3 h. The reaction was filtered and concentrated. The crude product was purified by reverse phase HPLC to give 4-(4-(6-chloro-4-(tetrahydro-2H-pyran-4-yloxy)-1H-pyrazolo[4,3-c]pyridin-3-yl)pyridin-2-yl)morpholine (26.5 mg, 39.5%). ¹H-NMR (400 MHz, DMSO-d₅) δ ppm 8.23 (d, J=5.2, 1H), 7.26 (s, 2H), 7.21 (d, J=5.2, 1H), 5.42 (m, 1H), 3.73 (m, 4H), 3.50 (m, 4H), 2.05 (m, 2H), 1.67 (m, 2H). m/z (ES+APC0⁺: 416.1 [M+H]⁺.

### Example 13

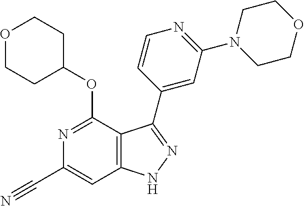 [SMILES: N#Cc1cc2[nH]nc(-c3ccnc(N4CCOCC4)c3)c2c(OC2CCOCC2)n1]

To a microwave tube was added 3-iodo-4-(tetrahydro-2H-pyran-4-yloxy)-1-trityl-1H-pyrazolo[4,3-c]pyridine-6-carbonitrile (0.099 g, 0.16 mmol), 4-(4-(4,4,5,5-tetramethyl-1,3,2-dioxaborolan-2-yl)pyridin-2-yl)morpholine (0.070 g, 0.24 mmol), bis(di-tert-butyl-(4-dimethylaminophenyl)phosphine)dichloropalladium (11 mg 0.016 mmol), 2 M sodium carbonate (0.16 mL) and acetonitrile (2 mL). The reaction was sealed and heated in the microwave at 140° C. for 30 minutes. The reaction was diluted with water and extracted with EtOAc (3×). The combined extracts were washed with brine, dried over Na₂SO₄, filtered and concentrated. The crude product was then dissolved in DCM (3 mL) and TFA (0.25 mL). Triethylsilane (0.05 mL) was then added. The reaction was then stirred at room temperature for 1 h. The reaction was filtered and concentrated. The crude product was purified by reverse phase HPLC to give the desired product (26.5 mg, 39.5%). ¹H-NMR (400 MHz, DMSO-d₆) δ ppm 8.24 (d, J=5.1, 1H), 8.00 (s, 1H), 7.26 (s, 1H), 7.20 (d, J=5.2, 1H), 5.47 (s, 1H), 3.72 (m, 6H), 3.52 (m, 6H), 2.05 (m, 2H), 1.69 (m, 2H). m/z (ES+APCI)⁺: 427.1 [M+H]⁺.

### Example 14

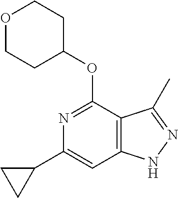 [SMILES: Cc1n[nH]c2cc(C3CC3)nc(OC3CCOCC3)c12]

To a microwave reaction vial was added 6-chloro-4-(cyclohexyloxy)-3-methyl-1H-pyrazolo[4,3-c]pyridine (49 mg, 0.18 mmol), cycloproylboronic acid (80 mg, 0.9 mmol), Pd(dppf)₂Cl₂ (15 mg, 0.018 mmol), 2 M Na₂CO₃ (0.28 mL, 0.6 mmol) and 1,4-dioxane (2 mL). The reaction was then sealed and heated in a microwave with stirring at 160° C. for 25 minutes. The reaction was then filtered and concentrated. The crude product was purified by reverse phase HPLC to give 4-(cyclohexyloxy)-6-cyclopropyl-3-methyl-1H-pyrazolo[4,3-c]pyridine (8.7 mg 17%). ¹H-NMR (400 MHz, DMSO-d₆) δ ppm 12.57 (s, 1H), 6.83 (s, 1H), 5.15 (m, 1H), 2.02 (m, 1H), 1.87 (m, 2H), 1.71 (m, 2H), 1.62 (m, 2H), 1.46 (m, 4H), 0.87 (m, 4H). m/z (ES+APCI)⁺: 272.1 [M+H]⁺.

### Example 15

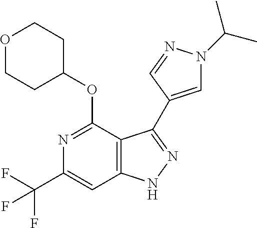 [SMILES: CC(C)n1cc(-c2n[nH]c3cc(C(F)(F)F)nc(OC4CCOCC4)c23)cn1]

To a microwave tube was added 3-(1-isopropyl-1H-pyrazol-4-yl)-4-(tetrahydro-2H-pyran-4-yloxy)-6-(trifluoromethyl)-1-trityl-1H-pyrazolo[4,3-c]pyridine (12 mg, 0.018 mmol), 1-isopropyl-4-(4,4,5,5-tetramethyl-1,3,2-dioxaborolan-2-yl)pyrazole (5.2 mg, 0.02 mmol), 1,1′-bis(diphenylphosphino)ferrocenepalladium (II) chloride (1.5 mg 0.002 mmol), 1 M potassium acetate (0.4 mL) and acetonitrile (1.6 mL). The reaction was degassed with nitrogen gas, sealed and heated in the microwave at 150° C. for 40 minutes. The reaction was diluted with water and extracted with EtOAc (3×). The combined extracts were washed with brine, dried over Na₂SO₄, filtered and concentrated. The crude product was dissolved in methylene chloride (0.8 mL), triethylsilane (0.012 mL, 0.07 mmol) and trifluoroacetic acid (0.7 mL, 9 mmol) at room temperature. The reaction was stirred for 15 minutes, concentrated with toluene (2 mL) and the crude product was purified by reverse phase HPLC to give 3-(1-isopropyl-1H-pyrazol-4-yl)-4-(tetrahydro-2H-pyran-4-yloxy)-6-(trifluoromethyl)-1H-pyrazolo[4,3-c]pyridine (3.1 mg, 43%). m/z (ES+APCI)⁺: 396.1 [M+H]⁺.

### Example 16

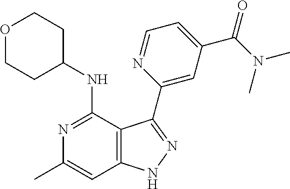 [SMILES: Cc1cc2[nH]nc(-c3cc(C(=O)N(C)C)ccn3)c2c(NC2CCOCC2)n1]

A microwave vial equipped with a magnetic stirrer was charged with 2-(1-(4-methoxybenzyl)-4-(tetrahydro-2H-pyran-4-ylamino)-1H-pyrazolo[4,3-c]pyridin-3-yl)-N,N-dimethylisonicotinamide (90 mg, 0.18 mmol) and TFA (3 mL). The reaction mixture was heated at 120° C. for 2 h under microwave irradiation. It was then cooled to room temperature and concentrated under reduced pressure. The resulting residue was purified by reverse-phase prep-HPLC to afford N,N-dimethyl-2-(6-methyl-4-(tetrahydro-2H-pyran-4-ylamino)-1H-pyrazolo[4,3-c]pyridin-3-yl)isonicotinamide as a yellow solid (25 mg, 37%). ¹H-NMR (500 MHz, MeOD) 58.74 (d, J=5.0, 1H), 8.36 (s, 1H), 7.43 (d, J=4.5, 1H), 6.49 (s, 1H), 4.33-4.35 (m, 1H), 4.03-4.07 (m, 2H), 3.65-3.69 (m, 2H), 3.17 (s, 3H), 3.06 (s, 3H), 2.41 (s, 3H), 2.18-2.20 (m, 2H), 1.70-1.73 (m, 2H). m/z (ES+APCI)⁺: 381 [M+H]⁺.

### Example 17

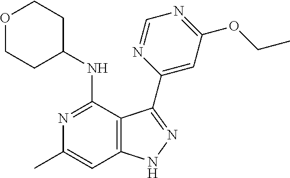 [SMILES: CCOc1cc(-c2n[nH]c3cc(C)nc(NC4CCOCC4)c23)ncn1]

A mixture of 3-(6-chloropyrimidin-4-yl)-6-methyl-N-(tetrahydro-2H-pyran-4-yl)-1H-pyrazolo[4,3-c]pyridin-4-amine (50 mg, 344 mmol) and a solution of 1 M NaOEt in EtOH (10 mL) and THF (5 mL) was stirred at reflux for 2 h. It was then cooled to room temperature and concentrated under reduced pressure. Then the mixture was extracted with ethyl acetate (2×50 mL). The combined organic phase was washed with water (50 mL) and brine, dried over anhydrous Na₂SO₄, and concentrated. The resulting residue was purified by reverse-phase prep-HPLC to afford 3-(6-ethoxypyrimidin-4-yl)-6-methyl-N-(tetrahydro-2H-pyran-4-yl)-1H-pyrazolo[4,3-c]pyridin-4-amine as a yellow solid (7 mg, 12%). ¹H-NMR (500 MHz, CDCl₃) δ 10.17 (s, 1H), 8.74 (s, 1H), 7.61 (s, 1H), 6.40 (s, 1H), 4.47-4.51 (m, 2H), 4.41-4.42 (m, 1H), 4.03-4.07 (m, 2H), 3.64-3.69 (m, 2H), 2.43 (s, 3H), 2.19-2.21 (m, 2H), 1.67-1.74 (m, 2H), 1.45 (t, 3H). m/z (ES+APCI)⁺: 355 [M+H]⁺.

### Example 18

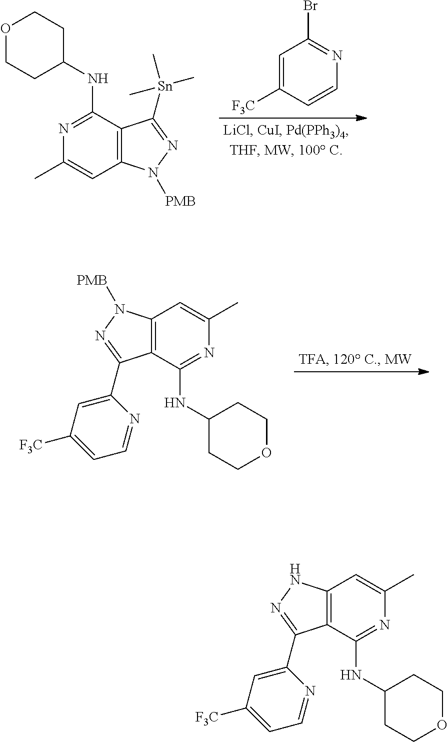

A microwave vial equipped with a magnetic stirrer was charged with 1-(4-methoxybenzyl)-6-methyl-N-(tetrahydro-2H-pyran-4-yl)-3-(trimethylstannyl)-1H-pyrazolo[4,3-c]pyridin-4-amine (190 mg, 0.369 mmol), 2-bromo-4-(trifluoromethyl)pyridine (167 mg, 0.738 mmol), LiCl (64 mg, 1.48 mmol), CuI (7 mg, 0.037 mmol), Pd(PPh₃)₄ (43 mg, 0.037 mmol), and THF (10 mL). After three cycles of vacuum/argon flash, the reaction mixture was heated at 100° C. for 1 h under microwave irradiation. It was then cooled to room temperature and filtered. The filtrate was concentrated under reduced pressure and the resulting residue was purified by reverse-phase Combi-flash eluting with 0.3% NH₄HCO₃ in 1:3 water/CH₃CN to afford 1-(4-methoxybenzyl)-6-methyl-N-(tetrahydro-2H-pyran-4-yl)-3-(4-(trifluoromethyl)pyridin-2-yl)-1H-pyrazolo[4,3-c]pyridin-4-amine as a yellow solid (55 mg). m/z (ES+APCI)⁺: 498 [M+H]⁺.

A microwave vial equipped with a magnetic stirrer was charged with 1-(4-methoxybenzyl)-6-methyl-N-(tetrahydro-2H-pyran-4-yl)-3-(4-(trifluoromethyl)pyridin-2-yl)-1H-pyrazolo[4,3-c]pyridin-4-amine (55 mg, 0.111 mmol) and TFA (3 mL). The reaction mixture was heated at 120° C. for 2 h under microwave irradiation. It was then cooled to room temperature and concentrated under reduced pressure. The resulting residue was purified by reverse-phase prep-HPLC to afford 6-methyl-N-(tetrahydro-2H-pyran-4-yl)-3-(4-(trifluoromethyl)pyridin-2-yl)-1H-pyrazolo[4,3-c]pyridin-4-amine as a yellow solid (21 mg, 50%). ¹H-NMR (500 MHz, CDCl₃) δ 9.96 (d, J=3.5, 1H), 8/3 (s, 1H), 8.61 (s, 1H), 0.51 (d, J=4.5, 1H), 6.43 (s, 1H), 4.41-4.42 (m, 1H), 4.03-4.07 (m, 2H), 3.67-3.69 (m, 2H), 2.49 (s, 3H), 2.18-2.30 (m, 2H), 1.65-1.73 (m, 2H). m/z (ES+APCI)⁺: 378 [M+H]⁺.

### Example 19

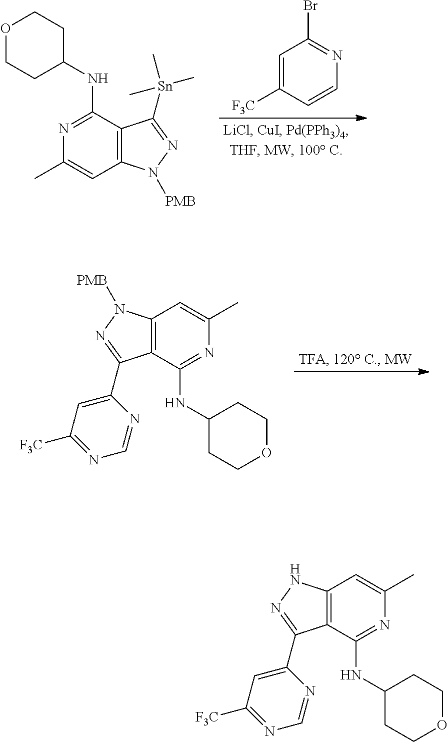

Following the procedures as described for compound Example 19 by substituting 2-bromo-4-(trifluoromethyl)pyridine with 4-bromo-6-(trifluoromethyl)pyrimidine, 6-methyl-N-(tetrahydro-2H-pyran-4-yl)-3-(6-(trifluoromethyl)pyrimidin-4-yl)-1H-pyrazolo[4,3-c]pyridin-4-amine was obtained as a yellow solid. ¹H-NMR (500 MHz, CDCl₃) δ 10.20 (s, 1H), 9.54 (s, 1H), 7.31 (s, 1H), 8.64 (s, 1H), 6.46 (s, 1H), 4.40-4.41 (m, 1H), 4.04-4.08 (m, 2H), 3.67 (t, 2H), 2.44 (s, 1H), 2.20-2.23 (m, 2H), 1.67-1.76 (m, 2H). m/z (ES+APCI)⁺: 379 [M+H]⁺.

### Example 20

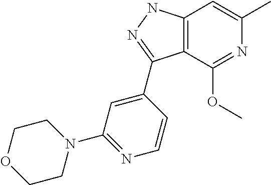 [SMILES: COc1nc(C)cc2[nH]nc(-c3ccnc(N4CCOCC4)c3)c12]

Trifluoroacetic Acid (2.0 mL, 26 mmol) was added dropwise to a solution of 4-(4-(4-methoxy-6-methyl-1-trityl-1H-pyrazolo[4,3-c]pyridin-3-yl)pyridin-2-yl)morpholine (0.187 g, 0.329 mmol) and triethylsilane (158 uL, 0.987 mmol) in methylene chloride (4.0 mL, 62 mmol) at 23° C. The reaction became red and was stirred for 15 minutes. The volatile materials were evaporated and the residue was purified by reverse phase HPLC to give 4-(4-(4-methoxy-6-methyl-1H-pyrazolo[4,3-c]pyridin-3-yl)pyridin-2-yl)morpholine. ¹H-NMR (400 MHz, DMSO) δ 13.26 (s, 1H), 7.57 (s, 1H), 7.39 (d, J=7.6 Hz, 1H), 7.30 (t, J=7.9 Hz, 1H), 6.99 (d, J=7.9 Hz, 1H), 6.94 (s, 1H), 3.96 (s, 3H), 3.86-3.67 (m, 4H), 3.22-3.08 (m, 4H), 2.45 (s, 3H). Note: Proton NMR is correct for formic acid adduct. m/z (ES+APCI)⁺: 367 [M+H]⁺.

### Example 21

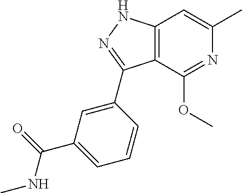

3-(4-Methoxy-6-methyl-1H-pyrazolo[4,3-c]pyridin-3-yl)-N-methylbenzamide was prepared according to a procedure as described in example 21. ¹H-NMR (400 MHz, DMSO) δ 13.41 (s, 1H), 8.45 (s, 1H), 8.08 (d, J=7.8 Hz, 1H), 7.84 (d, J=7.8 Hz, 1H), 7.54 (t, J=7.8 Hz, 1H), 6.97 (s, 1H), 3.96 (s, 3H), 2.81 (d, J=4.5 Hz, 3H), 2.46 (s, 3H). m/z (ES+APCI)⁺: 297 [M+H]⁺.

### Example 22

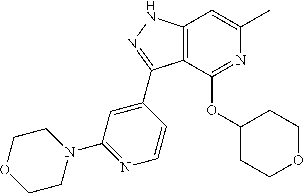 [SMILES: Cc1cc2[nH]nc(-c3ccnc(N4CCOCC4)c3)c2c(OC2CCOCC2)n1]

4-(4-(6-Methyl-4-(tetrahydro-2H-pyran-4-yloxy)-1H-pyrazolo[4,3-c]pyridin-3-yl)pyridin-2-yl)morpholine was prepared according to a procedure as described in example 21. ¹H-NMR (400 MHz, DMSO) δ 13.48 (s, 1H), 8.21 (d, J=5.1 Hz, 1H), 7.29 (s, 1H), 7.26 (d, J=5.2 Hz, 1H), 6.96 (s, 1H), 5.56-5.42 (m, 1H), 3.82-3.68 (m, 6H), 3.56-3.47 (m, 6H), 2.44 (s, 3H), 2.10-1.98 (m, 2H), 1.66 (dtd, J=12.5, 8.3, 3.9 Hz, 2H). m/z (ES+APCI)⁺: 397 [M+H]⁺.

### Example 23

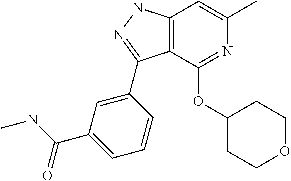 [SMILES: CNC(=O)c1cccc(-c2n[nH]c3cc(C)nc(OC4CCOCC4)c23)c1]

N-Methyl-3-(6-Methyl-4-(tetrahydro-2H-pyran-4-yloxy)-1H-pyrazolo[4,3-c]pyridin-3-yl)benzamide was prepared according to a procedure as described in example 21. ¹H-NMR (400 MHz, DMSO) δ 13.39 (s, 1H), 8.46 (d, J=4.4 Hz, 1H), 8.43 (d, J=1.5 Hz, 1H), 8.08 (d, J=7.7 Hz, 1H), 7.90-7.81 (m, 1H), 7.54 (t, J=7.7 Hz, 1H), 6.95 (s, 1H), 5.47 (tt, J=7.7, 3.8 Hz, 1H), 3.75-3.62 (m, 2H), 3.48 (ddd, J=11.4, 8.0, 3.2 Hz, 2H), 2.80 (d, J=4.5 Hz, 3H), 2.44 (s, 3H), 2.09-1.94 (m, 2H), 1.69 (dtd, J=12.0, 8.0, 3.8 Hz, 2H). m/z (ES+APCI)⁺: 367

### Example 24

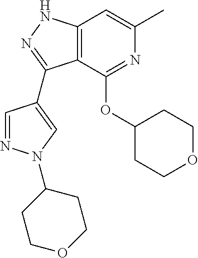 [SMILES: Cc1cc2[nH]nc(-c3cnn(C4CCOCC4)c3)c2c(OC2CCOCC2)n1]

6-Methyl-3-(1-(tetrahydro-2H-pyran-4-yl)-1H-pyrazol-4-yl)-4-(tetrahydro-2H-pyran-4-yloxy)-1H-pyrazolo[4,3-c]pyridine was prepared according to a procedure as described in example 21. ¹H-NMR (400 MHz, DMSO) δ 13.04 (s, 1H), 8.26 (s, 1H), 8.02 (s, 1H), 6.87 (s, 1H), 5.59-5.34 (m, 1H), 4.60-4.41 (m, 1H), 3.99 (d, J=11.4 Hz, 2H), 3.89 (dt, J=11.5, 4.1 Hz, 2H), 3.68-3.44 (m, 4H), 2.16 (d, J=9.5 Hz, 2H), 1.99 (ddd, J=19.5, 16.5, 9.8 Hz, 4H), 1.76 (qd, J=9.9, 4.2 Hz, 2H). m/z (ES+APCI)⁺: 384 [M+H]⁺.

### Example 25

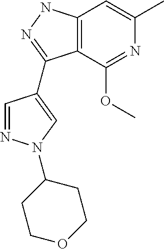 [SMILES: COc1nc(C)cc2[nH]nc(-c3cnn(C4CCOCC4)c3)c12]

4-Methoxy-6-methyl-3-(1-(tetrahydro-2H-pyran-4-yl)-1H-pyrazol-4-yl)-1H-pyrazolo[4,3-c]pyridine was prepared according to a procedure as described in example 21. ¹H-NMR 1H), 4.04 (s, 3H), 4.02-3.93 (m, 2H), 3.56-3.44 (m, 2H), 2.43 (s, 3H), 2.03 (dd, J=8.9, 3.7 Hz, 4H). m/z (ES+APCI)⁺: 314 [M+H]⁺.

Various modifications and variations of the described aspects of the invention will be apparent to those skilled in the art without departing from the scope and spirit of the invention. Although the invention has been described in connection with specific preferred embodiments, it should be understood that the invention as claimed should not be unduly limited to such specific embodiments. Indeed, various modifications of the described modes of carrying out the invention which are obvious to those skilled in the relevant fields are intended to be within the scope of the following claims.

### REFERENCES

1 Paisan-Ruiz, C., Jain, S., Evans, E. W., Gilks, W. P., Simon, J., van der Brug, M., Lopez de Munain, A., Aparicio, S., Gil, A. M., Khan, N., Johnson, J., Martinez, J. R., Nicholl, D., Carrera, I. M., Pena, A. S., de Silva, R., Lees, A., Marti-Masso, J. F., Perez-Tur, J., Wood, N. W. and Singleton, A. B. (2004) Cloning of the gene containing mutations that cause PARKS-linked Parkinson's disease. Neuron. 44, 595-600
2 Mata, I. F., Wedemeyer, W. J., Farrer, M. J., Taylor, J. P. and Gallo, K. A. (2006) LRRK2 in Parkinson's disease: protein domains and functional insights. Trends Neurosci. 29, 286-293
3 Taylor, J. P., Mata, I. F. and Fairer, M. J. (2006) LRRK2: a common pathway for parkinsonism, pathogenesis and prevention? Trends Mol. Med. 12, 76-82 Farrer, M., Stone, J., Mata, I. F., Lincoln, S., Kachergus, J., Hulihan, M., Strain, K. J. and Maraganore, D. M. (2005) LRRK2 mutations in Parkinson disease. Neurology. 65, 738-740
5 Zabetian, C. P., Samii, A., Mosley, A. D., Roberts, J. W., Leis, B. C., Yearout, D., Raskind, W. H. and Griffith, A. (2005) A clinic-based study of the LRRK2 gene in Parkinson disease yields new mutations. Neurology. 65, 741-744
6 Bosgraaf, L. and Van Haastert, P. J. (2003) Roc, a Ras/GTPase domain in complex proteins. Biochim Biophys Acta. 1643, 5-10
7 Marin, I. (2006) The Parkinson disease gene LRRK2: evolutionary and structural insights. Mol Biol Evol. 23, 2423-2433
8 Manning, G., Whyte, D. B., Martinez, R., Hunter, T. and Sudarsanam, S. (2002) The protein kinase complement of the human genome. Science. 298, 1912-1934
9 West, A. B., Moore, D. J., Biskup, S., Bugayenko, A., Smith, W. W., Ross, C. A., Dawson, V. L. and Dawson, T. M. (2005) Parkinson's disease-associated mutations in leucine-rich repeat kinase 2 augment kinase activity. Proc Natl Acad Sci USA. 102, 16842-16847
10 Greggio, E., Jain, S., Kingsbury, A., Bandopadhyay, R., Lewis, P., Kaganovich, A., van der Brug, M. P., Beilina, A., Blackinton, J., Thomas, K. J., Ahmad, R., Miller, D. W., Kesavapany, S., Singleton, A., Lees, A., Harvey, R. J., Harvey, K. and Cookson, M. R. (2006) Kinase activity is required for the toxic effects of mutant LRRK2/dardarin. Neurobiol Dis. 23, 329-341
11 Jaleel, M., Nichols, R. J., Deak, M., Campbell, D. G., Gillardon, F., Knebel, A. and Alessi, D. R. (2007) LRRK2 phosphorylates moesin at threonine-558: characterization of how Parkinson's disease mutants affect kinase activity. Biochem J. 405, 307-317
12 Goldberg, J. M., Bosgraaf, L., Van Haastert, P. J. and Smith, J. L. (2002)

Identification of four candidate cGMP targets in Dictyostelium. Proc Natl Acad Sci USA. 99, 6749-6754
13 Bosgraaf, L, Russcher, H., Smith, J. L., Wessels, D., Soli, D. R. and Van Haastert, P. J. (2002) A novel cGMP signalling pathway mediating myosin phosphorylation and chemotaxis in Dictyostelium. Embo J. 21, 4560-4570
14 Cohen, P. and Knebel, A. (2006) KESTREL: a powerful method for identifying the physiological substrates of protein kinases. Biochem J. 393, 1-6
15 Bretscher, A., Edwards, K. and Fehon, R. G. (2002) ERM proteins and merlin: integrators at the cell cortex. Nat Rev Mol Cell Biol. 3, 586-599
16 Polesello, C. and Payre, F. (2004) Small is beautiful: what flies tell us about ERM protein function in development. Trends Cell Biol. 14, 294-302
17 Nichols, R. J., Dzamko, N., Hutti, J. E., Cantley, L. C., Deak, M., Moran, J., Bamborough, P., Reith, A. D. and Alessi, D. R. (2009) Substrate specificity and inhibitors of LRRK2, a protein kinase mutated in Parkinson's disease. Biochem J. 424, 47-60

TABLE 1
Potency scores for selected compounds of the invention

| | |
|---|---|
| Example 1 | ** |
| Example 2 | ** |
| Example 3 | * |
| Example 4 | * |
| Example 5 | * |
| Example 6 | ** |
| Example 7 | ** |
| Example 8 | ** |
| Example 9 | ** |
| Example 10 | ** |

*** = LRRK2 IC50 <100 nM
** = LRRK2 IC50 between 100 nM and 1 μM
* = LRRK2 IC50 between 1 μM and 10 μM

TABLE 2
KI values for selected compounds of the invention

| | |
|---|---|
| Example 11 | *** |
| Example 12 | *** |
| Example 13 | ** |
| Example 14 | ** |
| Example 15 | *** |
| Example 16 | ** |
| Example 17 | *** |
| Example 18 | *** |
| Example 19 | *** |
| Example 20 | ** |
| Example 21 | ** |
| Example 22 | *** |
| Example 23 | ** |
| Example 24 | *** |
| Example 25 | *** |

*** = LRRK2 KI <100 nM
** = LRRK2 KI between 100 nM and 1 μM
* = LRRK2 KI between 1 μM and 10 μM

1. A compound of formula Ia or formula Ib, or a pharmaceutically acceptable salt or ester thereof, 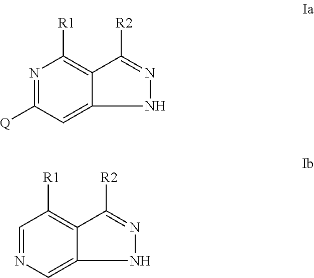
wherein:
R¹ is selected from:
aryl;
heteroaryl;
—NHR³;
fused aryl-C₄₋₇-heterocycloalkyl;
—CONR⁴R⁵;
—NHCOR⁶;
—C₃₋₇-cycloalkyl;
—NR³R⁶;
OR³;
OH;
NR⁴R⁵; and
—C₁₋₆ alkyl optionally substituted with a substituent selected from R¹¹ and a group A;
wherein said aryl, heteroaryl, fused aryl-C₄₋₇-heterocycloalkyl and C₄₋₇-heterocycloalkyl are each optionally substituted with one or more substituents selected from C₁₋₆-alkyl, C₃₋₇-cycloalkyl, heteroaryl, C₄₋₇-heterocycloalkyl, aryl and a group A, and said C₁₋₆-alkyl, C₃₋₇-cycloalkyl, heteroaryl, C₄₋₇-heterocycloalkyl, and aryl substituents are in turn each optionally substituted with one or more groups selected from R¹¹ and a group A;
R² is selected from hydrogen, aryl, C₁₋₆-alkyl, C₂₋₆-alkenyl, C₃₋₇-cycloalkyl, heteroaryl, C₄₋₇ heterocycloalkyl, fused aryl-C₄₋₇-heterocycloalkyl and halogen, wherein said C₁₋₆-alkyl, C₂₋₆-alkenyl, aryl, heteroaryl, fused aryl-C₄₋₇-heterocycloalkyl and C₄₋₇-heterocycloalkyl are each optionally substituted with one or more substituents selected from R¹¹ and A;
Q is a halogen, CN, or is selected from O₁₋₆-alkyl, C₃₋₇-cycloalkyl, heterocycloalkyl, aryl and heteroaryl, each of which is optionally substituted with one or more substituents A;
each R³ is selected from aryl, heteroaryl, C₄₋₇-heterocycloalkyl, C₃₋₇-cycloalkyl, fused aryl-C₄₋₇-heterocycloalkyl and C₁₋₆-alkyl, each of which is optionally substituted with one or more substituents selected from R¹¹ and A;
R⁴ and R⁵ are each independently selected from hydrogen, C₃₋₇-cycloalkyl, O₁₋₆-alkyl-C₃₋₇-cycloalkyl, aryl, heteroaryl, C₁₋₆-alkyl and a C₃₋₆-heterocycloalkyl ring optionally further containing one or more groups selected from oxygen, sulfur, nitrogen and CO, and optionally substituted by one or more R¹⁰ groups, wherein each C₁₋₆-alkyl, heteroaryl and aryl is optionally substituted by one or more substituents selected from C₁₋₆-alkyl, halogen, cyano, hydroxyl, aryl, halo-substituted aryl, heteroaryl, —NR⁸R⁹, —NR⁶R⁷, NR⁷(CO)R⁶, —NR⁷COOR⁶, —NR⁷(SO₂)R⁶, —COOR⁶, —CONR⁸R⁹, OR⁶, —SO₂R⁶ and a C₃₋₆-heterocycloalkyl ring optionally further containing one or more groups selected from oxygen, sulfur, nitrogen and CO and optionally substituted by one or more or R¹⁰ groups; or
R⁴ and R⁵ together with the N to which they are attached form a C₃₋₆-heterocycloalkyl ring optionally further containing one or more groups selected from oxygen, sulfur, nitrogen and CO, wherein said C₃₋₆-heterocycloalkyl ring is saturated or unsaturated and is optionally substituted with one or more groups selected from A, NR⁸R⁹ and R¹⁰;
each R⁶ is independently selected from C₁₋₆-alkyl, C₃₋₇ cycloalkyl, C₄₋₇-heterocycloalkyl, aryl and heteroaryl, each of which is optionally substituted by one or more substituents selected from R¹⁰, R¹¹ and A;
each R⁷ is selected from hydrogen, O₁₋₆-alkyl and C₃₋₇-cycloalkyl, wherein said C₁₋₆-alkyl is optionally substituted by one or more halogens;
each of R⁸ and R⁹ is independently selected from hydrogen and C₁₋₆-alkyl, wherein said C₁₋₆-alkyl group is optionally substituted by one or more halogens; or
R⁸ and R⁹ together with the N to which they are attached form a C₄₋₆-heterocycloalkyl ring optionally further containing one or more heteroatoms selected from oxygen and sulfur, wherein said C₄₋₆-heterocycloalkyl ring is optionally substituted by one or more R¹⁹ groups; and
each R¹⁰ is selected from C₃₋₇-cycloalkyl, aryl, heteroaryl, O-heteroaryl, aralkyl and C₁₋₆-alkyl, each of which is optionally substituted by one or more A groups, wherein where R¹⁰ is C₁₋₆-alkyl and two or more R¹⁰ groups are attached to the same carbon atom, the R¹⁰ groups may be linked to form a spiroalkyl group; and
each R¹¹ is independently selected from C₁₋₆-alkyl, C₃₋₇-cycloalkyl, C₁₋₆-alkyl-C₃₋₇-cycloalkyl, C₁₋₆-alkyl-heteroaryl, C₄₋₇-heterocycloalkyl, aryl and heteroaryl, each of which is optionally substituted with one or more substituents selected from A; and
A is selected from halogen, —NR⁴SO₂R⁵, —CN, —OR⁶, —NR⁴R⁵, —NR⁷R¹¹, hydroxyl, —CF₃, —CONR⁴R⁵, —NR⁴COR⁵, —NR⁷(CO)NR⁴R⁵, —NO₂, —CO₂H, —CO₂R⁶, —SO₂R⁶, —SO₂NR⁴R⁵, —NR⁴COR⁵, —NR⁴COOR⁵, C₁₋₆-alkyl, aryl and —COR⁶.

2. A compound according to claim 1 wherein R² is selected from:
hydrogen;
halogen, more preferably bromine;
aryl optionally substituted by one or more substituents selected from R¹¹ and A;
C₁₋₆-alkyl optionally substituted by one or more substituents selected from R¹¹ and A;
C₂₋₆-alkenyl optionally substituted by one or more A substituents;
C₃₋₇-cycloalkyl;
heteroaryl optionally substituted by one or more substituents selected from R¹¹ and A;
C₄₋₇-heterocycloalkyl; and
fused aryl-C₄₋₇-heterocycloalkyl.

3. A compound according to claim 1 wherein R² is selected from: aryl optionally substituted by one or more substituents selected from —NR⁴R⁵, —NR⁴COR⁵, —CONR⁴R⁵, OR⁶, halogen, optionally substituted C₁₋₆-alkyl, CN, C₄₋₇-heterocycloalkyl and heteroaryl;
C₁₋₆-alkyl optionally substituted by one or more substituents selected from —NR⁴COR⁵, —CONR⁴R⁵, —NR⁴R⁵, OR⁶, optionally substituted aryl, optionally substituted heteroaryl and C₄₋₇-heterocycloalkyl;
C₂₋₆-alkenyl optionally substituted by one or more —CONR⁴R⁵ substituents;
C₃₋₇-cycloalkyl, more preferably cyclopropyl;
heteroaryl optionally substituted by one or more substituents selected from —NR⁴R⁵, C₄₋₇-heterocycloalkyl, C₁₋₆-alkyl, C₃₋₇-cycloalkyl, C₁₋₆-alkyl-C₃₋₇-cycloalkyl and OR⁶;
C₄₋₇-heterocycloalkyl; and
fused aryl-C₄₋₇-heterocycloalkyl.

4. A compound according to claim 1 wherein R² is selected from:
a phenyl group optionally substituted by one or more substituents selected from —NHCO—C₁₋₆-alkyl, —CONHC₁₋₆-alkyl, CO—(N-morpholinyl), Cl, F, —OC₁₋₆-alkyl, —CONMe₂, OCF₃, CN, CF₃, C₁₋₆-alkyl-(A), N-morpholinyl and pyrazolyl;
a heteroaryl group selected from pyridinyl, quinolinyl, pyrazoyl, furanyl and pyrimidinyl, each of which may be optionally substituted by one or more substituents selected from C₁₋₆-alkyl, aralkyl, OC₁₋₆-alkyl, N-morpholinyl;
a C₁₋₆-alkyl group optionally substituted by one or more substituents selected from —CONR⁴R⁵, phenyl, pyridinyl and oxadiazolyl and piperidinyl, wherein said phenyl, pyridinyl and oxadiazolyl and piperidinyl groups are each optionally further substituted by one or more —NR⁴COR⁵, —CONR⁴R⁵, COR⁶, SO₂R⁶ or aryl groups.

5. A compound according to claim 1 wherein R² is selected from aryl, C₁₋₆-alkyl and heteroaryl, each of which is optionally substituted with one or more substituents selected from R¹¹ and A.

6. A compound according to claim 1 wherein R² is selected from aryl, C₁₋₆-alkyl and heteroaryl, each of which is optionally substituted with one or more substituents selected from CONR⁴R⁵, CF₃, C₁₋₆-alkyl, OR⁶ and C₄₋₇-heterocycloalkyl.

7. A compound according to claim 1 wherein R² is selected from C₁₋₆-alkyl, phenyl, pyridinyl, pyrimidinyl and pyrazolyl, each of which is optionally substituted by one or more substituents selected from CONMe₂, CF₃, iso-butyl, iso-propyl, OEt and morpholinyl.

8. A compound according to claim 1 wherein R² is selected from the following: Me

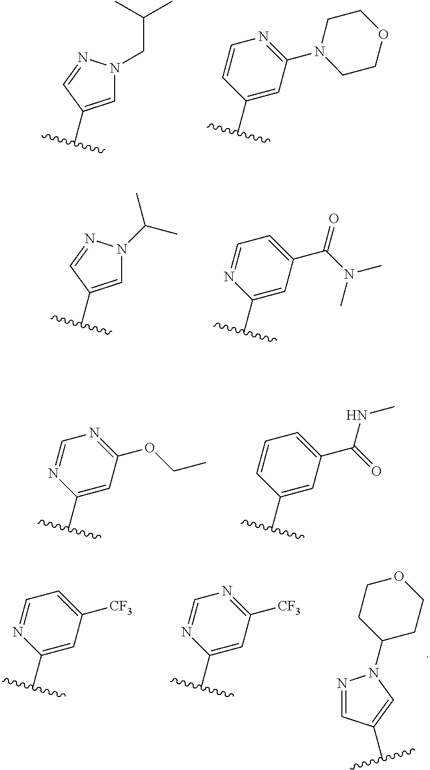

9. A compound according to claim 1 wherein R² is an unsubstituted C₁₋₆-alkyl group, more preferably methyl.

10. A compound according to claim 1 wherein R¹ is selected from:
—NHR³;
aryl;
heteroaryl;
C₄₋₇-heterocycloalkyl;
fused aryl-C₄₋₇-heterocycloalkyl;
—C₃₋₇-cycloalkyl;
—NR³R⁶;
OR³—
NR⁴R⁵; and
—C₁₋₆ alkyl optionally substituted with a substituent selected from R¹¹ and a group A;
wherein said aryl, heteroaryl, fused aryl-C₄₋₇-heterocycloalkyl and C₄₋₇-heterocycloalkyl are each optionally substituted with one or more substituents selected from C₁₋₆-alkyl, C₃₋₇-cycloalkyl, heteroaryl, C₄₋₇-heterocycloalkyl, aryl and a group A, and said C₁₋₆-alkyl, C₃₋₇-cycloalkyl, heteroaryl, C₄₋₇-heterocycloalkyl, and aryl substituents are in turn each optionally substituted with one or more groups selected from R¹¹ and a group A.

11. A compound according to claim 1 wherein R¹ is —NHR³, wherein R³ is selected from C₁₋₆-alkyl, C₃₋₇-cycloalkyl, C₄₋₇-heterocycloalkyl and aryl, each of which may be optionally substituted by one or more with one or more substituents selected from R¹¹ and A.

12. A compound according to claim 1 wherein R¹ is —NHR³ and R³ is selected from:
C₁₋₆-alkyl, optionally substituted by one or more —OR⁶, NR⁴COR⁵, heteroaryl, aryl, C₄₋₇-heterocycloalkyl, and C₃₋₇-cycloalkyl groups, wherein said aryl and heteroaryl groups are each independently optionally further substituted by one or more groups selected from CF₃, halogen, C₁₋₆-alkyl, —OR⁶ and —NR⁴R⁵;
a phenyl group optionally substituted by one or more substituents selected from —OR⁶, NR⁴COR⁵, —CONR⁴R⁵, aryl, —NR⁴R⁵, C₁₋₆-alkyl-heteroaryl, heteroaryl, halogen, —SO₂R⁶, CN, CF₃, C₁₋₆-alkyl, —SO₂NR⁴R⁵, —NR⁴SO₂R⁵, wherein said C₁₋₆-alkyl, heteroaryl and aryl groups are each independently optionally further substituted by one or more groups selected from CN, CF₃, halogen, C₁₋₆-alkyl, —OR⁶ and —NR⁴R⁵;
a heteroaryl group optionally substituted by one or more substituents selected from aryl, C₁₋₆-alkyl, and —NR⁴R⁵, wherein said aryl group is optionally further substituted by one or more A groups;
a C₄₋₇-heterocycloalkyl optionally substituted by one or more —COR⁶ groups;
a C₃₋₇-cycloalkyl group optionally substituted by one or more halogen or C₁₋₆-alkyl groups.

13. A compound according to claim 1 wherein R¹ is —OR³, wherein R³ is selected from C₁₋₆-alkyl, C₃₋₇-cycloalkyl, C₄₋₇-heterocycloalkyl and aryl, each of which may be optionally substituted by one or more with one or more substituents selected from R¹¹ and A.

14. A compound according to claim 13 wherein R¹ is —OR³, wherein R³ is C₁₋₆-alkyl, C₃₋₇-cycloalkyl or C₄₋₇-heterocycloalkyl, each of which may be optionally substituted by one or more A substituents.

15. A compound according to claim 1 wherein R¹ is selected from heteroaryl, —NHR³ and OR³, wherein said heteroaryl group is optionally substituted with one or more substituents seleted from the group A.

16. A compound according to claim 1 wherein R¹ is aryl or heteroaryl, each of which may be optionally substituted by one or more with one or more substituents selected from R¹¹ and A, more preferably R¹ is furyl.

17. A compound according to claim 1 wherein R¹ is —NH—C₃₋₇-cycloalkyl or NH—C₄₋₇-heterocycloalkyl, each of which may be optionally substituted by one or more A substituents.

18. A compound according to claim 1 wherein R³ is cyclohexyl or tetrahydropyranyl, each of which may be optionally substituted by one or more A substituents.

19. A compound according to claim 1 wherein R¹ is selected from the following:

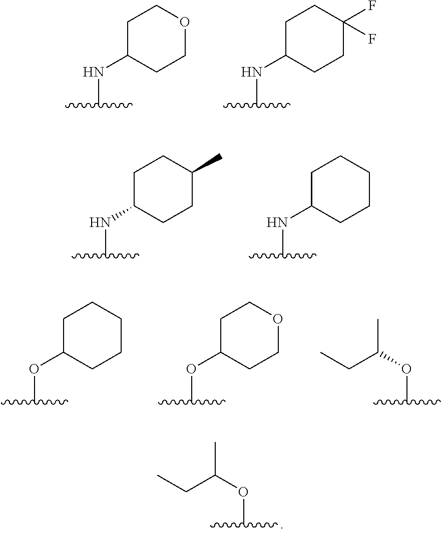

20. A compound according to claim 1 wherein R¹ is —OR³ or NHR³, and R³ is cyclohexyl, Me or tetrahydropyran-4-yl.

21. A compound according to claim 1 wherein Q is selected from a halogen, CN, O₁₋₆-alkyl, O₃₋₇-cycloalkyl, and C₄₋₇-heterocycloalkyl and heteroaryl, wherein said C₁₋₆-alkyl, C₃₋₇-cycloalkyl, C₄₋₇-heterocycloalkyl and heteroaryl are each independently optionally substituted with one or more substituents from the group A.

22. A compound according to claim 1 wherein Q is selected from CN, cyclopropyl, CF₃, chloro, methyl, N-morpholinyl and 1-methylpyrazol-4-yl.

23. A compound according to claim 1 which is selected from the following: 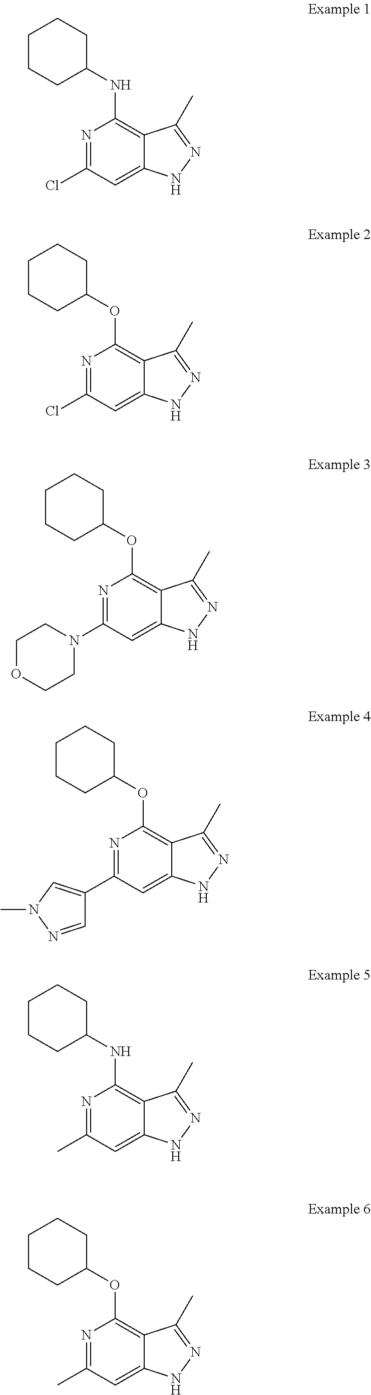 [SMILES: Cc1n[nH]c2cc(Cl)nc(NC3CCCCC3)c12 ; Cc1n[nH]c2cc(Cl)nc(OC3CCCCC3)c12 ; Cc1n[nH]c2cc(N3CCOCC3)nc(OC3CCCCC3)c12 ; Cc1n[nH]c2cc(-c3cnn(C)c3)nc(OC3CCCCC3)c12 ; Cc1cc2[nH]nc(C)c2c(NC2CCCCC2)n1 ; Cc1cc2[nH]nc(C)c2c(OC2CCCCC2)n1] 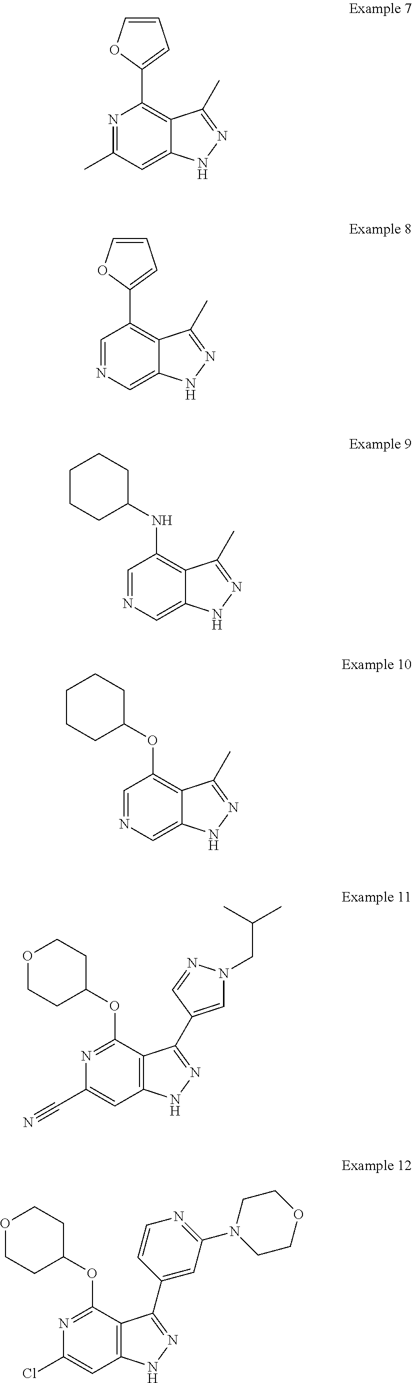 [SMILES: Cc1cc2[nH]nc(C)c2c(-c2ccco2)n1 ; Cc1n[nH]c2cncc(-c3ccco3)c12 ; Cc1n[nH]c2cncc(NC3CCCCC3)c12 ; Cc1n[nH]c2cncc(OC3CCCCC3)c12 ; CC(C)Cn1cc(-c2n[nH]c3cc(C#N)nc(OC4CCOCC4)c23)cn1 ; Clc1cc2[nH]nc(-c3ccnc(N4CCOCC4)c3)c2c(OC2CCOCC2)n1] 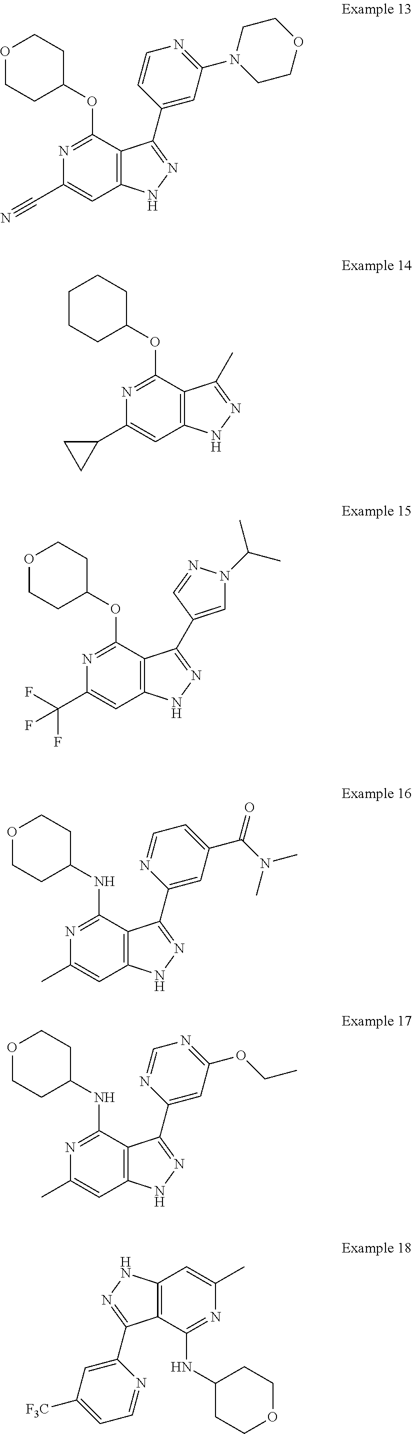 [SMILES: N#Cc1cc2[nH]nc(-c3ccnc(N4CCOCC4)c3)c2c(OC2CCOCC2)n1 ; Cc1n[nH]c2cc(C3CC3)nc(OC3CCCCC3)c12 ; CC(C)n1cc(-c2n[nH]c3cc(C(F)(F)F)nc(OC4CCOCC4)c23)cn1 ; Cc1cc2[nH]nc(-c3cc(C(=O)N(C)C)ccn3)c2c(NC2CCOCC2)n1 ; CCOc1cc(-c2n[nH]c3cc(C)nc(NC4CCOCC4)c23)ncn1 ; Cc1cc2[nH]nc(-c3cc(C(F)(F)F)ccn3)c2c(NC2CCOCC2)n1] 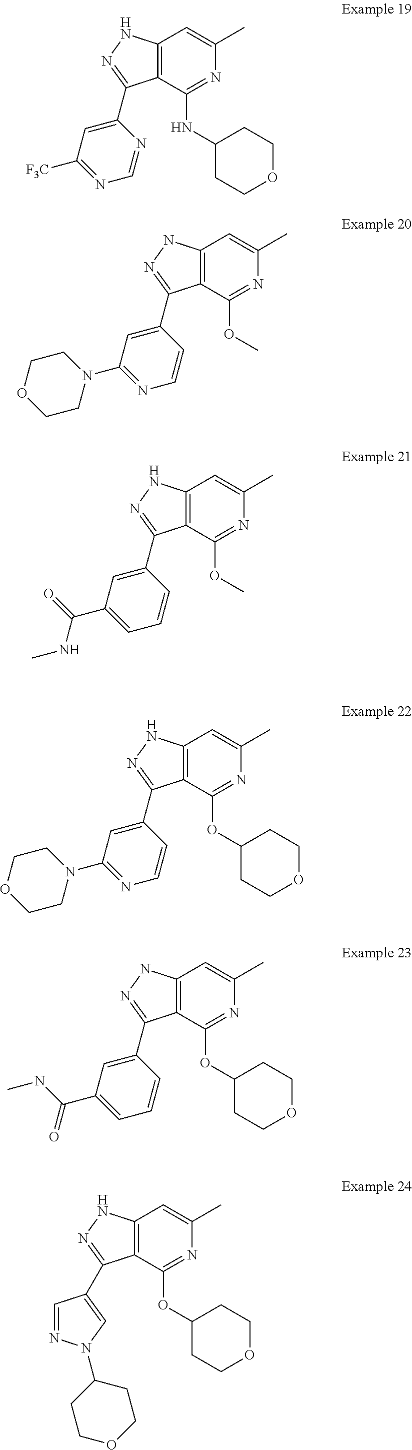 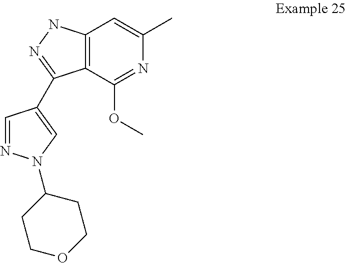 [SMILES: COc1nc(C)cc2[nH]nc(-c3cnn(C4CCOCC4)c3)c12]
or a pharmaceutically acceptable salt thereof.

24. A pharmaceutical composition comprising a compound according to claim 1 and a pharmaceutically acceptable carrier, diluent or excipient.

25. A compound according to claim 1 for use in medicine.

26. A method of treating cancer or neurodegenerative diseases in a subject, comprising administering to the subject a compound of claim 1.

27. (canceled)

28. A method of treating a disorder caused by, associated with or accompanied by abnormal kinase activity in a subject, comprising administering to the subject a compound of claim 1.

29. A method of treating a mammal having a disease state alleviated by the inhibition of LRRK2, wherein the method comprises administering to a mammal a therapeutically effective amount of a compound according to claim 1.

30. Use of a compound according to claim 1 in an assay for identifying further candidate compounds capable of inhibiting LRRK, more preferably LRRK2.

31. A process for preparing a compound of formula Ia′, wherein Q′ is halogen or C₁₋₆-alkyl and R¹ and R² are as defined in claim 1, said process comprising converting a compound of formula IIa′ into a compound of formula Ia′:

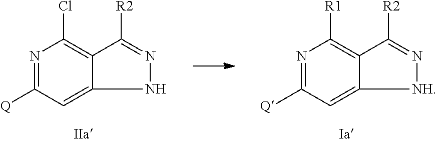

32. A process according to claim 31 which further comprises the step of preparing said compound of formula IIa′ by treating a compound of formula IIIa′ with hydrazine monohydrate:

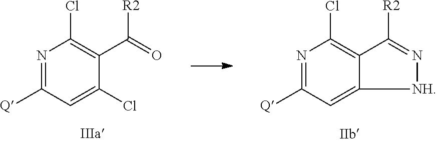

33. A process according to claim 32 which further comprises the step of preparing said compound of formula IIIa′ by treating a compound of formula IVa′ with an oxidizing agent:

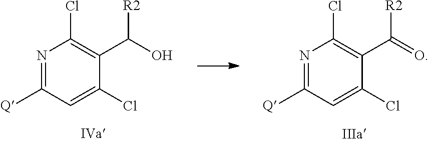

34. A process according to claim 33 which further comprises the step of preparing said compound of formula IVa′ by treating a compound of formula Va′ with R²—Mg—Cl:

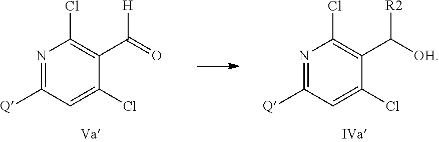

35. A process according to claim 1 where R¹ is —NHR³, and said process comprises reacting a compound of formula IIa′ with an amine of formula NH₂R³.

36. A process according to claim 1 where R¹ is an NH-containing C₄₋₇-heterocycloalkyl or an NH-containing fused aryl-C₄₋₇-heterocycloalkyl, and said process comprises reacting a compound of formula IIa′ with the NH-group of said C₄₋₇-heterocycloalkyl or fused aryl-C₄₋₇-heterocycloalkyl.

37. A process according to claim 1 wherein R¹ is selected from aryl, heteroaryl, C₄₋₇-heterocycloalkyl, fused aryl-C₄₋₇-heterocycloalkyl, —C₃₋₇ cycloalkyl and —C₁₋₆ alkyl, and said process comprises reacting a compound of formula IIa′ with X—R¹, where X is a 4,4,5,5-tetramethyl-1,3,2-dioxaborolan-2-yl group, in the presence of a coupling agent.

38. A process for preparing a compound of formula Ia″, wherein Q″ is C₃₋₇-cycloalkyl, heterocycloalkyl, aryl or heteroaryl, each of which is optionally substituted with one or more substituents A, and R¹ and R² are as defined in claim 1, said process comprising converting a compound of formula VIa″ into a compound of formula I:

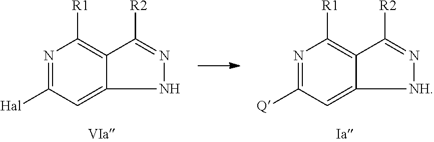

39. A process according to claim 38 which comprises reacting a compound of formula VIa″ with the NH-group of a C₄₋₇-heterocycloalkyl in the presence of a coupling agent.

40. A process according to claim 38 which comprises reacting a compound of formula VIa″ with a compound Q″-Y, where Q″ is C₃₋₇-cycloalkyl, heterocycloalkyl, aryl or heteroaryl and Y is a boronic acid or boronic acid ester moiety, in the presence of a coupling agent.

41. A process for preparing a compound of formula Ib as defined in claim 1, said process comprising converting a compound of formula IIb into a compound of formula Ib,

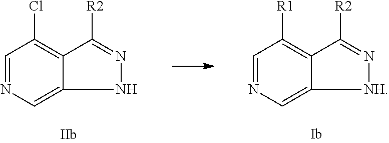

42. A process according to claim 41, where R¹ is aryl or heteroaryl, which comprises reacting said compound of formula IIb with a compound R¹—Y, where Y is a boronic acid or boronic acid ester moiety, in the presence of a coupling agent.

43. A process according to claim 41, where R¹ is —NHR³, which comprises reacting said compound of formula IIb with an amine of formula NH₂R³.

44. A process for preparing a compound of formula Ib as defined in claim 1, wherein R¹ is OR³, said process comprising converting a compound of formula IIIb into a compound of formula Ib,

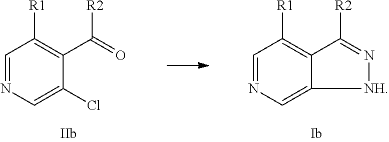

45. A combination comprising a compound according to claim 1 and a further therapeutic agent.

46. A pharmaceutical composition according to claim 24 which further comprises a second therapeutic agent.

47. The method of claim 28, wherein the abnormal kinase activity is abnormal LRRK2 activity.
